(19) United States
(12) Patent Application Publication
(10) Pub. No.: US 2015/0274664 A1
(43) Pub. Date: Oct. 1, 2015

(54) SUBSTITUTED CYCLOPROPYL COMPOUNDS, COMPOSITIONS CONTAINING SUCH COMPOUNDS AND METHODS OF TREATMENT

(71) Applicants: John J. ACTON, (US); Scott D. EDMONDSON, (US); Ping LIU, (US); Michael W. MILLER, (US); Harold B. WOOD, (US); Byron G. DUBOIS, (US); William B. GEISS, (US); MERCK SHARP & DOHME CORP., Rahway, NJ (US)

(72) Inventors: John J. Acton, Cranford, NJ (US); Scott D. Edmondson, Clark, NJ (US); Ping Liu, Westfield, NJ (US); Michael W. Miller, Scotch Plains, NJ (US); Harold B. Wood, Westfield, NJ (US); Byron G. DuBois, New York, NY (US); William B. Geiss, Athens, NY (US)

(73) Assignee: Marck Sharp & Dohme Corp., Rahway, NJ (US)

(21) Appl. No.: 14/431,366

(22) PCT Filed: Sep. 25, 2013
(86) PCT No.: PCT/US13/61567

§ 371 Date: Mar. 26, 2015

Related U.S. Application Data
(60) Provisional application No. 61/705,850, filed on Sep. 26, 2012.

(51) Int. Cl. C07D 211/60; C07D 401/12; C07D 413/04; C07D 401/14; C07D 401/04

(52) U.S. Cl. CPC C07D 211/60; C07D 401/14; C07D 401/04; C07D 413/04; C07D 401/12

(57) ABSTRACT

Substituted cyclopropyl piperidinyl compounds and pharmaceutically acceptable salts thereof are disclosed as useful for treating or preventing type 2 diabetes and similar conditions. The compounds are useful as agonists of the G-protein coupled receptor GPR-119. Pharmaceutical compositions and methods of treatment are also included.

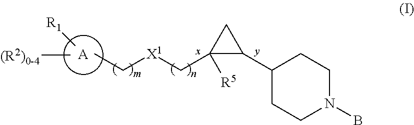

### BACKGROUND OF THE INVENTION

The present invention relates to G-protein coupled receptor agonists. In particular, the present invention is directed to agonists of GPR 119 that are useful for the treatment of diabetes, especially type 2 diabetes, as well as related diseases and conditions such as obesity and metabolic syndrome.

Diabetes is a disease derived from multiple causative factors. It is characterized by elevated levels of plasma glucose (hyperglycemia) in the fasting state or after administration of glucose during an oral glucose tolerance test. There are two generally recognized forms of diabetes. In type 1 diabetes, or insulin-dependent diabetes mellitus (IDDM), patients produce little or no insulin, the hormone which regulates glucose utilization. In type 2 diabetes, or noninsulin-dependent diabetes mellitus (T2DM), insulin is still produced in the body, and patients demonstrate resistance to the effects of insulin in stimulating glucose and lipid metabolism in the main insulin-sensitive tissues, namely, muscle, liver and adipose tissue. These patients often have normal levels of insulin, and may have hyperinsulinemia (elevated plasma insulin levels), as they compensate for the reduced effectiveness of insulin by secreting increased amounts of insulin.

There has been renewed focus on pancreatic islet-based insulin secretion that is controlled by glucose-dependent insulin secretion (GDIS). In this regard, several orphan G-protein coupled receptors (GPCR's) have recently been identified that are preferentially expressed in the β-cell and are implicated in GDIS. GPR119 is a cell-surface GPCR that is highly expressed in human (and rodent) islets as well as in insulin-secreting cell lines. Synthetic GPR119 agonists augment the release of insulin from isolated static mouse islets only under conditions of elevated glucose, and improve glucose tolerance in diabetic mice and diet-induced obese (DIO) C57/B6 mice without causing hypoglycemia. Novel GPR119 agonists therefore have the potential to function as anti-hyperglycemic agents that produce weight loss.

### SUMMARY OF THE INVENTION

The present invention addresses a compound represented by the formula:

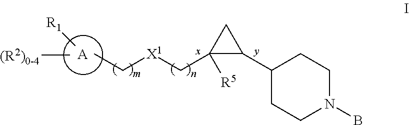

as well as the pharmaceutically acceptable salts thereof. The present invention further relates to methods of treating diabetes and related diseases and conditions.

### DETAILED DESCRIPTION OF THE INVENTION

The present invention addresses compounds represented by the formula:

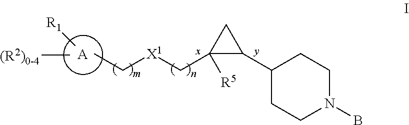

or a pharmaceutically acceptable salt thereof, wherein:
bonds x and y are in trans orientation in reference to one another;
ring A represents a 5- or 6-membered aryl or heteroaryl ring, the heteroaryl ring containing 1-4 heteroatoms, 1-4 of which are nitrogen atoms, and 0-1 of which are oxygen or sulfur atoms;
X¹ is oxygen or nitrogen;
m is an integer selected from 0-3;
n is an integer selected from 1-3; where m+n can be no more than 5;
R¹ is selected from:
   halogen;
   C₁₋₆alkyl;
   OC₁₋₆alkyl;
   C(O)C₁₋₆alkyl;
   C(O)C₃₋₆cycloalkyl;
   C(O)NHC₁₋₆alkyl;
   S(O)₀₋₂C₁₋₆alkyl;
   SO₂C₃₋₆cycloalkyl;
   SO₂NR^{b}R^{c}, wherein R^{b} and R^{c} are independently selected from H or C₁₋₆alkyl;
   —C₁₋₂alkylCONR^{d}R^{e} wherein R^{d} and R^{e} are independently selected from the group consisting of H, C₁₋₆alkyl, C₃₋₆cycloalkyl, haloC₁₋₆alkyl, haloC₃₋₆cycloalkyl, C(O)NH₂, C₁₋₆alkoxy, and C₃₋₆cycloalkylC₁₋₆alkoxy; wherein R^{d} and R^{e}, if individually alkyl, alkoxy or C(O)NH₂, can together form a 4-6 membered saturated heterocyclic ring having 1 nitrogen atom which 4-6 membered ring may be optionally substituted with 1-3 substituents independently selected from halogen, hydroxyl, oxo; C₁₋₆alkyl, C₁₋₆alkoxy, or CO₂C₁₋₆alkyl;
   —CH₂-heteroaryl, wherein heteroaryl is a 5-6 membered heteroaryl ring containing 1-4 heteroatoms selected from nitrogen or oxygen;
   or a 5-6 membered heteroaryl or saturated heterocyclic ring containing 1-4 heteroatoms, 1-4 of which are nitrogen atoms, and 0-1 of which are oxygen or sulfur atoms, wherein the R¹ alkyl, cycloalkyl, heteroaryl or heterocyclic moiety is optionally substituted with 1-3 of halogen; hydroxy; oxo; C₁₋₆alkyl; NH²; and O—C₁₋₆alkyl;

each R² is independently selected from halogen, CN, C₁₋₆alkyl and haloC₁₋₆alkyl;

R⁵ is hydrogen, C₁₋₃alkyl, C₁₋₃alkoxy, or cyano;
B represents (a) a 6 membered aryl ring or a 5-6 membered heteroaryl ring containing 1-4 heteroatoms, 1-4 of which are nitrogen atoms, and 0-1 of which are oxygen or sulfur atoms, said ring being optionally substituted with 1-3 groups selected from R³, wherein

each R³ is independently selected from:
halogen;
hydroxyl;
C₁₋₆alkyl;
C(O)OC₁₋₆alkyl;
C═O;
CN;
C₁₋₆alkoxy;
C₃₋₆cycloalkyl;
C(═O)—(O)ₙ—R′, wherein n is an integer from 0-3 and R′ is C₁₋₆alkyl or H;
5-6 membered heteroaryl ring containing 1-4 heteroatoms selected from nitrogen or oxygen;
C(═O)—R^{f}, wherein R^{f} is a 5-6 membered heteroaryl ring containing 1-4 heteroatoms independently selected from nitrogen or oxygen;
and haloC₁₋₆alkoxy;
   wherein the R³ alkyl moiety is optionally substituted with 1-3 substituents independently selected from:
halogen,
hydroxy,
C₁₋₆alkyl, or
C₁₋₆alkoxy;

Or b) CO₂R⁴, wherein

R⁴ is selected from:
C₁₋₆alkyl; or
C₃₋₆cycloalkyl,
wherein the alkyl and cycloalkyl are optionally substituted with 1-3 substituents independently selected from:
halogen,
C₁₋₆alkyl, or
C₃₋₆cycloalkyl.

Compounds in accordance herewith are equally potent to their cis counterparts.

The present invention is further directed to a compound of formula I-a:

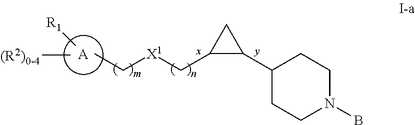

or a pharmaceutically acceptable salt thereof, wherein:
   bonds x and y are in trans orientation in reference to one another;
   ring A is a substituted phenyl, pyridyl or pyrimidine;
   X¹ is oxygen or nitrogen;
   m is an integer selected from 0-3;
   n is an integer selected from 1-3; where m+n can be no more than 5;
   B represents a) a pyrimidine ring optionally substituted with 1-2 groups selected from R³, wherein each R³ is independently selected from:
halogen;
hydroxyl;
C₁₋₆alkyl;
C(O)OC₁₋₆alkyl;
C═O;
CN;
C₁₋₆alkoxy;
C₃₋₆cycloalkyl;
C(═O)—(O)ₙ—R′, wherein n is an integer from 0-3 and R′ is C₁₋₆alkyl or H;
5-6 membered heteroaryl ring containing 1-4 heteroatoms selected from nitrogen or oxygen;
C(═O)—R^{f}, wherein R^{f} is a 5-6 membered heteroaryl ring containing 1-4 heteroatoms independently selected from nitrogen or oxygen;
or haloC₁₋₆alkoxy;
   wherein the R³ alkyl moiety is optionally substituted with 1-3 substituents independently selected from:
halogen,
hydroxy,
C₁₋₆alkyl, or
C₁₋₆alkoxy;
   b) 1,2,4-oxadiazol optionally substituted with 1-3 substituents independently selected from:
C₁₋₆alkyl,
C₁₋₆alkoxy; or
C₃₋₆cycloalkyl;

wherein the alkyl, alkoxy and cycloalkyl are optionally substituted with 1-3 substituents independently selected from:
Halogen;
C₁₋₆alkyl, or
C₃₋₆cycloalkyl;
   or c) CO₂R⁴, wherein

R⁴ is selected from:
C₁₋₆alkyl; or
C₃₋₆cycloalkyl,
wherein the alkyl and cycloalkyl are optionally substituted with 1-3 substituents independently selected from:
halogen,
C₁₋₆alkyl, or
C₃₋₆cycloalkyl;
   R¹ is selected from:
C₁₋₆alkyl;
OC₁₋₆alkyl;
C(O)C₁₋₆alkyl;
C(O)C₃₋₆cycloalkyl;
C(O)NHC₁₋₆alkyl;
S(O)₀₋₂C₁₋₆alkyl;
SO₂C₃₋₆cycloalkyl;
—C₁₋₂alkylCONR^{d}R^{e} wherein R^{d} and R^{e} are independently selected from H, C₁₋₆alkyl, C₃₋₆cycloalkyl, haloC₁₋₆alkyl, haloC₃₋₆cycloalkyl, C(O)NH₂, C₁₋₆alkoxy, or C₃₋₆cycloalkylC₁₋₆alkoxy; wherein R^{d} and R^{e}, if individually alkyl, alkoxy or C(O)NH₂, can together form a 4-6-membered saturated heterocyclic ring having 1 nitrogen atom which 4-6-membered ring may be optionally substituted with 1-3 substituents independently selected from halogen, hydroxy, oxo. C₁₋₆alkyl, C₁₋₆alkoxy; or CO₂C₁₋₆alkyl;
—CH₂-heteroaryl, wherein the heteroaryl is a 5-6 membered heteroaryl ring containing 1-4 heteroatoms independently selected from nitrogen or oxygen;
or a 5-6 membered heteroaryl or saturated heterocyclic ring containing 1-4 heteroatoms, 1-4 of which are nitrogen atoms, and 0-1 of which are oxygen or sulfur atoms,

wherein the R¹ alkyl, cycloalkyl, heteroaryl or heterocyclic moiety is optionally substituted with 1-3 substituents independently selected from: halogen; hydroxy; oxo; C₁₋₆alkyl; NH²; or O—C₁₋₆alkyl;

and R² is halogen which is further selected from fluoro or chloro.

The present invention is also directed to compounds of Formula I or I-a or pharmaceutically acceptable salts thereof wherein ring A is pyridyl. In distinct embodiments, the present invention is directed to compounds of Formula I or I-a or pharmaceutically acceptable salts thereof wherein ring A is phenyl or pyrimidine.

The present invention is also directed to compounds of Formula I or I-a or pharmaceutically acceptable salts thereof wherein m+n equals 5, 4, 3 or 2. In particular embodiments, m=1 and n=1. In distinct embodiments, m=0 and n=2.

The present invention is further directed to compounds of Formula I or I-a or pharmaceutically acceptable salts thereof, wherein B represents (b) CO₂R⁴, wherein R⁴ is
C₁₋₆alkyl, or
C₃₋₆cycloalkyl,

wherein the alkyl and cycloalkyl are optionally substituted with 1-3 substituents independently selected from:
halogen,
C₁₋₆alkyl,
or C₃₋₆cycloalkyl.

The present invention is also directed to compounds of Formula I or I-a or pharmaceutically acceptable salts thereof, wherein B is pyrimidine, optionally substituted with 1-3 substituents independently selected from:
halogen;
hydroxyl;
C₁₋₆alkyl;
C(O)OC₁₋₆alkyl;
C═O;
CN;
C₁₋₆alkoxy;
C₃₋₆cycloalkyl;
C(═O)—(O)ₙ—R′, wherein n is an integer from 0-3 and R′ is C₁₋₆alkyl or H;
5-6 membered heteroaryl ring containing 1-4 heteroatoms selected from nitrogen or oxygen;
C(═O)—R^{f}, wherein R^{f} is a 5-6 membered heteroaryl ring containing 1-4 heteroatoms independently selected from nitrogen or oxygen;
or haloC₁₋₆alkoxy;

wherein the alkyl moiety is optionally substituted with 1-3 substituents independently selected from:
halogen,
hydroxy,
C₁₋₆alkyl, or
C₁₋₆alkoxy.

The present invention is further directed to compounds of Formula I or I-a or pharmaceutically acceptable salts thereof, wherein B is 1,2,4-oxadiazol optionally substituted with 1-3 substituents independently selected from
C₁₋₆alkyl;
C₁₋₆alkoxy; or
C₃₋₆cycloalkyl,
wherein the alkyl, alkoxy and cycloalkyl are optionally substituted with 1-3 substituents independently selected from:
halogen,
C₁₋₆alkyl, or
C₃₋₆cycloalkyl.

In a particular embodiment of the present invention directed to compounds of formula I or I-a or pharmaceutically acceptable salts thereof, each R³ is independently selected from halogen which is further selected from F, Cl or Br, C₁₋₄alkyl, C₁₋₃alkoxy or C₃₋₆cycloalkyl.

In a particular embodiment of the present invention, B in compounds of formula I or I-a or pharmaceutically acceptable salts thereof is methoxymethyl-pyrimidine.

The present invention further encompasses compounds of Formula I or I-a or pharmaceutically acceptable salts thereof, wherein R² is halogen which is further selected from fluoro and chloro.

In other embodiments, in compounds of formula I or I-a or pharmaceutically acceptable salts thereof, R¹ is at the 4 position and is selected from: C₁₋₆alkyl; OC₁₋₆alkyl; C(O)C₁₋₆alkyl; C(O)C₃₋₆cycloalkyl; C(O)NHC₁₋₆alkyl; S(O)₀₋₂C₁₋₆alkyl; SO₂C₃₋₆cycloalkyl; SO₂NR^{b}R^{c}, wherein R^{b} and R^{c} are selected from H or C₁₋₆ alkyl; or a 5-6 membered heteroaryl ring containing 1-4 heteroatoms, 1-4 of which are nitrogen atoms, and 0-1 of which are O or S atoms, wherein the R¹ alkyl, cycloalkyl and heteroaryl moiety is optionally substituted with 1-3 substituents independently selected from: halogen; hydroxy; C₁₋₆alkyl or O—C₁₋₆alkyl.

In other embodiments, in compounds of formula I or I-a or pharmaceutically acceptable salts thereof, R¹ is at the 4 position and is selected from: CH₂CONR^{d}R^{e} wherein R^{d} and R^{e} are independently selected from H, C₁₋₆alkyl, C₃₋₆cycloalkyl, haloC₁₋₆alkyl, haloC₃₋₆cycloalkyl, C(O)NH₂, C₁₋₆alkoxy, or C₃₋₆cycloalkylC₁₋₆alkoxy; wherein R^{d} and R^{e}, if individually alkyl, alkoxy or C(O)NH₂, can together form a 4-6-membered saturated heterocyclic ring having 1 nitrogen atom which 4-6-membered ring may be optionally substituted with 1-3 substituents independently selected from halogen, hydroxy, oxo. C₁₋₆alkyl, C₁₋₆alkoxy; or CO₂C₁₋₆alkyl.

In one embodiment, R¹ in compounds of formula I or I-a or pharmaceutically acceptable salts thereof is methylsulfonyl.

In another embodiment, the compound of formula I is a compound selected from a compound within the following table:

| Example | Compound Name |
|---|---|
| 6 | 5-methyl-2-(4-((1R,2S)-2-(((4- |
| | (methylsulfonyl)benzyl)oxy)methyl)cyclopropyl)piperidin-1-yl)pyrimidine |
| 8 | 5-ethyl-2-(4-((1R,2S)-2-(((2-fluoro-4- |
| | (methylsulfonyl)benzyl)oxy)methyl)cyclopropyl)piperidin-1-yl)pyrimidine |
| 10 | 5-ethyl-2-(4-((1R,2S)-2-(((4- |
| | (methylsulfonyl)benzyl)oxy)methyl)cyclopropyl)piperidin-1-yl)pyrimidine |
| 17 | 1-methylcyclopropyl 4-((1R,2S)-2-(((3-fluoro-4- |
| | (methylsulfonyl)benzyl)oxy)methyl)cyclopropyl)piperidine-1-carboxylate |
| 18 | 1-methylcyclopropyl 4-((1R,2S)-2-(((4- |
| | (methylsulfonyl)benzyl)oxy)methyl)cyclopropyl)piperidine-1-carboxylate |
| 21 | 3-isopropyl-5-(4-((1R,2S)-2-((4- |
| | (methylsulfonyl)benzyloxy)methyl)cyclopropyl)piperidin-1-yl)-1,2,4-oxadiazole |
| 22 | 5-(4-((1R,2S)-2-(((3-fluoro-4- |
| | (methylsulfonyl)benzyl)oxy)methyl)cyclopropyl)piperidin-1-yl)-3-isopropyl-1,2,4- |
| | oxadiazole |
| 82 | 1-(azetidin-1-yl)-2-(2,6-difluoro-4-(2-((1S,2S)-2-(1-(3-isopropyl-1,2,4-oxadiazol-5- |
| | yl)piperidin-4-yl)cyclopropyl)ethoxy)phenyl)ethanone |
| 95 | 1-methylcyclopropyl 4-((1R,2R)-2-(2-(4-(2-(azetidin-1-yl)-2-oxoethyl)-3- |
| | fluorophenoxy)ethyl)cyclopropyl)piperidine-1-carboxylate |
| 103 | 1-methylcyclopropyl 4-((1R,2R)-2-(2-(4-(2-(azetidin-1-yl)-2-oxoethyl)-3,5- |
| | difluorophenoxy)ethyl)cyclopropyl)piperidine-1-carboxylate |

or a pharmaceutically acceptable salt thereof.

For compounds of formula I or a pharmaceutically acceptable salt thereof, the cyclopropyl ring is the trans cyclopropyl isomer.

Yet another aspect of the invention that is of interest relates to compounds of formula I or I-a, as well as the pharmaceutically acceptable salts thereof, selected from:

| Example | Compound Name |
|---|---|
| 1 | 5-chloro-2-(4-((1R,2S)-2-(((5-(methylsulfonyl)pyridin-2- |
| | yl)methoxy)methyl)cyclopropyl)piperidin-1-yl)pyrimidine |
| 2 | 5-chloro-2-(4-((1R,2S)-2-(((4- |
| | (methylsulfonyl)benzyl)oxy)methyl)cyclopropyl)piperidin-1-yl)pyrimidine |
| 3 | 5-chloro-2-(4-((1R,2S)-2-(((4- |
| | (ethylsulfonyl)benzyl)oxy)methyl)cyclopropyl)piperidin-1-yl)pyrimidine |
| 4 | 5-chloro-2-(4-((1R,2S)-2-(((2-fluoro-4- |
| | (methylsulfonyl)benzyl)oxy)methyl)cyclopropyl)piperidin-1-yl)pyrimidine |
| 5 | 5-chloro-2-(4-((1R,2S)-2-(((3-fluoro-4- |
| | (methylsulfonyl)benzyl)oxy)methyl)cyclopropyl)piperidin-1-yl)pyrimidine |
| 6 | 5-methyl-2-(4-((1R,2S)-2-(((4- |
| | (methylsulfonyl)benzyl)oxy)methyl)cyclopropyl)piperidin-1-yl)pyrimidine |
| 7 | 2-(4-((1R,2S)-2-(((4-(ethylsulfonyl)benzyl)oxy)methyl)cyclopropyl)piperidin-1-yl)- |
| | 5-methylpyrimidine |
| 8 | 5-ethyl-2-(4-((1R,2S)-2-(((2-fluoro-4- |
| | (methylsulfonyl)benzyl)oxy)methyl)cyclopropyl)piperidin-1-yl)pyrimidine |
| 9 | 5-ethyl-2-(4-((1R,2S)-2-(((4- |
| | (ethylsulfonyl)benzyl)oxy)methyl)cyclopropyl)piperidin-1-yl)pyrimidine |
| 10 | 5-ethyl-2-(4-((1R,2S)-2-(((4- |
| | (methylsulfonyl)benzyl)oxy)methyl)cyclopropyl)piperidin-1-yl)pyrimidine |
| 11 | 2-(4-((1R,2S)-2-(((2-fluoro-4- |
| | (methylsulfonyl)benzyl)oxy)methyl)cyclopropyl)piperidin-1-yl)-5- |
| | (methoxymethyl)pyrimidine |
| 12 | 5-(methoxymethyl)-2-(4-((1R,2S)-2-(((4- |
| | (methylsulfonyl)benzyl)oxy)methyl)cyclopropyl)piperidin-1-yl)pyrimidine |
| 13 | 2-(4-((1R,2S)-2-(((4-(ethylsulfonyl)benzyl)oxy)methyl)cyclopropyl)piperidin-1-yl)- |
| | 5-(methoxymethyl)pyrimidine |
| 14 | 1-methylcyclopropyl 4-((1R,2S)-2-((2-fluoro-4- |
| | (methylsulfonyl)benzyloxy)methyl)cyclopropyl)piperidine-1-carboxylate |
| 15 | cyclopropyl 4-((1R,2S)-2-(((3-fluoro-4- |
| | (methylsulfonyl)benzyl)oxy)methyl)cyclopropyl)piperidine-1-carboxylate |
| 16 | cyclopropyl 4-((1R,2S)-2-(((4- |
| | (methylsulfonyl)benzyl)oxy)methyl)cyclopropyl)piperidine-1-carboxylate |
| 17 | 1-methylcyclopropyl 4-((1R,2S)-2-(((3-fluoro-4- |
| | (methylsulfonyl)benzyl)oxy)methyl)cyclopropyl)piperidine-1-carboxylate |
| 18 | 1-methylcyclopropyl 4-((1R,2S)-2-(((4- |
| | (methylsulfonyl)benzyl)oxy)methyl)cyclopropyl)piperidine-1-carboxylate |
| 19 | isopropyl 4-((1R,2S)-2-(((4- |
| | (methylsulfonyl)benzyl)oxy)methyl)cyclopropyl)piperidine-1-carboxylate |
| 20 | isopropyl 4-((1R,2S)-2-(((3-fluoro-4- |
| | (methylsulfonyl)benzyl)oxy)methyl)cyclopropyl)piperidine-1-carboxylate |
| 21 | 3-isopropyl-5-(4-((1R,2S)-2-((4- |
| | (methylsulfonyl)benzyloxy)methyl)cyclopropyl)piperidin-1-yl)-1,2,4-oxadiazole |
| 22 | 5-(4-((1R,2S)-2-(((3-fluoro-4- |
| | (methylsulfonyl)benzyl)oxy)methyl)cyclopropyl)piperidin-1-yl)-3-isopropyl-1,2,4- |
| | oxadiazole |
| 23 | 1-methylcyclopropyl 4-((1R,2S)-2-((4-(2-(azetidin-1-yl)-2-oxoethyl)-2- |
| | fluorobenzyloxy)methyl)cyclopropyl)piperidine-1-carboxylate |
| 24 | 5-chloro-2-(4-((1S,2S)-2-(2-(3-fluoro-4- |
| | (methylsulfonyl)phenoxy)ethyl)cyclopropyl)piperidin-1-yl)pyrimidine |
| 25 | 5-chloro-2-(4-((1R,2R)-2-(2-(3-fluoro-4- |
| | (methylsulfonyl)phenoxy)ethyl)cyclopropyl)piperidin-1-yl)pyrimidine |
| 26 | 5-ethyl-2-(4-((1S,2S)-2-(2-(2-fluoro-4- |
| | (methylsulfonyl)phenoxy)ethyl)cyclopropyl)piperidin-1-yl)pyrimidine |
| 27 | 5-ethyl-2-(4-((1R,2R)-2-(2-(3-fluoro-4- |
| | (methylsulfonyl)phenoxy)ethyl)cyclopropyl)piperidin-1-yl)pyrimidine |
| 28 | 5-ethyl-2-(4-((1R,2R)-2-(2-(2-fluoro-4- |
| | (methylsulfonyl)phenoxy)ethyl)cyclopropyl)piperidin-1-yl)pyrimidine |
| 29 | 5-chloro-2-(4-((1S,2S)-2-(2-(2-fluoro-4- |
| | (methylsulfonyl)phenoxy)ethyl)cyclopropyl)piperidin-1-yl)pyrimidine |
| 30 | 5-chloro-2-(4-((1R,2R)-2-(2-(2-fluoro-4- |
| | (methylsulfonyl)phenoxy)ethyl)cyclopropyl)piperidin-1-yl)pyrimidine |
| 31 | 5-ethyl-2-(4-((1S,2S)-2-(2-(3-fluoro-4- |
| | (methylsulfonyl)phenoxy)ethyl)cyclopropyl)piperidin-1-yl)pyrimidine |
| 32 | 5-ethyl-2-(4-((1S,2S)-2-(2-(4-(ethylsulfonyl)-2- |
| | fluorophenoxy)ethyl)cyclopropyl)piperidin-1-yl)pyrimidine |
| 33 | 2-(4-((1R,2R)-2-(2-(3-fluoro-4- |
| | (methylsulfonyl)phenoxy)ethyl)cyclopropyl)piperidin-1-yl)-5- |
| | (methoxymethyl)pyrimidine |
| 34 | 1-(azetidin-1-yl)-2-(4-(2-((1R,2R)-2-(1-(5-chloropyrimidin-2-yl)piperidin-4- |
| | yl)cyclopropyl)ethoxy)-2-fluorophenyl)ethanone |
| 35 | 1-(azetidin-1-yl)-2-(4-(2-((1R,2R)-2-(1-(5-chloropyrimidin-2-yl)piperidin-4- |
| | yl)cyclopropyl)ethoxy)-3-fluorophenyl)ethanone |
| 36 | 1-(azetidin-1-yl)-2-(4-(2-((1S,2S)-2-(1-(5-ethylpyrimidin-2-yl)piperidin-4- |
| | yl)cyclopropyl)ethoxy)-2-fluorophenyl)ethanone |
| 37 | 5-chloro-2-(4-((1R,2R)-2-(2-(3-fluoro-4-(1H-tetrazol-1- |
| | yl)phenoxy)ethyl)cyclopropyl)piperidin-1-yl)pyrimidine |
| 38 | 5-ethyl-2-(4-((1R,2R)-2-(2-(3-fluoro-4-(1H-tetrazol-1- |
| | yl)phenoxy)ethyl)cyclopropyl)piperidin-1-yl)pyrimidine |
| 39 | 5-ethyl-2-(4-((1S,2S)-2-(2-(3-fluoro-4-(1H-tetrazol-1- |
| | yl)phenoxy)ethyl)cyclopropyl)piperidin-1-yl)pyrimidine |
| 40 | 5-ethyl-2-(4-((1S,2S)-2-(2-(2-fluoro-4-(1H-tetrazol-1- |
| | yl)phenoxy)ethyl)cyclopropyl)piperidin-1-yl)pyrimidine |
| 41 | 5-chloro-2-(4-((1S,2S)-2-(2-(2-fluoro-4-(1H-tetrazol-1- |
| | yl)phenoxy)ethyl)cyclopropyl)piperidin-1-yl)pyrimidine |
| 42 | 1-(azetidin-1-yl)-2-(4-(2-((1S,2S)-2-(1-(5-chloropyrimidin-2-yl)piperidin-4- |
| | yl)cyclopropyl)ethoxy)-2-fluorophenyl)ethanone |
| 43 | 1-(4-(2-((1R,2R)-2-(1-(5-ethylpyrimidin-2-yl)piperidin-4-yl)cyclopropyl)ethoxy)-2- |
| | fluorophenyl)imidazolidin-2-one |
| 44 | 1-(azetidin-1-yl)-2-(4-(2-((1R,2R)-2-(1-(5-ethylpyrimidin-2-yl)piperidin-4- |
| | yl)cyclopropyl)ethoxy)-3-fluorophenyl)ethanone |
| 45 | 1-(azetidin-1-yl)-2-(4-(2-((1R,2R)-2-(1-(5-ethylpyrimidin-2-yl)piperidin-4- |
| | yl)cyclopropyl)ethoxy)-2-fluorophenyl)ethanone |
| 46 | 2-(4-(2-((1S,2S)-2-(1-(5-ethylpyrimidin-2-yl)piperidin-4-yl)cyclopropyl)ethoxy)-2- |
| | fluorophenyl)-N,N-dimethylacetamide |
| 47 | 2-(4-(2-((1R,2R)-2-(1-(5-ethylpyrimidin-2-yl)piperidin-4-yl)cyclopropyl)ethoxy)-2- |
| | fluorophenyl)-N,N-dimethylacetamide |
| 48 | 1-(azetidin-1-yl)-2-(2-fluoro-4-(2-((1S,2S)-2-(1-(5-(methoxymethyl)pyrimidin-2- |
| | yl)piperidin-4-yl)cyclopropyl)ethoxy)phenyl)ethanone |
| 49 | 2-(4-(2-((1S,2S)-2-(1-(5-ethylpyrimidin-2-yl)piperidin-4-yl)cyclopropyl)ethoxy)- |
| | 2,6-difluorophenyl)-N,N-dimethylacetamide |
| 50 | 1-(azetidin-1-yl)-2-(4-(2-((1S,2S)-2-(1-(5-ethylpyrimidin-2-yl)piperidin-4- |
| | yl)cyclopropyl)ethoxy)-2,6-difluorophenyl)ethanone |
| 51 | 1-(azetidin-1-yl)-2-(2-fluoro-4-(2-((1R,2R)-2-(1-(5-(methoxymethyl)pyrimidin-2- |
| | yl)piperidin-4-yl)cyclopropyl)ethoxy)phenyl)ethanone |
| 52 | 2-(2-fluoro-4-(2-((1S,2S)-2-(1-(5-(methoxymethyl)pyrimidin-2-yl)piperidin-4- |
| | yl)cyclopropyl)ethoxy)phenyl)-N,N-dimethylacetamide |
| 53 | 2-(2-fluoro-4-(2-((1R,2R)-2-(1-(5-(methoxymethyl)pyrimidin-2-yl)piperidin-4- |
| | yl)cyclopropyl)ethoxy)phenyl)-N,N-dimethylacetamide |
| 54 | 1-(azetidin-1-yl)-2-(4-(2-((1R,2R)-2-(1-(5-ethylpyrimidin-2-yl)piperidin-4- |
| | yl)cyclopropyl)ethoxy)-2,6-difluorophenyl)ethanone |
| 55 | 2-(4-(2-((1R,2R)-2-(1-(5-ethylpyrimidin-2-yl)piperidin-4-yl)cyclopropyl)ethoxy)- |
| | 2,6-difluorophenyl)-N,N-dimethylacetamide |
| 56 | 1-(azetidin-1-yl)-2-(2,6-difluoro-4-(2-((1R,2R)-2-(1-(5-(methoxymethyl)pyrimidin- |
| | 2-yl)piperidin-4-yl)cyclopropyl)ethoxy)phenyl)ethanone |
| 57 | 2-(2,6-difluoro-4-(2-((1R,2R)-2-(1-(5-(methoxymethyl)pyrimidin-2-yl)piperidin-4- |
| | yl)cyclopropyl)ethoxy)phenyl)-N,N-dimethylacetamide |
| 58 | 5-chloro-2-(4-((1R,2R)-2-(2-(2-fluoro-4-(1H-tetrazol-1- |
| | yl)phenoxy)ethyl)cyclopropyl)piperidin-1-yl)pyrimidine |
| 59 | 5-chloro-2-(4-((1S,2S)-2-(2-(3-fluoro-4-(1H-tetrazol-1- |
| | yl)phenoxy)ethyl)cyclopropyl)piperidin-1-yl)pyrimidine |
| 60 | 5-ethyl-2-(4-((1R,2R)-2-(2-(2-fluoro-4-(1H-tetrazol-1- |
| | yl)phenoxy)ethyl)cyclopropyl)piperidin-1-yl)pyrimidine |
| 61 | 1-(4-(2-((1R,2R)-2-(1-(5-chloropyrimidin-2-yl)piperidin-4-yl)cyclopropyl)ethoxy)- |
| | 2-fluorophenyl)imidazolidin-2-one |
| 62 | 1-(azetidin-1-yl)-2-(4-(2-((1S,2S)-2-(1-(5-ethylpyrimidin-2-yl)piperidin-4- |
| | yl)cyclopropyl)ethoxy)-3-fluorophenyl)ethanone |
| 63 | 1-(azetidin-1-yl)-2-(3-fluoro-4-(2-((1R,2R)-2-(1-(5-(methoxymethyl)pyrimidin-2- |
| | yl)piperidin-4-yl)cyclopropyl)ethoxy)phenyl)ethanone |
| 64 | 2-(4-(2-((1S,2S)-2-(1-(5-chloropyrimidin-2-yl)piperidin-4-yl)cyclopropyl)ethoxy)- |
| | 2-fluorophenyl)-5-methyl-1,3,4-oxadiazole |
| 65 | 5-(4-((1S,2S)-2-(2-(3-fluoro-4- |
| | (methylsulfonyl)phenoxy)ethyl)cyclopropyl)piperidin-1-yl)-3-isopropyl-1,2,4- |
| | oxadiazole |
| 66 | 5-(4-((1S,2S)-2-(2-(2-fluoro-4- |
| | (methylsulfonyl)phenoxy)ethyl)cyclopropyl)piperidin-1-yl)-3-isopropyl-1,2,4- |
| | oxadiazole |
| 67 | 5-(4-((1S,2S)-2-(2-(2-fluoro-4-(1H-tetrazol-1- |
| | yl)phenoxy)ethyl)cyclopropyl)piperidin-1-yl)-3-isopropyl-1,2,4-oxadiazole |
| 68 | 5-(4-((1R,2R)-2-(2-(2-fluoro-4-(1H-tetrazol-1- |
| | yl)phenoxy)ethyl)cyclopropyl)piperidin-1-yl)-3-isopropyl-1,2,4-oxadiazole |
| 69 | 1-(azetidin-1-yl)-2-(2-fluoro-4-(2-((1S,2S)-2-(1-(3-isopropyl-1,2,4-oxadiazol-5- |
| | yl)piperidin-4-yl)cyclopropyl)ethoxy)phenyl)ethanone |
| 70 | 1-(azetidin-1-yl)-2-(3-fluoro-4-(2-((1S,2S)-2-(1-(3-isopropyl-1,2,4-oxadiazol-5- |
| | yl)piperidin-4-yl)cyclopropyl)ethoxy)phenyl)ethanone |
| 71 | 5-(4-((1S,2S)-2-(2-(4-(ethylsulfonyl)-2-fluorophenoxy)ethyl)cyclopropyl)piperidin- |
| | 1-yl)-3-isopropyl-1,2,4-oxadiazole |
| 72 | 1-(azetidin-1-yl)-2-(2-fluoro-4-(2-((1R,2R)-2-(1-(3-isopropyl-1,2,4-oxadiazol-5- |
| | yl)piperidin-4-yl)cyclopropyl)ethoxy)phenyl)ethanone |
| 73 | 1-(azetidin-1-yl)-2-(2-fluoro-4-(2-((1R,2R)-2-(1-(3-(methoxymethyl)-1,2,4- |
| | oxadiazol-5-yl)piperidin-4-yl)cyclopropyl)ethoxy)phenyl)ethanone |
| 74 | 1-(azetidin-1-yl)-2-(3-fluoro-4-(2-((1R,2R)-2-(1-(3-isopropyl-1,2,4-oxadiazol-5- |
| | yl)piperidin-4-yl)cyclopropyl)ethoxy)phenyl)ethanone |
| 75 | 2-(2-fluoro-4-(2-((1R,2R)-2-(1-(3-isopropyl-1,2,4-oxadiazol-5-yl)piperidin-4- |
| | yl)cyclopropyl)ethoxy)phenyl)-N,N-dimethylacetamide |
| 76 | 2-(2-fluoro-4-(2-((1S,2S)-2-(1-(3-isopropyl-1,2,4-oxadiazol-5-yl)piperidin-4- |
| | yl)cyclopropyl)ethoxy)phenyl)-N,N-dimethylacetamide |
| 77 | 2-(2,6-difluoro-4-(2-((1S,2S)-2-(1-(3-isopropyl-1,2,4-oxadiazol-5-yl)piperidin-4- |
| | yl)cyclopropyl)ethoxy)phenyl)-N,N-dimethylacetamide |
| 78 | 2-(2,6-difluoro-4-(2-((1S,2S)-2-(1-(3-(methoxymethyl)-1,2,4-oxadiazol-5- |
| | yl)piperidin-4-yl)cyclopropyl)ethoxy)phenyl)-N,N-dimethylacetamide |
| 79 | 1-(azetidin-1-yl)-2-(2,6-difluoro-4-(2-((1R,2R)-2-(1-(3-isopropyl-1,2,4-oxadiazol- |
| | 5-yl)piperidin-4-yl)cyclopropyl)ethoxy)phenyl)ethanone |
| 80 | 1-(azetidin-1-yl)-2-(2,6-difluoro-4-(2-((1R,2R)-2-(1-(3-(methoxymethyl)-1,2,4- |
| | oxadiazol-5-yl)piperidin-4-yl)cyclopropyl)ethoxy)phenyl)ethanone |
| 81 | 2-(2,6-difluoro-4-(2-((1R,2R)-2-(1-(3-isopropyl-1,2,4-oxadiazol-5-yl)piperidin-4- |
| | yl)cyclopropyl)ethoxy)phenyl)-N,N-dimethylacetamide |
| 82 | 1-(azetidin-1-yl)-2-(2,6-difluoro-4-(2-((1S,2S)-2-(1-(3-isopropyl-1,2,4-oxadiazol-5- |
| | yl)piperidin-4-yl)cyclopropyl)ethoxy)phenyl)ethanone |
| 83 | 1-(azetidin-1-yl)-2-(2,6-difluoro-4-(2-((1S,2S)-2-(1-(3-(methoxymethyl)-1,2,4- |
| | oxadiazol-5-yl)piperidin-4-yl)cyclopropyl)ethoxy)phenyl)ethanone |
| 84 | 2-(2,6-difluoro-4-(2-((1R,2R)-2-(1-(3-(methoxymethyl)-1,2,4-oxadiazol-5- |
| | yl)piperidin-4-yl)cyclopropyl)ethoxy)phenyl)-N,N-dimethylacetamide |
| 85 | 5-(4-((1R,2R)-2-(2-(2-fluoro-4- |
| | (methylsulfonyl)phenoxy)ethyl)cyclopropyl)piperidin-1-yl)-3-isopropyl-1,2,4- |
| | oxadiazole |
| 86 | 5-(4-((1R,2R)-2-(2-(3-fluoro-4-(1H-tetrazol-1- |
| | yl)phenoxy)ethyl)cyclopropyl)piperidin-1-yl)-3-isopropyl-1,2,4-oxadiazole |
| 87 | 5-(4-((1R,2R)-2-(2-(3-fluoro-4- |
| | (methylsulfonyl)phenoxy)ethyl)cyclopropyl)piperidin-1-yl)-3-isopropyl-1,2,4- |
| | oxadiazole |
| 88 | 1-(3-fluoro-4-(2-((1S,2S)-2-(1-(3-isopropyl-1,2,4-oxadiazol-5-yl)piperidin-4- |
| | yl)cyclopropyl)ethoxy)phenyl)imidazolidin-2-one |
| 89 | 5-(4-((1S,2S)-2-(2-(2-fluoro-4-(1H-tetrazol-1- |
| | yl)phenoxy)ethyl)cyclopropyl)piperidin-1-yl)-3-(methoxymethyl)-1,2,4-oxadiazole |
| 90 | 5-(4-((1R,2R)-2-(2-(2-fluoro-4-(1H-tetrazol-1- |
| | yl)phenoxy)ethyl)cyclopropyl)piperidin-1-yl)-3-(methoxymethyl)-1,2,4-oxadiazole |
| 91 | 1-(azetidin-1-yl)-2-(2-fluoro-4-(2-((1S,2S)-2-(1-(3-(methoxymethyl)-1,2,4- |
| | oxadiazol-5-yl)piperidin-4-yl)cyclopropyl)ethoxy)phenyl)ethanone |
| 92 | 5-(4-((1R,2R)-2-(2-(4-(ethylsulfonyl)-2-fluorophenoxy)ethyl)cyclopropyl)piperidin- |
| | 1-yl)-3-isopropyl-1,2,4-oxadiazole |
| 93 | 2-(2-fluoro-4-(2-((1R,2R)-2-(1-(3-(methoxymethyl)-1,2,4-oxadiazol-5-yl)piperidin- |
| | 4-yl)cyclopropyl)ethoxy)phenyl)-N,N-dimethylacetamide |
| 94 | 1-methylcyclopropyl 4-((1S,2S)-2-(2-(3-fluoro-4- |
| | (methylsulfonyl)phenoxy)ethyl)cyclopropyl)piperidine-1-carboxylate |
| 95 | 1-methylcyclopropyl 4-((1R,2R)-2-(2-(4-(2-(azetidin-1-yl)-2-oxoethyl)-3- |
| | fluorophenoxy)ethyl)cyclopropyl)piperidine-1-carboxylate |
| 96 | 1-methylcyclopropyl 4-((1S,2S)-2-(2-(2-fluoro-4-(1H-tetrazol-1- |
| | yl)phenoxy)ethyl)cyclopropyl)piperidine-1-carboxylate |
| 97 | 1-methylcyclopropyl 4-((1S,2S)-2-(2-(4-(2-(azetidin-1-yl)-2-oxoethyl)-3- |
| | fluorophenoxy)ethyl)cyclopropyl)piperidine-1-carboxylate |
| 98 | 1-methylcyclopropyl 4-((1R,2R)-2-(2-(4-(2-(azetidin-1-yl)-2-oxoethyl)-2- |
| | fluorophenoxy)ethyl)cyclopropyl)piperidine-1-carboxylate |
| 99 | 1-methylcyclopropyl 4-((1S,2S)-2-(2-(4-(2-(dimethylamino)-2-oxoethyl)-3- |
| | fluorophenoxy)ethyl)cyclopropyl)piperidine-1-carboxylate |
| 100 | 1-methylcyclopropyl 4-((1R,2R)-2-(2-(4-(2-(dimethylamino)-2-oxoethyl)-3- |
| | fluorophenoxy)ethyl)cyclopropyl)piperidine-1-carboxylate |
| 101 | 1-methylcyclopropyl 4-((1S,2S)-2-(2-(4-(2-(azetidin-1-yl)-2-oxoethyl)-3,5- |
| | difluorophenoxy)ethyl)cyclopropyl)piperidine-1-carboxylate |
| 102 | 1-methylcyclopropyl 4-((1S,2S)-2-(2-(4-(2-(dimethylamino)-2-oxoethyl)-3,5- |
| | difluorophenoxy)ethyl)cyclopropyl)piperidine-1-carboxylate |
| 103 | 1-methylcyclopropyl 4-((1R,2R)-2-(2-(4-(2-(azetidin-1-yl)-2-oxoethyl)-3,5- |
| | difluorophenoxy)ethyl)cyclopropyl)piperidine-1-carboxylate |
| 104 | 1-methylcyclopropyl 4-((1R,2R)-2-(2-(4-(2-(dimethylamino)-2-oxoethyl)-3,5- |
| | difluorophenoxy)ethyl)cyclopropyl)piperidine-1-carboxylate |
| 105 | 1-methylcyclopropyl 4-((1S,2S)-2-(2-(3-fluoro-4-(1H-tetrazol-1- |
| | yl)phenoxy)ethyl)cyclopropyl)piperidine-1-carboxylate |
| 106 | 1-methylcyclopropyl 4-((1R,2R)-2-(2-(3-fluoro-4-(1H-tetrazol-1- |
| | yl)phenoxy)ethyl)cyclopropyl)piperidine-1-carboxylate |
| 107 | 1-methylcyclopropyl 4-((1R,2R)-2-(2-(2-fluoro-4-(1H-tetrazol-1- |
| | yl)phenoxy)ethyl)cyclopropyl)piperidine-1-carboxylate |
| 108 | 1-methylcyclopropyl 4-((1S,2S)-2-(2-(4-(2-(azetidin-1-yl)-2-oxoethyl)-2- |
| | fluorophenoxy)ethyl)cyclopropyl)piperidine-1-carboxylate |
| 109 | 5-chloro-2-(4-((1R,2R)-2-(2-(5-(methylsulfonyl)pyridin-2- |
| | yloxy)ethyl)cyclopropyl)piperidin-1-yl)pyrimidine |
| 110 | 3-isopropyl-5-(4-((1R,2R)-2-(2-(5-(methylsulfonyl)pyridin-2- |
| | yloxy)ethyl)cyclopropyl)piperidin-1-yl)-1,2,4-oxadiazole |
| 111 | 5-(4-((1S ,2S)-2-(2-((5-(ethylsulfonyl)pyridin-2-yl)oxy)ethyl)cyclopropyl)piperidin- |
| | 1-yl)-3-isopropyl-1,2,4-oxadiazole |
| 112 | 3-cyclopropyl-5-(4-((1S,2S)-2-(2-((5-(methylsulfonyl)pyridin-2- |
| | yl)oxy)ethyl)cyclopropyl)piperidin-1-yl)-1,2,4-oxadiazole |
| 113 | 5-(4-((1S,2S)-2-(2-((5-(methylsulfonyl)pyridin-2- |
| | yl)oxy)ethyl)cyclopropyl)piperidin-1-yl)-3-propyl-1,2,4-oxadiazole |
| 114 | 3-cyclobutyl-5-(4-((1S,2S)-2-(2-((5-(methylsulfonyl)pyridin-2- |
| | yl)oxy)ethyl)cyclopropyl)piperidin-1-yl)-1,2,4-oxadiazole |
| 115 | 1-(azetidin-1-yl)-2-(6-(2-((1R,2R)-2-(1-(5-ethylpyrimidin-2-yl)piperidin-4- |
| | yl)cyclopropyl)ethoxy)pyridin-3-yl)ethanone |
| 116 | 1-(azetidin-1-yl)-2-(6-(2-((1S,2S)-2-(1-(5-ethylpyrimidin-2-yl)piperidin-4- |
| | yl)cyclopropyl)ethoxy)pyridin-3-yl)ethanone |
| 117 | 2-(2-(2-((1R,2R)-2-(1-(5-ethylpyrimidin-2-yl)piperidin-4-yl)cyclopropyl)ethoxy)- |
| | 4,6-difluorophenyl)-N,N-dimethylacetamide |
| 118 | 1-(azetidin-1-yl)-2-(2-(2-((1S,2S)-2-(1-(5-ethylpyrimidin-2-yl)piperidin-4- |
| | yl)cyclopropyl)ethoxy)-4,6-difluorophenyl)ethanone |
| 119 | 1-(azetidin-1-yl)-2-(2-(2-((1R,2R)-2-(1-(5-ethylpyrimidin-2-yl)piperidin-4- |
| | yl)cyclopropyl)ethoxy)-4,6-difluorophenyl)ethanone |
| 120 | 1-methylcyclopropyl 4-((1S,2S)-2-(2-(5-(2-(azetidin-1-yl)-2-oxoethyl)pyridin-2- |
| | yloxy)ethyl)cyclopropyl)piperidine-1-carboxylate |
| 121 | 1-(azetidin-1-yl)-2-(6-(2-((1S,2S)-2-(1-(3-isopropyl-1,2,4-oxadiazol-5-yl)piperidin- |
| | 4-yl)cyclopropyl)ethoxy)pyridin-3-yl)ethanone |
| 122 | 2-(2,4-difluoro-6-(2-((1R,2R)-2-(1-(3-isopropyl-1,2,4-oxadiazol-5-yl)piperidin-4- |
| | yl)cyclopropyl)ethoxy)phenyl)-N,N-dimethylacetamide |
| 123 | N-(2-((1R,2S)-2-(1-(5-chloropyrimidin-2-yl)piperidin-4-yl)cyclopropyl)ethyl)-5- |
| | (1H-tetrazol-1-yl)pyrimidin-2-amine |
| 124 | N-(2-((1R,2S)-2-(1-(5-(methoxymethyl)pyrimidin-2-yl)piperidin-4- |
| | yl)cyclopropyl)ethyl)-5-(1H-tetrazol-1-yl)pyrimidin-2-amine |
| 125 | N-(2-((1R,2S)-2-(1-(5-ethylpyrimidin-2-yl)piperidin-4-yl)cyclopropyl)ethyl)-5-(1H- |
| | tetrazol-1-yl)pyrimidin-2-amine |
| 126 | N-(2-((1S,2R)-2-(1-(3-isopropyl-1,2,4-oxadiazol-5-yl)piperidin-4- |
| | yl)cyclopropyl)ethyl)-5-(1H-tetrazol-1-yl)pyrimidin-2-amine |
| 127 | (1S,2R)-2-(1-(5-chloropyrimidin-2-yl)piperidin-4-yl)-1-(2-(4- |
| | (methylsulfonyl)phenoxy)ethyl)cyclopropanecarbonitrile |
| 128 | N-(2-((1R,2S)-2-(1-(5-chloropyrimidin-2-yl)piperidin-4-yl)cyclopropyl)ethyl)-4- |
| | (methylsulfonyl)aniline |

The present invention also relates to pharmaceutical compositions comprising compounds of formula I or a pharmaceutically acceptable salt thereof, and a pharmaceutically acceptable carrier.

Additionally, the present invention relates to use of a compound of Formula I or a pharmaceutically acceptable salt thereof, in the manufacture of a medicament for use in treating a condition selected from the group consisting of obesity and diabetes.

The present invention relates to the use of a compound of Formula I or a pharmaceutically acceptable salt thereof, in the treatment of diabetes.

The present invention further relates to a method for the treatment of a condition selected from obesity or diabetes comprising administering to an individual a pharmaceutical composition comprising the compound of Formula I.

Another embodiment of the present invention includes a method of treating a condition selected from: (1) hyperglycemia, (2) impaired glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) Syndrome X, (21) hypertension or other conditions and disorders where insulin resistance is a component, in a mammalian patient in need of such treatment, comprising administering to the patient a compound of claim 1, or a pharmaceutically acceptable salt thereof, in an amount that is effective to treat said condition.

Yet another embodiment of the present invention include a method of treating a condition selected from: (1) hyperglycemia, (2) impaired glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) Syndrome X, (21) hypertension or other conditions and disorders where insulin resistance is a component, in a mammalian patient in need of such treatment, comprising administering to the patient a compound of Formula I, or a pharmaceutically acceptable salt thereof, and a compound selected from:
(a) DPP-IV inhibitors;
(b) insulin sensitizers selected from the group consisting of (i) PPAR agonists and (ii) biguanides;
(c) insulin and insulin mimetics;
(d) sulfonylureas and other insulin secretagogues;
(e) α-glucosidase inhibitors;
(f) glucagon receptor antagonists;
(g) GLP-1, GLP-1 mimetics, and GLP-1 receptor agonists;
(h) GIP, GIP mimetics, and GIP receptor agonists;

(i) PACAP, PACAP mimetics, and PACAP receptor 3 agonists;
(j) cholesterol lowering agents selected from
   (i) HMG-CoA reductase inhibitors, (ii) sequestrants, (iii) nicotinyl alcohol, nicotinic acid and salts thereof, (iv) PPARα agonists, (v) PPARα/γ dual agonists, (vi) inhibitors of cholesterol absorption, (vii) acyl CoA:cholesterol acyltransferase inhibitors, or (viii) anti-oxidants;
(k) PPARδ agonists;
(l) antiobesity compounds;
(m) ileal bile acid transporter inhibitors;
(n) anti-inflammatory agents excluding glucocorticoids;
(o) protein tyrosine phosphatase-1B (PTP-1B) inhibitors; or
(p) antihypertensives including those acting on the angiotensin or renin systems, such as angiotensin converting enzyme inhibitors, angiotensin II receptor antagonists or renin inhibitors, such as captopril, cilazapril, enalapril, fosinopril, lisinopril, quinapril, ramapril, zofenopril, candesartan, cilexetil, eprosartan, irbesartan, losartan, tasosartan, telmisartan, and valsartan, said compounds being administered to the patient in an amount that is effective to treat said condition.

The invention is described herein in detail using the terms defined below unless otherwise specified.

The language stating that “bonds x and y are in trans orientation in reference to one another” means that the structure:

if Formula I, is

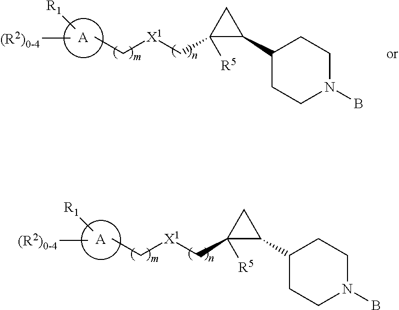

Or

if Formula I-a, is

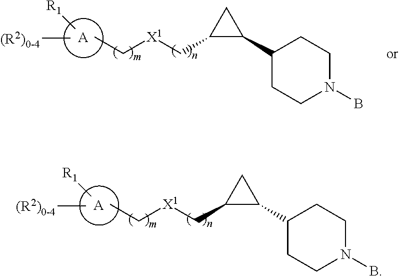

“Alkyl”, as well as other groups having the prefix “alk”, such as alkoxy, and the like, means carbon chains which may be linear or branched, or combinations thereof, containing the indicated number of carbon atoms. If no number is specified, 1-6 carbon atoms are intended for linear and 3-7 carbon atoms for branched alkyl groups. Examples of alkyl groups include methyl, ethyl, propyl, isopropyl, butyl, sec- and tert-butyl, pentyl, hexyl, heptyl, octyl, nonyl and the like.

As used herein, “cycloalkyl” means a saturated cyclic hydrocarbon radical having the number of carbon atoms designated. If no number of atoms is specified, 3-7 carbon atoms are intended, forming 1-3 carbocyclic rings that are fused. “Cycloalkyl” also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl and the like.

“Alkoxy” refers to an alkyl group linked to oxygen.

“Haloalkoxy” and “haloalkylO” are used interchangeably and refer to halo substituted alkyl groups linked through the oxygen atom. Haloalkoxy include mono-substituted as well as multiple halo substituted alkoxy groups, up to perhalo substituted alkoxy. For example, trifluoromethoxy is included.

“Haloalkyl” include mono-substituted as well as multiple halo substituted alkyl groups, up to perhalo substituted alkyl. For example, trifluoromethyl is included.

As used herein, “heterocycle” or “heterocyclic” refers to nonaromatic cyclic ring structures in which one or more atoms in the ring, the heteroatom(s), is an element other than carbon. Heteroatoms are typically O, S or N atoms.

“Heteroaryl” (HAR) unless otherwise specified, means an aromatic or partially aromatic heterocycle that contains at least one ring heteroatom selected from oxygen (“O”), sulfur (“S”) and nitrogen (“N”). Heteroaryls thus includes heteroaryls fused to other kinds of rings, such as aryls, cycloalkyls and heterocycles that are not aromatic. Examples of heteroaryl groups include: pyrrolyl or pyrrole, isoxazolyl or isoxazole, isothiazolyl or isothiazole, pyrazolyl or pyrazole, pyridyl, oxazolyl or oxazole, oxadiazolyl or oxadiazole, thiadiazolyl or thiadiazole, thiazolyl or thiazole, imidazolyl or imidazole, triazolyl or triazole, tetrazolyl or tetrazole, furyl, triazinyl, thienyl, pyrimidyl, benzisoxazolyl or benzisoxazole, benzoxazolyl or benzoazole, benzothiazolyl or benzothiazole, benzothiadiazolyl or benzothiadiazole, dihydrobenzofuranyl or dihydrobenzofurane, indolinyl or indoline, pyridazinyl or pyridazine, indazolyl or indazole, isoindolyl or isoindole, dihydrobenzothienyl, indolizinyl or indolizine, cinnolinyl or cinnoline, phthalazinyl or phthalazine, quinazolinyl or quinazoline, naphthyridinyl or naphthyridine, carbazolyl or carbazole, benzodioxolyl or benzodioxole, quinoxalinyl or quinoxaline, purinyl or purine, furazanyl or furazane, isobenzylfuranyl or isobenzylfurane, benzimidazolyl or benzimidazole, benzofuranyl or benzofurane, benzothienyl or benzothiene, quinolyl or quinoline, oxo-dihydroqunoline, indolyl or indole, oxindole, isoquinolyl or isoquinoline, dibenzofuranyl or dibenzofurane, and the like. For heterocyclic and heteroaryl groups, rings and ring systems containing from 5-15 atoms are included, forming 1-3 rings.

“Halogen” (Halo) includes fluorine, chlorine, bromine and iodine.

Unless expressly depicted or described otherwise, variables depicted in a structural formula with a “floating” bond, such as each of substituents R¹ and R², are permitted on any available carbon atom in the ring to which each is attached.

Substitution, where applicable, may be on any available carbon atom that results in a stable structure.

Also, number ranges where provided (e.g., 1-4, 0-3, etc.) expressly include each and every number in that range as a discrete embodiment.

In the compounds described herein, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of the formulas described herein. For example, different isotopic forms of hydrogen (H) include protium (¹H) and deuterium (²H). Protium is the predominant hydrogen isotope found in nature. Enriching for deuterium may yield certain therapeutic advantages, such as increasing in vivo half-life or reducing dosage requirements, or may provide a compound useful as a standard for characterization of biological samples. Isotopically-enriched compounds within the formulas described herein can be prepared without undue experimentation by conventional techniques well known to those skilled in the art or by processes analogous to those described in the Schemes and Examples herein using appropriate isotopically-enriched reagents and/or intermediates.

The individual tautomers of the compounds of the formulas described herein, as well as mixture thereof, are encompassed with compounds of the formulas described herein. Tautomers are defined as compounds that undergo rapid proton shifts from one atom of the compound to another atom of the compound. Some of the compounds described herein may exist as tautomers with different points of attachment of hydrogen. Such an example may be a ketone and its enol form known as keto-enol tautomers.

Compounds of the formulas described herein may be separated into diastereoisomeric pairs of enantiomers by, for example, fractional crystallization from a suitable solvent. The pair of enantiomers thus obtained may be separated into individual stereoisomers by conventional means, for example by the use of an optically active amine or acid as a resolving agent or on a chiral HPLC column.

Alternatively, any enantiomer of a compound of the formulas described herein may be obtained by stereospecific synthesis using optically pure starting materials or reagents of known configuration.

It is generally preferable to administer compounds of the present invention as enantiomerically pure formulations. Racemic mixtures can be separated into their individual enantiomers by any of a number of conventional methods. These include chiral chromatography, derivatization with a chiral auxiliary followed by separation by chromatography or crystallization, and fractional crystallization of diastereomeric salts.

Compounds described herein may contain an asymmetric center and may thus exist as enantiomers. Where the compounds according to the invention possess two or more asymmetric centers, they may additionally exist as diastereomers. When bonds to the chiral carbon are depicted as straight lines in the formulas of the invention, it is understood that both the (R) and (S) configurations of the chiral carbon, and hence both enantiomers and mixtures thereof, are embraced within the formulas. The present invention includes all such possible stereoisomers as substantially pure resolved enantiomers, racemic mixtures thereof, as well as mixtures of diastereomers. Except where otherwise specified, the formulae encompassing compounds of the present invention are shown without a definitive stereochemistry at certain positions. The present invention therefore may be understood to include all stereoisomers of compounds of Formula I and pharmaceutically acceptable salts thereof.

Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound of the general Formula I or Ia may be obtained by stereospecific synthesis using optically pure starting materials or reagents of known configuration.

Furthermore, some of the crystalline forms for compounds of the present invention may exist as polymorphs and as such are intended to be included in the present invention. In addition, some of the compounds of the instant invention may form solvates with water or common organic solvents. Solvates, and in particular, the hydrates of the compounds of the structural formulas described herein are also included in the present invention.

Compounds of the present invention are potent agonists of the GPR 119 receptor. These compounds and pharmaceutically acceptable salts thereof are modulators of the receptor known as GPR 119, and are therefore useful in the treatment of diseases that are modulated by GPR119 ligands and agonists. Many of these diseases are summarized below. Said compounds may be used for the manufacture of a medicament for treating one or more of diseases or conditions, including, without limitation:
(1) noninsulin dependent diabetes mellitus (type 2 diabetes);
(2) hyperglycemia;
(3) metabolic syndrome/syndrome X;
(4) obesity;
(5) ischemia and myocardial infarction;
(6) neurological disorders such as Alzheimer's disease, schizophrenia, and impaired cognition;
(5) hypercholesterolemia;
(6) hypertriglyceridemia (elevated levels of triglyceride-rich-lipoproteins);
(7) mixed or diabetic dyslipidemia;
(8) low HDL cholesterol;
(9) high LDL cholesterol;
(10) Hyperapobetalipoproteinemia; and
(11) atherosclerosis.

Because the compounds are agonists of the GPR119 receptor, the compounds will be useful in therapy, for lowering glucose, lipids, and insulin resistance in diabetic patients and in non-diabetic patients who have impaired glucose tolerance and/or are in a pre-diabetic condition. The compounds are useful to ameliorate hyperinsulinemia, which often occurs in diabetic or pre-diabetic patients, by modulating the swings in the level of serum glucose that often occurs in these patients. The compounds are useful for treating or reducing insulin resistance. The compounds are useful for treating or preventing gestational diabetes.

Additionally, by keeping hyperglycemia under control, the compounds are useful to delay or for preventing vascular restenosis and diabetic retinopathy.

The compounds of this invention are useful in improving or restoring β-cell function, so that they may be useful in treating type 1 diabetes or in delaying or preventing a patient with type 2 diabetes from needing insulin therapy.

The compounds, compositions, and medicaments as described herein are further useful for reducing the risks of adverse sequelae associated with metabolic syndrome, or Syndrome X, and in reducing the risk of developing atherosclerosis, delaying the onset of atherosclerosis, and/or reducing the risk of sequelae of atherosclerosis. Sequelae of atherosclerosis include angina, claudication, heart attack, stroke, and others.

The compounds may be useful for reducing appetite and body weight in obese subjects and may therefore be useful in reducing the risk of co-morbidities associated with obesity such as hypertension, atherosclerosis, diabetes, and dyslipidemia.

One aspect of the invention provides a method for the treatment and control of mixed or diabetic dyslipidemia, hypercholesterolemia, atherosclerosis, low HDL levels, high LDL levels, hyperlipidemia, and/or hypertriglyceridemia, which comprises administering to a patient in need of such treatment a therapeutically effective amount of a compound of the formulas described herein or a pharmaceutically acceptable salt thereof. The compound may be used alone or advantageously may be administered with a cholesterol biosynthesis inhibitor, particularly an HMG-CoA reductase inhibitor (e.g., simvastatin, atorvastatin, and the like). The compound may also be used advantageously in combination with other lipid lowering drugs such as cholesterol absorption inhibitors (e.g., stanol esters, sterol glycosides or azetidinones such as ezetimibe), ACAT inhibitors (e.g., avasimibe), CETP inhibitors (e.g. anacetrapib), niacin, bile acid sequestrants, microsomal triglyceride transport inhibitors, and bile acid reuptake inhibitors. Such combination treatments are useful for the treatment or control of conditions such hypercholesterolemia, atherosclerosis, hyperlipidemia, hypertriglyceridemia, dyslipidemia, high LDL, and low HDL.

Another aspect of the invention provides a method for the treatment and control of obesity or metabolic syndrome, which comprises administering to a patient in need of such treatment a therapeutically effective amount of a compound having the formulas described herein or a pharmaceutically acceptable salt thereof. The compound may be used alone or advantageously may be administered with an anti-obesity agent, such as a lipase inhibitor (e.g., orlistat,) or a monoamine neurotransmitter uptake inhibitor (e.g., sibutramine or phentermine). The compound may also be used advantageously in combination with CB-1 inverse agonists or antagonists (e.g., rimonabant or taranabant).

The present invention further relates to a method of treating hyperglycemia, diabetes or insulin resistance in a mammalian patient in need of such treatment which comprises administering to said patient a compound of the formulas described herein or a pharmaceutically acceptable salt thereof in an amount that is effective to treat hyperglycemia, diabetes or insulin resistance.

Yet another aspect of the invention that is of interest relates to a method of treating atherosclerosis in a mammalian patient in need of such treatment, comprising administering to said patient a compound of the formulas described herein or a pharmaceutically acceptable salt thereof in an amount that is effective to treat atherosclerosis.

Yet another aspect of the invention that is of interest relates to a method of delaying the onset of one of the aforementioned conditions and disorders where insulin resistance is a component in a mammalian patient in need thereof, comprising administering to the patient a compound of the formulas described herein or a pharmaceutically acceptable salt thereof in an amount that is effective to delay the onset of said condition.

Yet another aspect of the invention that is of interest relates to a method of reducing the risk of developing one of the aforementioned conditions and disorders where insulin resistance is a component in a mammalian patient in need thereof, comprising administering to the patient a compound of the formulas described herein or a pharmaceutically acceptable salt thereof in an amount that is effective to reduce the risk of developing said condition.

Yet another aspect of the invention that is of interest relates to a method of treating a condition or reducing the risk of developing a condition or delaying the onset of a condition selected from the group consisting of (1) hyperglycemia, (2) impaired glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) Syndrome X, (21) hypertension and other conditions and disorders where insulin resistance is a component, in a mammalian patient in need of such treatment, comprising administering to the patient a compound of the formulas described herein or a pharmaceutically acceptable salt thereof in an amount that is effective to treat said condition, and a compound selected from the group consisting of:
(a) DPP-IV inhibitors;
(b) insulin sensitizers selected from the group consisting of (i) PPAR agonists and (ii) biguanides;
(c) insulin and insulin mimetics;
(d) sulfonylureas and other insulin secretagogues;
(e) α-glucosidase inhibitors;
(f) glucagon receptor antagonists;
(g) GLP-1, GLP-1 mimetics, and GLP-1 receptor agonists;
(h) GIP,GIP mimetics, and GIP receptor agonists;
(i) PACAP, PACAP mimetics, and PACAP receptor 3 agonists;
(j) cholesterol lowering agents selected from the group consisting of
   (i) HMG-CoA reductase inhibitors, (ii) sequestrants, (iii) nicotinyl alcohol, nicotinic acid and salts thereof, (iv) PPARα agonists, (v) PPAR α/γ dual agonists, (vi) inhibitors of cholesterol absorption, (vii) acyl CoA:cholesterol acyltransferase inhibitors, and (viii) anti-oxidants;
(k) PPARδ agonists;
(l) antiobesity compounds;
(m) ileal bile acid transporter inhibitors;
(n) anti-inflammatory agents excluding glucocorticoids;
(o) protein tyrosine phosphatase-1B (PTP-1B) inhibitors; and
(p) antihypertensives including those acting on the angiotensin or renin systems, such as angiotensin converting enzyme inhibitors, angiotensin II receptor antagonists or renin inhibitors, (e.g., lisinopril, losartan); said compounds being administered to the patient in an amount that is effective to treat said condition.

For dosing purposes, any suitable route of administration may be employed for providing a mammal, especially a human, with an effective amount of a compound of the present invention. Dosage forms may include tablets, troches, dispersions, suspensions, solutions, capsules, creams, ointments, aerosols, and the like. Most preferably, compounds of the formulas described herein or a pharmaceutically acceptable salt thereof are administered orally. The effective dosage of active ingredient employed may vary depending on the particular compound employed, the mode of administration, the condition being treated and the severity of the condition being treated. Such dosage may be ascertained readily by a person skilled in the art.

When treating or controlling diabetes mellitus or other diseases for which compounds of the formulas described herein are indicated, generally satisfactory results are obtained when the compounds of the present invention are administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams. In the case of a 70 kg adult human, the total daily dose will generally be from about 1 milligram to about 350 milligrams. For a particularly potent compound, the dosage for an adult human may be as low as 0.1 mg. The dosage regimen may be adjusted within this range or even outside of this range to provide the optimal therapeutic response. Oral administration will usually be carried out using tablets or capsules. Examples of doses in tablets and capsules are 0.1 mg, 0.25 mg, 0.5 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 12 mg, 15 mg, 20 mg, 25 mg, 50 mg, 100 mg, 200 mg, 350 mg, 500 mg, 700 mg, 750 mg, 800 mg and 1000 mg. Other oral forms may also have the same or similar dosages.

Another aspect of the invention that is of interest is a pharmaceutical composition comprised of a compound of the formulas described herein or a pharmaceutically acceptable salt thereof in combination with a pharmaceutically acceptable carrier. The pharmaceutical compositions of the present invention comprise a compound of the formulas described herein or a pharmaceutically acceptable salt as an active ingredient, as well as a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids.

Salts of basic compounds encompassed within the term “pharmaceutically acceptable salt” refer to non-toxic salts of the compounds described herein which are generally prepared by reacting the free base with a suitable organic or inorganic acid. Representative salts of basic compounds described herein include, but are not limited to, the following: acetate, benzenesulfonate, benzoate, bicarbonate, bisulfate, bitartrate, borate, bromide, camsylate, carbonate, chloride, clavulanate, citrate, edetate, edisylate, estolate, esylate, formate, fumarate, gluceptate, gluconate, glutamate, hexylresorcinate, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, palmitate, pamoate (embonate), pantothenate, phosphate/diphosphate, polygalacturonate, salicylate, stearate, sulfate, subacetate, succinate, tannate, tartrate, teoclate, tosylate, triethiodide and valerate. Furthermore, where the compounds described herein carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, isopropylamine, lysine, methylglucamine, morpholine, piperazine, piperidine, polyamine resins, procaine, purines, theobromine, triethylamine, trimethylamine, tripropylamine, tromethamine, and the like.

A pharmaceutical composition may also comprise a prodrug, or a pharmaceutically acceptable salt thereof, if a prodrug is administered.

The compositions are typically suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the condition being treated and on the particular active ingredient selected. They may be conveniently presented in unit dosage form and prepared by any of the methods well-known in the art.

In practical use, compounds of the formulas described herein, or the pharmaceutically acceptable salts thereof can be combined as the active ingredient in intimate admixture with the pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage form. Solid pharmaceutical carriers are therefore typically employed. If desired, tablets may be coated by standard aqueous or nonaqueous techniques. Such compositions and preparations typically comprise at least about 0.1 percent of active compound, the remainder of the composition being the carrier. The percentage of active compound in these compositions may, of course, be varied and is conveniently between about 2 percent to about 60 percent of the weight of the dosage form. The amount of active compound in such therapeutically useful compositions is such that an effective dosage will be delivered.

Alternatively, the active compound can be administered intranasally as, for example, in the form of liquid drops or a spray.

The tablets, capsules and the like also typically contain a binder. Examples of suitable binders include gum tragacanth, acacia, gelatin and a synthetic or semisynthetic starch derivative, such as hydroxypropylmethylcellulose (HPMC); excipients such as dicalcium phosphate; a disintegrating agent such as corn starch, potato starch, alginic acid; a lubricant such as magnesium stearate; and in some instances, a sweetening agent such as sucrose, lactose or saccharin. When the dosage form employed is a capsule, it may contain, in addition to the components described above, a liquid carrier such as fatty oil.

Various other materials may be present as coatings or to modify the physical form of the dosage unit. For instance, tablets may be coated with shellac, sugar or both. Syrups and elixirs typically contain, in addition to the active ingredient, sucrose as a sweetening agent, methyl or propylparabens as a preservative, a dye and a flavoring such as cherry or orange flavor.

The compound of the formulas described herein or a pharmaceutically acceptable salt thereof may also be administered parenterally. Solutions or suspensions of these active compounds can be prepared in water, saline or another biocompatible vehicle, suitably mixed with a surfactant, buffer, and the like. Dispersions can also be prepared in glycerol, liquid polyethylene glycols and mixtures thereof in an oil. Under ordinary conditions of storage and use, these preparations can also contain a preservative to prevent the growth of microorganisms.

The pharmaceutical forms suitable for injectable use include sterile aqueous solutions and dispersions, and sterile powders for the extemporaneous preparation of sterile injectable solutions and dispersions. The preparation should be prepared under sterile conditions and be fluid to the extent that easy syringability exists. It should be sufficiently stable under the conditions of manufacture and storage and preserved against the growth of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and suitable oils.

As discussed supra, compounds of the present invention may be used in combination with other drugs that may also be useful in the treatment or amelioration of the individual diseases and conditions described herein. Such other drugs may be administered by a route and in an amount commonly used therefore, contemporaneously or sequentially with a compound of the formulas described herein or a pharmaceutically acceptable salt thereof. In the treatment of patients who have type 2 diabetes, insulin resistance, obesity, metabolic syndrome, neurological disorders, and co-morbidities that accompany these diseases, more than one drug is commonly administered. The compounds of this invention may generally be administered to a patient who is already taking one or more other drugs for these conditions.

When a compound of the formulas described herein is used contemporaneously with one or more other drugs, a pharmaceutical composition in unit dosage form containing such other drugs and the compound of the formulas described herein is preferred. However, the combination therapy also includes therapies in which a compound of the formulas described herein and one or more other drugs are administered on different overlapping schedules. It is also contemplated that when used in combination with one or more other active ingredients, the compound of the present invention and the other active ingredients may be used in lower doses than when each is used singly. Accordingly, the pharmaceutical compositions of the present invention include those that contain one or more other active ingredients, in addition to a compound of the formulas described herein.

Examples of other active ingredients that may be administered separately or in the same pharmaceutical composition in combination with a compound of the formulas described herein include, but are not limited to:

(1) dipeptidyl peptidase-IV (DPP-4) inhibitors (e.g., sitagliptin, alogliptin, linagliptin, vildagliptin, saxagliptin, teneligliptin, omarigliptin);

(2) insulin sensitizers, including (i) PPARγ agonists, such as the glitazones (e.g. pioglitazone, AMG 131, MBX2044, mitoglitazone, lobeglitazone, IDR-105, rosiglitazone, and balaglitazone), and other PPAR ligands, including (1) dual agonists (e.g., ZYH2, ZYH1, GFT505, chiglitazar, muraglitazar, □PPARα/γ aleglitazar, sodelglitazar, and naveglitazar); (2) PPARα agonists such as fenofibric acid derivatives (e.g., gemfibrozil, clofibrate, ciprofibrate, fenofibrate, bezafibrate), (3) selective PPARγ modulators (SPPARγM's), (e.g., such as those disclosed in WO 02/060388, WO 02/08188, WO 2004/019869, WO partial □2004/020409, WO 2004/020408, and WO 2004/066963); and (4) PPARγ agonists; (ii) biguanides, such as metformin and its pharmaceutically acceptable salts, in particular, metformin hydrochloride, and extended-release formulations thereof, such as Glumetza™, Fortamet™, and GlucophageXR™; and (iii) protein tyrosine phosphatase-1B (PTP-1B) inhibitors (e.g., ISIS-113715 and TTP814);

(3) insulin or insulin analogs (e.g., insulin detemir, insulin glulisine, insulin degludec, insulin glargine, insulin lispro, SBS 1000 and oral and inhalable formulations of insulin and insulin analogs);

(4) leptin and leptin derivatives and agonists;

(5) amylin and amylin analogs (e.g., pramlintide);

(6) sulfonylurea and non-sulfonylurea insulin secretagogues (e.g., tolbutamide, glyburide, glipizide, glimepiride, mitiglinide, meglitinides, nateglinide and repaglinide);

(7) α-glucosidase inhibitors (e.g., acarbose, voglibose and miglitol);

(8) glucagon receptor antagonists (e.g., NOXG15, LY2409021);

(9) incretin mimetics, such as GLP-1, GLP-1 analogs, derivatives, and mimetics; and GLP-1 receptor agonists (e.g., dulaglutide, semaglutide, albiglutide, exenatide, liraglutide, lixisenatide, taspoglutide, GSK2374697, ADX72231, RG7685, NN9924, ZYOG1, CJC-1131, and BIM-51077, including intranasal, transdermal, and once-weekly formulations thereof), and oxyntomodulin and oxyntomodulin analogs and derivatives;

(10) LDL cholesterol lowering agents such as (i) HMG-CoA reductase inhibitors (e.g., simvastatin, lovastatin, pravastatin, crivastatin, fluvastatin, atorvastatin, pitavastatin and rosuvastatin), (ii) bile acid sequestering agents (e.g., colestilan, colestimide, colesevalam hydrochloride, colestipol, cholestyramine, and dialkylaminoalkyl derivatives of a cross-linked dextran), (iii) inhibitors of cholesterol absorption, (e.g., ezetimibe), and (iv) acyl CoA:cholesterol acyltransferase inhibitors, (e.g., avasimibe);

(11) HDL-raising drugs, (e.g., niacin and nicotinic acid receptor agonists, and extended-release versions thereof; MK-524A, which is a combination of niacin extended-release and the DP-1 antagonist MK-524);

(12) antiobesity compounds;

(13) agents intended for use in inflammatory conditions, such as aspirin, non-steroidal anti-inflammatory drugs or NSAIDs, glucocorticoids, and selective cyclooxygenase-2 or COX-2 inhibitors;

(14) antihypertensive agents, such as ACE inhibitors (e.g., lisinopril, enalapril, ramipril, captopril, quinapril, and tandolapril), A-II receptor blockers (e.g., losartan, candesartan, irbesartan, olmesartan medoxomil, valsartan, telmisartan, and eprosartan), renin inhibitors (e.g., aliskiren), beta blockers, and calcium channel blockers;

(15) glucokinase activators (GKAs) (e.g., AZD6370);

(16) inhibitors of 11β-hydroxysteroid dehydrogenase type 1, (e.g., such as those disclosed in U.S. Pat. No. 6,730,690, and LY-2523199);

(17) CETP inhibitors (e.g., anacetrapib, evacetrapib and torcetrapib);

(18) inhibitors of fructose 1,6-bisphosphatase, (e.g., such as those disclosed in U.S. Pat. Nos. 6,054,587; 6,110,903; 6,284,748; 6,399,782; and 6,489,476);

(19) inhibitors of acetyl CoA carboxylase-1 or 2 (ACCT or ACC2);

(20) AMP-activated Protein Kinase (AMPK) activators, such as MB1055, ETC 1002;

(21) other agonists of the G-protein-coupled receptors: (i) GPR-109, (ii) GPR-119 (e.g., MBX2982, APD597, GSK1292263, HM47000, and PSN821), and (iii) GPR-40 (e.g., TAK875, CNX011, CNX 01162, CNX 01167, JTT 851, SARI, MR 1704, TUG 770, TUG 469, TUG499, ASP 4178);

(22) SSTR3 antagonists (e.g., such as those disclosed in WO 2009/001836);

(23) neuromedin U receptor agonists (e.g., such as those disclosed in WO 2009/042053, including, but not limited to, neuromedin S (NMS));

(24) SCD inhibitors;

(25) GPR-105 antagonists (e.g., such as those disclosed in WO 2009/000087);

(26) SGLT inhibitors (e.g., ASP1941, SGLT-3, empagliflozin, dapagliflozin, canagliflozin, BI-10773, PF-04971729, remogloflozin, TS-071, tofogliflozin, ipragliflozin, and LX-4211);

(27) inhibitors of acyl coenzyme A:diacylglycerol acyltransferase 1 and 2 (DGAT-1 and DGAT-2);

(28) inhibitors of fatty acid synthase;

(29) inhibitors of acyl coenzyme A:monoacylglycerol acyltransferase 1 and 2 (MGAT-1 and MGAT-2);

(30) agonists of the TGR5 receptor (also known as GPBAR1, BG37, GPCR19, GPR131, and M-BAR);

(31) ileal bile acid transporter inhibitors;

(32) PACAP, PACAP mimetics, and PACAP receptor 3 agonists;

(33) PPAR agonists;

(34) protein tyrosine phosphatase-1B (PTP-1B) inhibitors;

(35) IL-1b antibodies, (e.g., XOMA052 and canakinumab);

(36) bromocriptine mesylate and rapid-release formulations thereof; or

(37) GPR 120 agonists (such as KDT501).

Another aspect of the invention that is of interest relates to the use of a compound of the formulas described herein or a pharmaceutically acceptable salt thereof in the manufacture of a medicament for use in treating a disease or condition described herein.

The compounds of the invention can be prepared using the synthetic schemes described herein as well as any of several alternate methods which will be apparent to a chemist skilled in the art. The following abbreviations may be used in the synthetic schemes or Examples: BuTMDOB is trans 2-butyl-N,N,N,N-tetramethyl-1,3,2-dioxaborolane-4,5-dicarboxamide, as specified R,R or S,S; CBz is carbobenzyloxy; CPME is cyclopentyl methyl ether; DCM is dichloromethane; DMAP is dimethylaminopyridine; DMF is N,N-dimethylformamide; DMSO is dimethyl sulfoxide; EDC is 1-ethyl-3-[3-(dimethylamino)propyl]-carbodiimide HCl; EtOAc is ethyl acetate; EtOH is ethanol; HCl is hydrochloric acid; HOBt is 1-hydroxybenzotriazole; HPLC is high performance liquid chromatography; iPrOAc is isopropyl acetate; KHMDS is potassium hexamethyldisilazane; LRMS is low resolution mass spectrometry; M is molar; mmol is millimole; NaHMDS is sodium hexamethyldisilazane; n-BuLi is n-butyllithium; RTis RT; OTf is triflate; PPh3 is triphenylphosphine; TEA is triethylamine; TFA is trifluoroacetic acid; THF is tetrahydrofuran; TLC is thin layer chromatography; TPAP is tetrapropylammonium perruthenate.

Reaction Schemes below illustrate in exemplary fashion the methods employed in the synthesis of the compounds of the present invention of Formula I; other routes may be contemplated as well.

### General Schemes

Substituted aryl and heteroaryl coupling intermediates shown in the schemes are commercially available or may be prepared from readily accessible aryl, heterocyclic, or other congeners via a host of routes. Many intermediates are accessible through either modification of a pre-formed heteroaryl scaffold or through de novo ring synthesis.

The cyclopropyl residue in the connecting chain of the present examples may be introduced by any of several methods. A particularly convenient method is outlined in Scheme 1 below. Conversion of the readily available piperidine aldehyde to the trans olefins, the cyclopropanation precursor, was achieved by a two step sequence including Horner-Emmons olefinaton and reduction. Charette's Et₂Zn/CH₂I₂ cyclopropanation yields racemic, diastereomerically or enantiomerically enriched cyclopropyl analogs. In the absence of an auxiliary chiral Lewis acid the trans allylic olefins yield good yields of the desired racemic analogs.

With the addition of the auxiliary chiral Lewis acid RR or SS BuTMDOB, the same cyclopropanation protocol leads to very good ratios of the desired enantiomer.

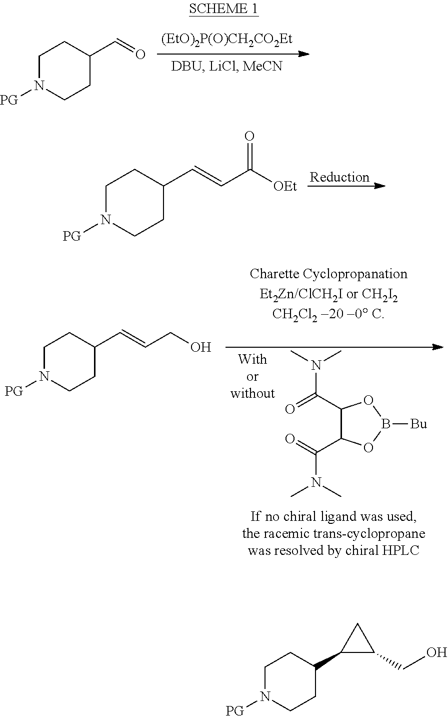

The trans cyclopropane methanols prepared in Scheme 1 can be further homolagated to the corresponding cyclopropane ethanols through a three step sequence, as outlined in Scheme 2.

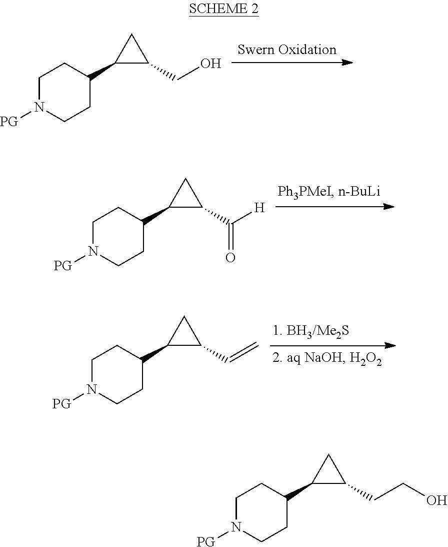

Further more, the trans cyclopropane ethanols can be converted to the corresponding amines, as outlined in Scheme 3.

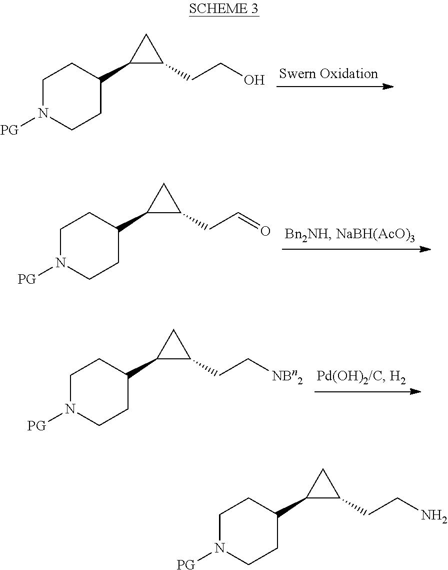

Using the alcohols prepared as outlined in Scheme 1, many analogs may be prepared by different routes. Scheme 4 outlines a particularly convenient method for conversion of the cyclopropyl alcohol to substituted aryl/heteroaryl alkyl ethers via treatment with aryl/heteroaryl alkyl halides in the presence of a base, such as NaHMDS or KHMDS usually heated to 70° C., for a period of 2 to 24 hours. Depending on the amino protecting group, several methods can be used for removal which will be apparent to a chemist skilled in the art. For example, most commonly used t-butylcarbonyl can be removed via treatment with an acid, e.g., HCl or TFA. Another commonly used protecting group is CBz which can be removed via hydrogenation.

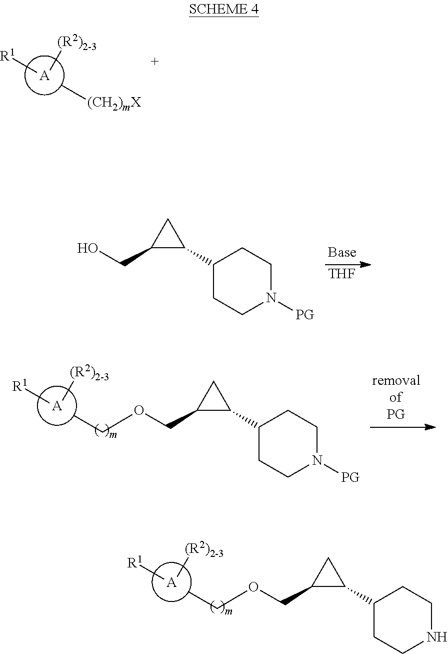

Using the alcohols prepared as outlined in Schemes 2, many analogs may be prepared following the sequence in Scheme 5.

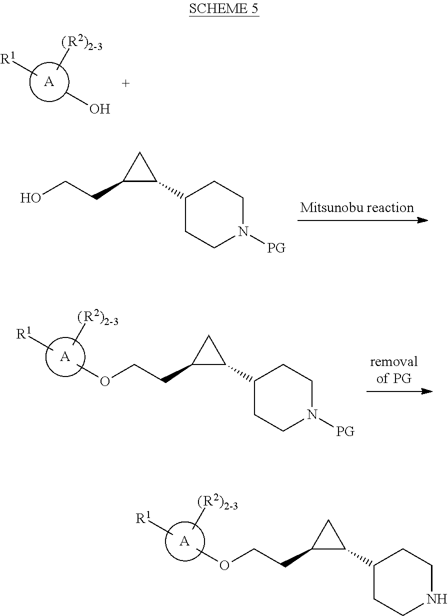

The trans cyclopropyl alcohol can be converted to a leaving group such as a tosylate or iodide via treatment with tosyl chloride in the presence of an organic base, such as TEA, and an activating agent, such as DMAP, in the appropriate solvent, or by treatment with iodine and triphenylphosphine in the presence of imidazole. This tosylate/iodide can then be treated with the choice of aryl/heteroaryl alkyl alcohols in the presence of base, such as sodium hydride to form the desired aryl/heteroaryl alkyl ethers, as illustrated in Scheme 6.

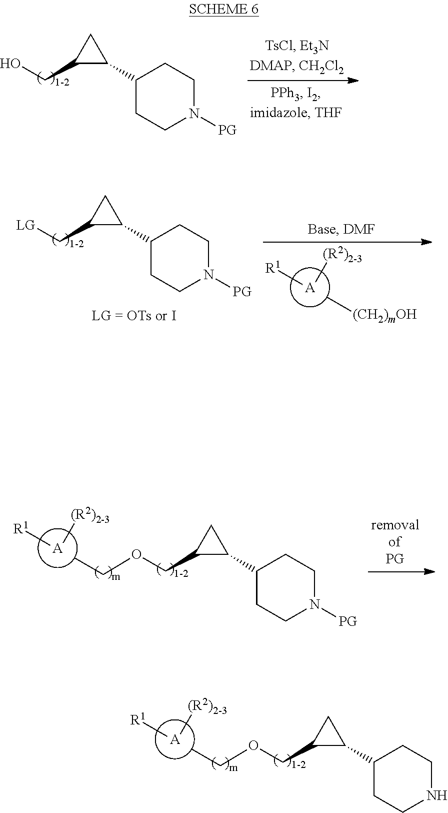

Introduction of the piperidine nitrogen substituent can be accomplished by a particularly wide variety of routes. Some of the most versatile routes for the examples reported here are represented in the following schemes. The formation of carbamate analogs are outlined in Scheme 7. Commercially available alkyl or aryl chloroformates or preformed succinimides can be used in the acylation of the nitrogen of the cyclopropyl intermediate with a base such as DIEA or TEA, to yield the carbamate GPR119 agonist analogs. This procedure is particularly useful for targeting several different carbamate analogs of a particularly interesting aryl or heteroarylalkyl ether.

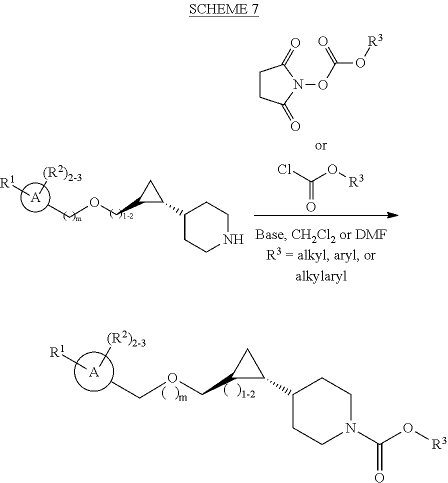

Introduction of the piperidine nitrogen 5-membered heterocyclic substituents can be accomplished by a number of routes. One of the most versatile routes for the examples reported here are represented in Scheme 8. The amine of the piperidine is converted to a cyano substituted piperidine by treatment with cyanogen bromide in the presence of base in a suitable chlorinated solvent at temperatures from 0° C. to reflux. The cyano intermediate can then be converted to a 3-substituted 1,2,4-oxadiazole by zinc chloride mediatyed reaction with an N-hydroxyalkylimidamide or N-hydroxyarylimidamide, followed by acid mediated cyclization.

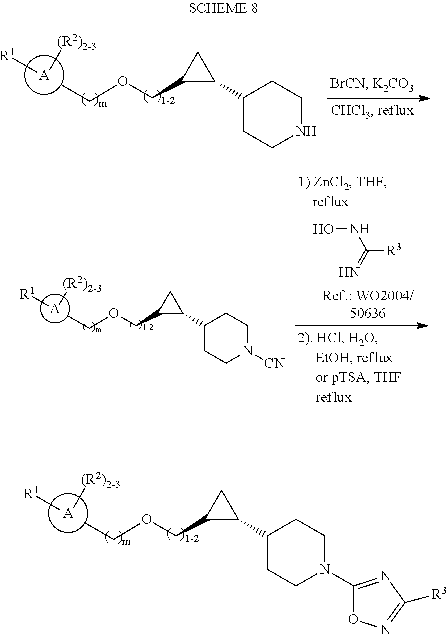

Direct displacement of labile heteroaryl halides or other leaving groups can often be used to introduce the nitrogen substituent directly as shown in Scheme 9.

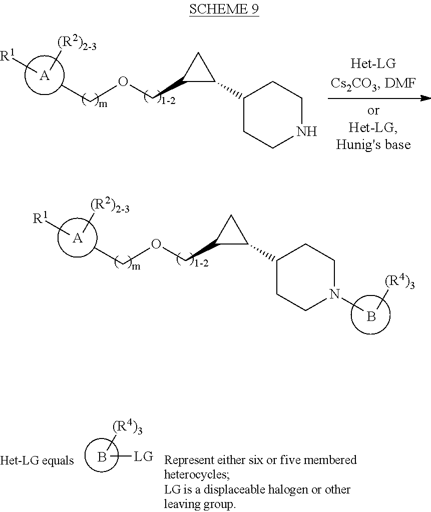

The amino analogs can be obtained from the trans cyclopropane ethyl amines, prepared as outlined in Scheme 3, through an S_{N}Ar reaction or palladium mediated C—N bond formation (Scheme 10).

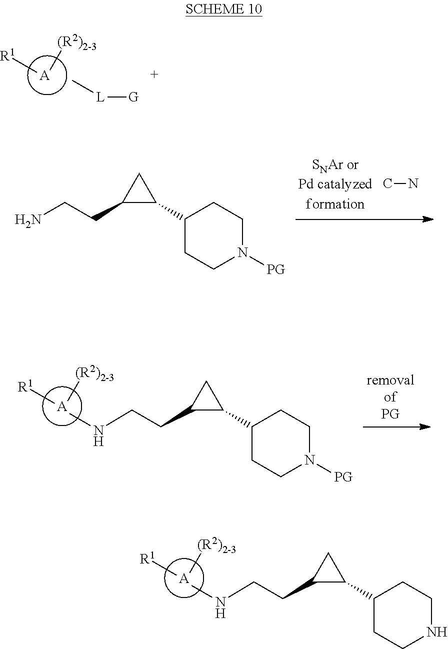

The order of introduction of piperidine N substituents and aryl/heteroarylalkyl ether is easily inverted by removal of piperidine protecting group first. Following derivatisation of the piperidine nitrogen using the methods outlined in the above schemes, further elaboration of the primary alcohol may be achieved by base mediated reaction with aryl/heteroarylalkyl halide, or activation of the alcohol to a leaving group and displacement with aryl/heteroarylalkyl alcohol as outlined in scheme 11.

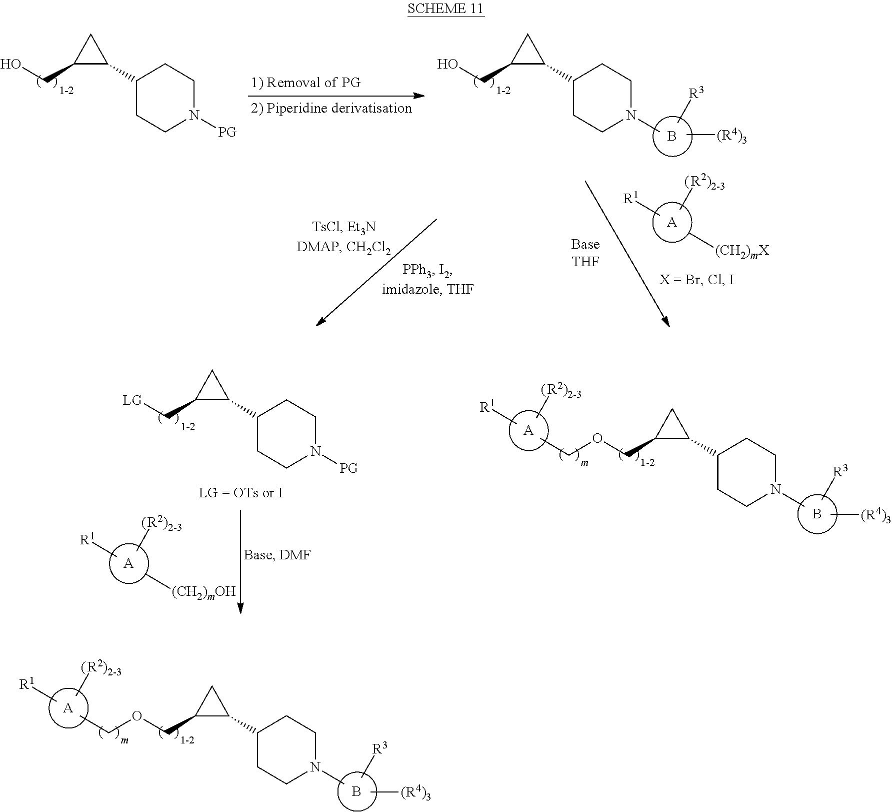

### INTERMEDIATES

### Intermediate 1

### Benzyl 4-((1R,2S)-2-(hydroxymethyl)cyclopropyl)piperidine-1-carboxylate

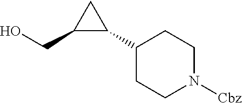 [SMILES: O=C(OCc1ccccc1)N1CCC([C@H]2C[C@@H]2CO)CC1]

### Step A: (E)-Benzyl 4-(3-ethoxy-3-oxoprop-1-enyl)piperidine-1-carboxylate

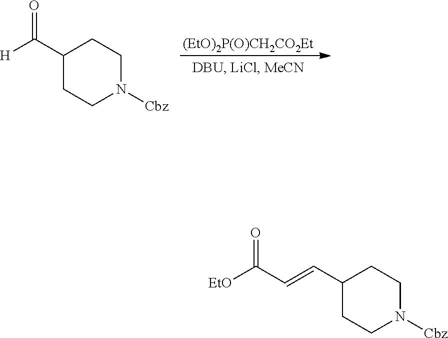 [SMILES: "(EtO)2P(O)CH2CO2Et" ; O=CC1CCN(C(=O)OCc2ccccc2)CC1 ; CCOC(=O)/C=C/C1CCN(C(=O)OCc2ccccc2)CC1]

To a solution of ethyl diethylphosphonoacetate (125.5 g, 0.56 mol) and LiCl (27 g, 0.64 mol) in CH₃CN (1.5 L) was added DBU (85 g, 0.56 mol), followed by the aldehyde (131 g, 0.53 mol) in CH₃CN (200 mL). The mixture was stirred at room temperature for 16 h. The reaction solution was filtered. The filtrate was concentrated, dissolved in water and extracted with EtOAc. The organic layer was concentrated, the residue was purified by column chromatography (PE: EtOAc=10:1˜5:1) to give the product as a yellow oil (140 g, yield: 83%). ESI m/e (M+H⁺): 318.2.

### Step B: (E)-Benzyl 4-(3-hydroxyprop-1-enyl)piperidine-1-carboxylate

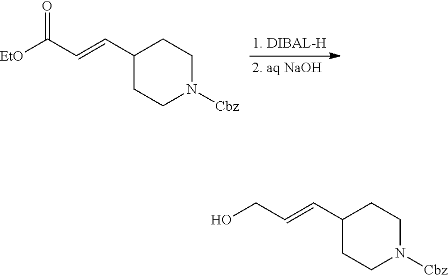 [SMILES: CCOC(=O)/C=C/C1CCN(C(=O)OCc2ccccc2)CC1 ; O=C(OCc1ccccc1)N1CCC(/C=C/CO)CC1]

To a solution of Step A product (200 g, 0.62 mol) in DCM (3 L) was added dropwise DIBAL-H (1.56 L, 1.56 mol) at 0° C. under N₂. The mixture was stirred at 0° C. for 5 h. Water (62 mL) was added dropwise, followed by a slow addition of aqueous NaOH solution (15%, W/W, 62 mL), and water (156 mL). The mixture was stirred for 40 min, dried over MgSO₄ and filtered. The filtrate was concentrated, and the residue was purified by column chromatography (PE:EtOAc=5:1˜2:1) to give the product as a yellow oil (55 g, yield: 32%). ESI m/e (M+H⁺): 276.4.

### Step C: Benzyl 4-((1R,2S)-2-(hydroxymethyl)cyclopropyl)piperidine-1-carboxylate

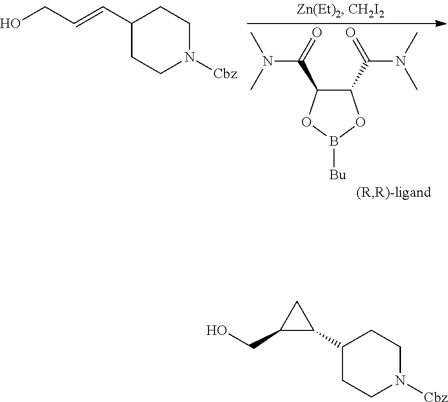

Diethylzinc (10.24 ml, 100 mmol) was added to a solution of anhydrous CH₂Cl₂ (66.6 ml) and anhydrous DME (10.38 ml, 100 mmol) that had been degassed and cooled to −10° C. Diiodomethane (16.11 ml, 200 mmol) was slowly added to this mixture and the resulting solution was stirred at −10° C. for 20 min. A solution of Step B product (5.500 g, 19.98 mmol) and (R, R) dioxaborolane ligand (6.48 g, 23.97 mmol) in anhydrous DCM (3 mL) that had been degassed and cooled to −10° C. was then added to the diethylzinc mixture over the course of 5 min. The reaction was stirred for 5 h at 0° C. Then the reaction was quenched at 0° C. by the slow addition of a small volume of sat. NH₄Cl solution, followed by a volume equal to the amount of organic solvent. 6 mL of AcOH was added to this mixture and the layers cut in a separaton funnel. The aqueous phase was extracted with DCM, and the combined organic phases were stirred with an equal volume of 4N KOH solution overnight. The subsequent morning the phases were separated and the organic layer extracted with sat. NH₄Cl. The organic phase was then dried over MgSO₄, filtered and concentrated under reduced pressure to afford a yellow tinged oil. Further purification by silica column (100 g SNAP column, Biotage system) eluting with a range of 30-60% EtOAc/Hex over 11CV afforded 3.6 g (12.44 mmol, 62.3% yield) of desired product as a clear, colorless oil. [α]_{D}=+12. ¹H NMR (400 MHz, CDCl₃) δ 7.37 (m, 5H), 5.13 (s 2H), 4.17 (m, 2H), 3.42 (d, 2H), 2.72 (m, 2H), 1.75 (m, 2H), 1.39-1.20 (m, 3H), 0.92 (m, 1H), 0.76 (m, 1H), 0.45 (m, 1H), 0.38 (m, 2H). ESI m/e (M+H⁺): 290.2.

### Intermediate 2

### Benzyl 4-((1S,2R)-2-(hydroxymethyl)cyclopropyl)piperidine-1-carboxylate

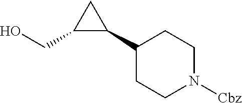 [SMILES: O=C(OCc1ccccc1)N1CCC([C@@H]2C[C@H]2CO)CC1]

Intermediate 2 was prepared in the same way as Intermediate 1 except for Step C where (S,S) dioxaborolane ligand was used.

### Intermediate 3

### tert-Butyl 4-((1R,2S)-2-(hydroxymethyl)cyclopropyl)piperidine-1-carboxylate

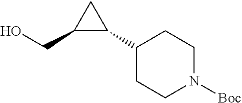 [SMILES: CC(C)(C)OC(=O)N1CCC([C@H]2C[C@@H]2CO)CC1]

A mixture of Intermediate 1(5.0 g, 17.3 mmol), Boc₂O (3.8 mg, 17.3 mmol) and 10% palladium on carbon (20%, w/w, 1.0 g) in ethyl acetate (100 mL) was hydrogenated under 30 psi of Hydrogen at 30° C. Then the mixture was cooled to room temperature and the catalyst was filtered off. The filtrate was concentrated in vacuo to give the title compound as colorless oil (4.2 g, yield: 95%). MS ESI [M+H]⁺ 256.

### Intermediate 4

### Benzyl 4-((1R,2R)-2-(2-hydroxyethyl)cyclopropyl)piperidine-1-carboxylate

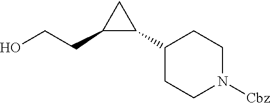 [SMILES: O=C(OCc1ccccc1)N1CCC([C@H]2C[C@@H]2CCO)CC1]

### Step A: Benzyl 4-((1R,2S)-2-formylcyclopropyl)piperidine-1-carboxylate

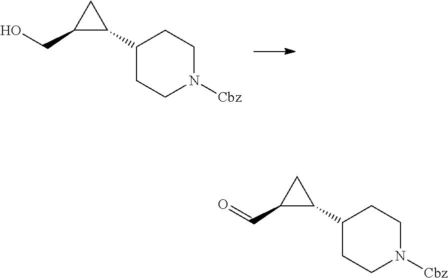 [SMILES: O=C(OCc1ccccc1)N1CCC([C@H]2C[C@@H]2CO)CC1 ; O=C[C@H]1C[C@@H]1C1CCN(C(=O)OCc2ccccc2)CC1]

At −78° C., to a mixture of (COCl)₂ (23 g, 180 mmol) in dry DCM (500 mL) was added DMSO (28 g, 359 mmol), then the mixture was stirred at −78° C. for 30 min. Intermediate 1 (26 g, 90 mmol) was added, and the reaction mixture was stirred at −78° C. for 1 h. Et₃N (73 g, 719 mmol) was added, then the reaction mixture was stirred at −30° C. for 1 h. The mixture was washed with brine, dried over Na₂SO₄ and concentrated to give the aldehyde as colorless oil (27 g, crude). ESI m/e (M+H): 288.4.

### Step B: Benzyl 4-((1R,2R)-2-vinylcyclopropyl)piperidine-1-carboxylate

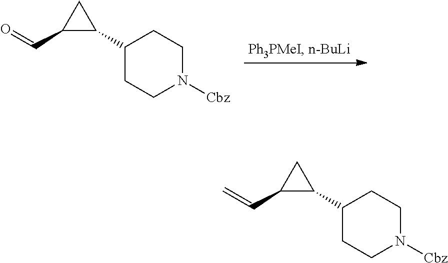 [SMILES: O=C[C@H]1C[C@@H]1C1CCN(C(=O)OCc2ccccc2)CC1 ; C=C[C@H]1C[C@@H]1C1CCN(C(=O)OCc2ccccc2)CC1]

At −78° C., to a mixture of Ph₃PMeI (42 g, 104 mmol) in dry THF (500 mL) was added n-BuLi (40 mL, 100 mmol), then the mixture was stirred at −78° C. for 30 min. Step A product (26 g, crude) was added, and the reaction mixture was stirred at room temperature for 1 h. After the reaction was quenched by saturated aquous ammonium chloride solution, the mixture was extracted with ethyl acetate (300 mL×3), and the combined organic portions were washed with brine, dried over Na₂SO₄ and concentrated to give crude product. Further purification by column chromatography (PE: EtOAc=20:1˜5:1) afforded the product as a colorless oil (21 g, yield: 82% over two steps). ESI m/e (M+H): 286.2.

### Step C: Benzyl 4-((1R,2R)-2-(2-hydroxyethyl)cyclopropyl)piperidine-1-carboxylate

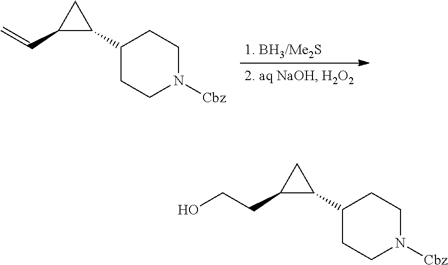 [SMILES: C=C[C@H]1C[C@@H]1C1CCN(C(=O)OCc2ccccc2)CC1 ; O=C(OCc1ccccc1)N1CCC([C@H]2C[C@@H]2CCO)CC1]

At 0° C., to a solution of Step B product (20 g, 70 mmol) in dry THF (500 mL) was added BH₃/Me₂S (3.5 mL, 35 mmol). The reaction mixture was stirred at room temperature for 2 h. Aqueous sodium hydroxide solution (5 M, 100 mL) and Hydrogen peroxide solution (30%, 100 mL) were added very slowly, then the mixture was stirred at room temperature for 1 h. The reaction mixture was extracted with ethyl acetate (200 mL×3), and the combined organic portions were washed with brine, dried over Na₂SO₄ and concentrated to give crude product. Purification by column chromatography (PE: EtOAc=10:1˜3:1) afforded the desired product as a colorless oil (18 g, yield: 85%). ¹H NMR (400 MHz, CDCl₃) δ 7.1 (m, 5H), 4.85 (s, 2H), 3.90 (m, 2H), 3.42 (t, 2H), 2.45 (m, 2H), 1.47 (m, 2H), 1.30 (m, 1H), 1.15 (m, 1H), 0.98 (m, 2H), 0.48 (m, 1H), 0.30 (m, 1H), 0.10 to -0.03 (m, 3H). ESI m/e (M+H+): 304.3.

### Intermediate 5

### Benzyl 4-((1S,2S)-2-(2-hydroxyethyl)cyclopropyl)piperidine-1-carboxylate

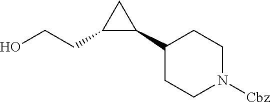 [SMILES: O=C(OCc1ccccc1)N1CCC([C@@H]2C[C@H]2CCO)CC1]

Starting from Intermediate 2, this compound was prepared in the same way as Intermediate 4. ¹H NMR (400 MHz, CDCl₃) δ 7.1 (m, 5H), 4.85 (s, 2H), 3.90 (m, 2H), 3.42 (t, 2H), 2.45 (m, 2H), 1.47 (m, 2H), 1.30 (m, 1H), 1.15 (m, 1H), 0.98 (m, 2H), 0.48 (m, 1H), 0.30 (m, 1H), 0.10 to −0.03 (m, 3H). ESI m/e (M+H⁺): 304.3.

### Intermediate 6

### tert-Butyl 4-((1R,2R)-2-(2-hydroxyethyl)cyclopropyl)piperidine-1-carboxylate

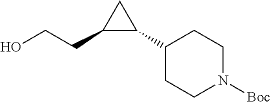 [SMILES: CC(C)(C)OC(=O)N1CCC([C@H]2C[C@@H]2CCO)CC1]

### Step A: tert-Butyl 4-((1R,2R)-2-(2-hydroxyethyl)cyclopropyl)piperidine-1-carboxylate

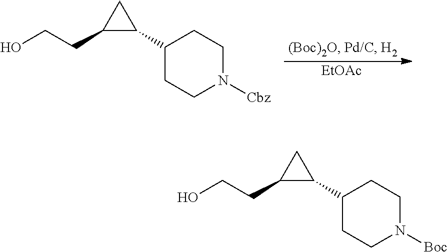 [SMILES: "EtOAc" ; O=C(OCc1ccccc1)N1CCC([C@H]2C[C@@H]2CCO)CC1 ; CC(C)(C)OC(=O)N1CCC([C@H]2C[C@@H]2CCO)CC1]

A mixture of Intermediate 4 (2.0 g, 6.6 mmol), (Boc)₂O (1.5 g, 7.2 mmol) and 10% palladium on carbon (20%, w/w, 0.4 g) in EtOAc (100 mL) was hydrogenated under 30 psi of hydrogen at 30° C. Then the reaction was cooled to room temperature and the catalyst was filtered off. The filtrate was concentrated in vacuo to give the desired product as a colorless oil (2.0 g). ESI m/e (M+H⁺): 270.2.

### Intermediate 7

### tert-Butyl 4-((1S,2R)-2-(2-aminoethyl)cyclopropyl)piperidine-1-carboxylate

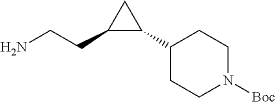 [SMILES: CC(C)(C)OC(=O)N1CCC([C@H]2C[C@@H]2CCN)CC1]

### Step A: tert-Butyl 4-((1R,2R)-2-(2-oxoethyl)cyclopropyl)piperidine-1-carboxylate

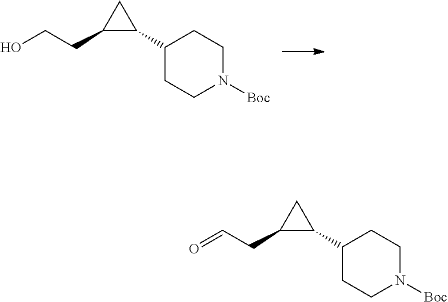 [SMILES: CC(C)(C)OC(=O)N1CCC([C@H]2C[C@@H]2CCO)CC1 ; CC(C)(C)OC(=O)N1CCC([C@H]2C[C@@H]2CC=O)CC1]

To a stirred solution of Oxalyl dichloride (1.4 g, 11 mmol) in DCM (30 mL) was added dropwise a solution of DMSO (1.4 g, 18 mmol) in DCM (5 mL) at −78° C., and the resulted mixture was stirred at this temperature for 30 min. Then Intermediate 6 (0.74 mmol, 2.0 g) in DCM (5 mL) was added dropwise, and the reaction mixture was stirred for another 1 h at this temperature. TEA (2.0 g, 20 mmol) was added and the mixture was stirred for 30 min at room temperature. Then the mixture was quenched with water (50 mL), extracted with DCM (30 mL x 3), and the combined organic portions were washed with brine, dried over Na₂SO₄ and concentrated to give the aldehyde as a colorless oil (2.0 g, crude). (ESI) m/e (M+H⁺): 268.2.

### Step B: tert-Butyl 4-((1S,2R)-2-(2-(dibenzylamino)ethyl)cyclopropyl)piperidine-1-carboxylate

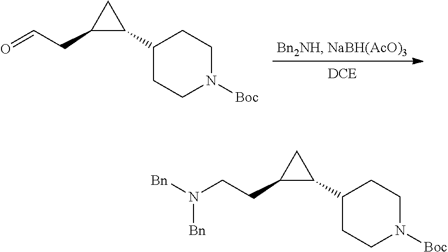 [SMILES: CC(C)(C)OC(=O)N1CCC([C@H]2C[C@@H]2CC=O)CC1 ; CC(C)(C)OC(=O)N1CCC([C@H]2C[C@@H]2CCN(Cc2ccccc2)Cc2ccccc2)CC1]

A solution of the aldehyde prepared in Step A (2.0 g, 7.5 mmol) and dibenzyl-amine (1.6 g, 8.2 mmol) in DCE (10 mL) was stirred at room temperature for 3 h. NaBH(AcO)₃ (3.2 g, 15 mmol) was added, and the mixture was stirred at room temperature for 2 h. The reaction solution was diluted with water and extracted with DCM. The organic layer was concentrated, and the residue was purified by column chromatography (PE: EtOAc=20:1˜10:1) to give the compound product as a colorless oil (2.0 g, yield: 59%). ESI m/e (M+H⁺): 449.3.

### Step C: tert-Butyl 4-((1S,2R)-2-(2-aminoethyl)cyclopropyl)piperidine-1-carboxylate

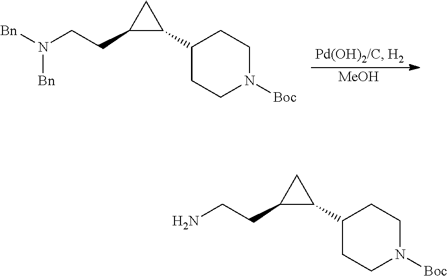 [SMILES: "MeOH" ; CC(C)(C)OC(=O)N1CCC([C@H]2C[C@@H]2CCN(Cc2ccccc2)Cc2ccccc2)CC1 ; CC(C)(C)OC(=O)N1CCC([C@H]2C[C@@H]2CCN)CC1]

A mixture of Step B product (2.0 g, 4.5 mmol) and 10% palladium hydroxide on carbon (20%, w/w, 0.4 g) in MeOH (100 mL) was hydrogenated under 30 psi of hydrogen at 30° C. overnight. Then the mixture was cooled to room temperature and the catalyst was filtered off. The filtrate was concentrated in vacuo to give the desired product as a colorless oil (1.0 g, yield 83%). ESI m/e (M+H⁺): 269.4.

### Intermediate 8

### tert-Butyl 4-((1R,2S)-2-(2-aminoethyl)cyclopropyl)piperidine-1-carboxylate

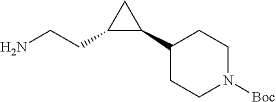 [SMILES: CC(C)(C)OC(=O)N1CCC([C@@H]2C[C@H]2CCN)CC1]

Similarly, Intermediate 8 was prepared as described as above using the other enantiomer. ESI m/e (M+H⁺): 269.4.

### Intermediate 9

### Preparation of 2-chloro-5-methoxymethylpyrimidine

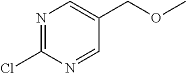

To a solution of 2-chloro-5-hydroxymethyl-pyrimidine (9.0 g, 62 mmol) in 70 ml of anhydrous DMF was added methyl iodide (6 eq. 370 mmol, 23 ml). The mixture was cooled to 0° C., then NaH (2.61 g, 1.05 eq.) was added in portions over 5 mins. The resulting mixture was stirred 25 min. at 0° C., then 25 min. at rt. The reaction mixture was then cooled in ice bath, and quenched by addition of saturated NH₄Cl aq. solution (200 ml), extracted with ether (150 ml×3). The combined organic layers were washed by brine, dried over Na₂SO₄, filtered and concentrated in vacuo. The residue was purified by ISCO column (330 g of silica gel) using ethyl acetate in hexane (0-90% ethyl acetate, 2500 ml, then 1000 ml of ethyl acetate) to give 6.5 g (66%) of the title compound: ¹H NMR (500 MHz, CDCl₃) δ 8.60 (s, 2H), 4.48 (s, 2H), 3.45 (s, 3H). MS ESI m/z 159.2 (M+H).

### Intermediate 10

### 2-((1R,2R)-2-(1-(5-(Methoxymethyl)pyrimidin-2-yl)piperidin-4-yl)cyclopropyl)ethyl 4-methylbenzenesulfonate

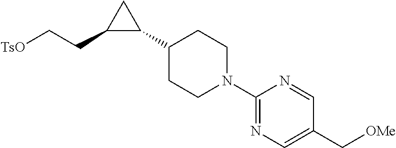 [SMILES: COCc1cnc(N2CCC([C@H]3C[C@@H]3CCOS(=O)(=O)c3ccc(C)cc3)CC2)nc1]

### Step A: 2-((1R,2R)-2-(Piperidin-4-yl)cyclopropyl)ethanol

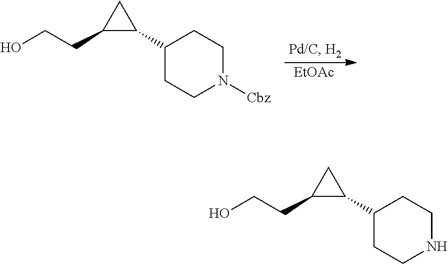 [SMILES: "EtOAc" ; O=C(OCc1ccccc1)N1CCC([C@H]2C[C@@H]2CCO)CC1 ; OCC[C@H]1C[C@@H]1C1CCNCC1]

A mixture of Intermediate 4 (2.0 g, 6.6 mmol) and 10% palladium on carbon (20%, w/w, 0.4 g) in EtOAc (100 mL) was hydrogenated under 30 psi of hydrogen at 30° C. Then the reaction was cooled to room temperature and the catalyst was filtered off. The filtrate was concentrated in vacuo to give the desired product that was used in the next step without further purification.

### Step B: 2-((1R,2R)-2-(1-(5-(Methoxymethyl)pyrimidin-2-yl)piperidin-4-yl)cyclopropyl)ethanol

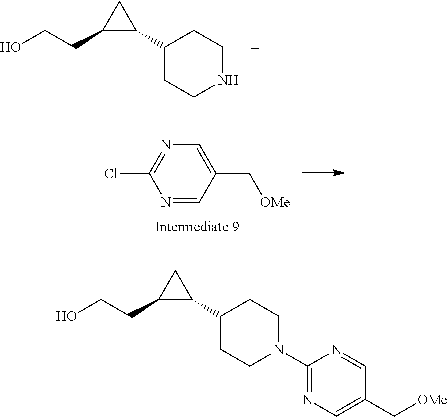 [SMILES: OCC[C@H]1C[C@@H]1C1CCNCC1 ; COCc1cnc(Cl)nc1 ; COCc1cnc(N2CCC([C@H]3C[C@@H]3CCO)CC2)nc1]

To a stirred solution of Intermediate 9 (1.5 g, 9.49 mmol) in DMF (15 mL) was added Step A product (1.77 g, 10.44 mmol), Cs₂CO₃ (4.64 g, 10.24 mmol). The reaction mixture was stirred at ambient temperature for 4 h. TLC showed that the reaction was complete. The reaction was diluted with EtOAc, washed with water (3×) and brine. The organic layer was dried over MgSO₄, filtered and concentrated to dryness. Purification of the residue by column chromatography (hexanes/EtOAc=1:1) afforded the product as a colorless oil (600 mg, 22%). MS ESI [M+H]+ 292. found 292.

### Step C: 2-((1R,2R)-2-(1-(5-(Methoxymethyl)pyrimidin-2-yl)piperidin-4-yl)cyclopropyl)ethyl 4-methylbenzenesulfonate

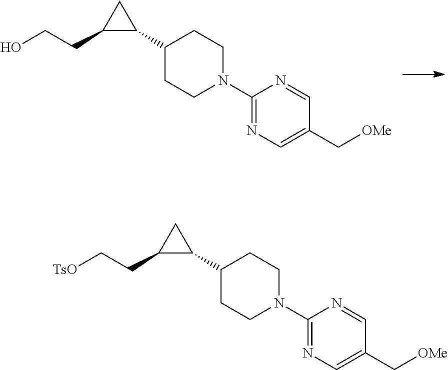 [SMILES: COCc1cnc(N2CCC([C@H]3C[C@@H]3CCO)CC2)nc1 ; COCc1cnc(N2CCC([C@H]3C[C@@H]3CCOS(=O)(=O)c3ccc(C)cc3)CC2)nc1]

To a stirred solution of Step B product (0.6 g, 2.06 mmol) in DCM (15 mL) was added TsCl (666 mg, 3.51 mmol), TEA (625 mg, 6.19 mmol), and DMAP (40 mg, 0.33 mmol). The reaction mixture was stirred at ambient temperature for 2 h. TLC showed the reaction was complete. The reaction mixture was directly loaded on silica column and eluted with hexanes/EtOAc (3:1 v/v) to gave the desired product as a colorless oil (360 mg, 39%). MS ESI [M+H]+ 446.

### Intermediate 11

### Cyclopropylmethyl 2,5-dioxopyrrolidin-1-yl carbonate

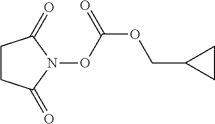 [SMILES: O=C(OCC1CC1)ON1C(=O)CCC1=O]

### Step A: Cyclopropylmethyl 2,5-dioxopyrrolidin-1-yl carbonate

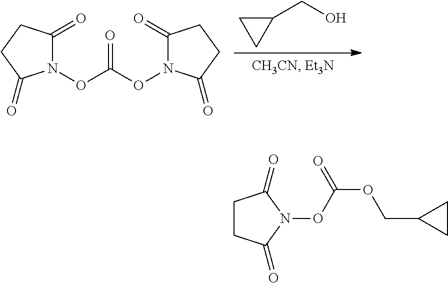

To a stirred solution of cyclopropylmethanol (1.0 g, 13.8 mmol) in acetonitrile (15 mL) was added bis(2,5-dioxopyrrolidin-1-yl) carbonate (7.1 g, 27.7 mmol) and triethylamine (5.8 mL, 41.6 mmol). The reaction was stirred overnight, then quenched with saturated NaHCO3 solution (aq.) and extracted with ethyl acetate (3×50 mL), dried over sodium sulfate, filtered and concentrated under reduced pressure to provide the title compound (3.2 g, quant.) as an amber oil: ¹H NMR (300 MHz, CDCl₃) δ 4.16 (d, J=7.5 Hz, 2H), 2.84 (s, 4H), 1.26 (m, 1H), 0.70-0.64 (m, 2H), 0.40-0.35 (m, 2H).

### Intermediate 12

### 2,5-Dioxopyrrolidin-1-yl 1-methylcyclopropyl carbonate

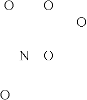

The title compound was synthesized according to the method described above for Intermediate 11 using the appropriate alcohol.

### Intermediate 13

### Cyclobutyl 2,5-dioxopyrrolidin-1-yl carbonate

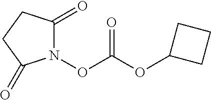 [SMILES: O=C(OC1CCC1)ON1C(=O)CCC1=O]

The title compound was synthesized according to the method described above for Intermediate 11 using the appropriate alcohol.

### Intermediate 14

### 5-Bromo-2-(bromomethyl)pyridine

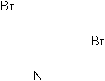

### Step A: 5-bromo-2-(bromomethyl)pyridine

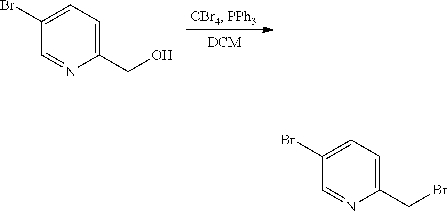 [SMILES: OCc1ccc(Br)cn1 ; BrCc1ccc(Br)cn1]

5-Bromo-2-hydroxymethylpyridine (1 g, 5.32 mmol) was dissolved in dichloromethane (26.6 ml) and cooled to 0° C. Triphenylphosphine (1.604 g, 6.12 mmol) was added followed by carbon tetrabromide (2.028 g, 6.12 mmol) which caused the reaction to become yellow and heterogeneous. After 48 h, the mixture was concentrated by half and directly purified by silica gel column chromatography (0-37%, EtOAc-hexanes) to yield the title compound. ¹H NMR (500 MHz, CDCl₃) δ 8.64 (t, J 2.7 Hz, 1H), 7.82 (m, 1H), 7.35 (dd, J 7.8 & 3.1 Hz, 1H), 4.51 (s, 2H).

### Intermediate 15

### 3-Fluoro-4-bromobenzyl bromide

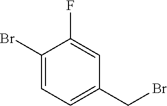

### Step A: (4-Bromo-3-fluorophenyl)methanol

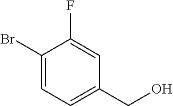

A 0° C. solution of 4-bromo-3-fluoro benzoic acid (2.3 g, 10.50 mmol) in dry THF (52.5 ml) is treated with borane tetrahydrofuran complex (15.75 ml of a 1 M solution in THF, 15.75 mmol) and the resulting mixture stirred at RT for 72 hours. The mixture is then quenched with 1N HCl, stirred for 20 minutes, then extracted with DCM. Concentration and silica gel chromatography (10-50% EtOAc/hexanes) yields the title compound 1.87 g (87% yield) as colorless needles. ¹H NMR (500 MHz, CDCl₃) δ 7.55 (m, 1H), 7.04 (d, J9.4 Hz, 1H), 7.04 (d, J 8.3 Hz, 1H), 4.70 (d, 2H).

### Step B: 3-Fluoro-4-bromobenzyl bromide

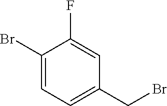

The product from step A was converted to the bromide according to the procedure from Intermediate 14. ¹H NMR (500 MHz, CDCl₃) δ 7.54 (m, 1H), 7.19 (d, 1H), 7.08 (d, 1H), 4.43 (s, 2H).

### Intermediate 16

### 5-(Bromomethyl)-1,3-difluoro-2-(methylthio)benzene

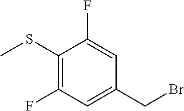

### Step A: 3,5-Difluoro-4-(methylthio)benzaldehyde

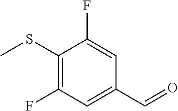

A solution of 3,4,5-trifluorobenzaldehyde (1.0 g, 6.3 mmol) in DMSO (7.8 mL) was treated with a slurry of sodium methanethiolate (0.438 g, 6.25 mmol) in DMSO (0.3 mL). The solution was warmed at 125° C. for 17 min in a microwave. Upon completion, the reaction was diluted with EtOAc and washed with H₂O (lx) and saturated aqueous NaCl (1×). The combined aqueous was back-extracted with EtOAc (3×), and the combined organic layer was dried over Na₂SO₄, filtered and evaporated in vacuo to yield a crude oil that was purified by silica gel column chromatography (0-30%, EtOAc-hexanes) to yield the title compound. ¹H NMR (500 MHz, CDCl₃) δ 9.88 (s, 1H), 7.39 (d, J7.0 Hz, 2H), 2.58 (s, 3H).

### Step B: [3,5-Difluoro-4-(methylthio)phenyl]methanol

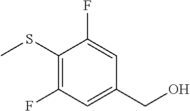

Sodium borohydride (25 mg, 0.66 mmol) was added to a 0° C. slurry of the product of step A (124 mg, 0.659 mmol) in methanol (4.39 mL). After 30 min the reaction mixture was warmed to room temperature, and at 1.5 h the reaction was diluted with DCM and quenched with 0.1 N HCl. The aqueous was extracted with DCM (2×) and then washed with saturated aqueous NaCl (1×). The combined organic layer was dried over Na₂SO₄, filtered and evaporated in vacuo to yield the title compound. ¹H NMR (500 MHz, CDCl₃) δ 6.81 (d, J 7.5 Hz, 2H), 4.55 (s, 2H), 3.23 (s, 1H), 2.37 (s, 3H).

### Step C: 5-(Bromomethyl)-1,3-difluoro-2-(methylthio)benzene

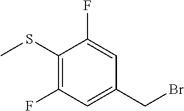

The product from step B was converted to the bromide according to the procedure from Intermediate 14. ¹H NMR (500 MHz, CDCl₃) δ 7.36 (d, J 7.5 Hz, 2H), 4.82 (s, 2H), 2.88 (s, 3H).

### Intermediate 17

### 3-Fluoro-4-(methylsulfonyl)phenol

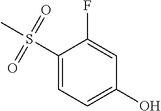

### Step A: (2-Fluoro-4-nitrophenyl)(methyl)sulfane

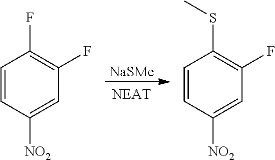

A mixture of the difluorobezene (60.0 g, 0.38 mol) and sodium methanethiolate (26.5 g, 0.38 mol) was stirred at room temperature overnight. The reaction mixture was poured into water (1.0 L) and extracted with EtOAc (300 mL×3). The combined organic layers were washed with brine, dried over Na₂SO₄ and concentrated. The residue was purified by column chromatography (PE: EtOAc=20:1˜15:1) to give the product as a yellow oil (35.0 g, yield: 50%). MS ESI calc'd. for C7H6FNO2S [M+H]⁺ 188. ¹H NMR (400 MHz, CDCl₃) δ 7.97 (d, 1H), 7.84 (d, 1H), 7.23 (t, 1H), 2.50 (s, 3H).

### Step B: 3-Fluoro-4-(methylthio)aniline

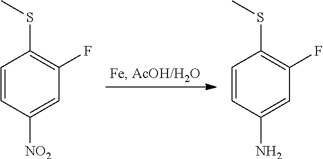

To a mixture of Step A product (10.0 g, 0.053 mol) in acetic acid (30 mL) and water (20 mL) was added iron (14.0 g, 0.26 mol), then the mixture was stirred at room temperature for 2 hours. TLC showed the reaction was complete. The reaction mixture was concentrated, EtOAc and aqueous sodium bicarbonate solution were added, and the mixture was filtered. The aqueous layer was extracted with EtOAc (200 mL×3), the combined organic portions were washed with brine, dried over Na₂SO₄ and concentrated to give the product as a white solid (8.0 g, yield: 95%). MS ESI calc'd. for C7H8FNS [M+H]⁺ 158. ¹H NMR (400 MHz, DMSO_d₆) δ 7.06 (t, 1H), 6.33 (d, 2H), 5.50 (s, 2H), 2.24 (s, 3H).

### Step C: 3-Fluoro-4-(methylthio)phenol

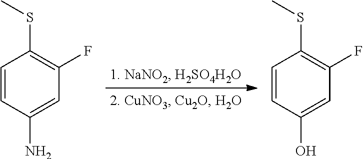

To a mixture of Step B product (4.0 g, 0.025 mol) and concentrated sulfuric acid (17.0 g, 0.17 mol) in water (250 mL) and THF (10 mL) was added solium nitrite (2.6 g, 0.037 mol) in water (10 mL) at 0° C., then the mixture was stirred at this temperature for 1 hour. The resulted mixture was added to a mixture of copper(II) nitrate trihydrate (3.6 g, 0.025 mol) and copper(I) oxide in water (300 mL) at 0° C. The reaction mixture was stirred for 15 min and extracted with EtOAc (300 mL×3). The combined organic portions were washed with brine, dried over Na₂SO₄ and concentrated to give the crude product as a brown oil (3.8 g, crude). ¹H NMR (400 MHz, DMSO_d₆) δ 9.99 (s, 1H), 7.22 (t, 1H), 6.60 (d, 2H), 2.34 (s, 3H). MS ESI [M+H]⁺ 159.

### Step D: 3-Fluoro-4-(methylsulfonyl)phenol

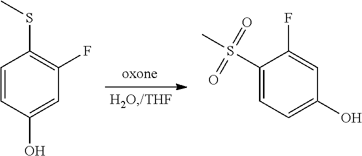

To a mixture of Step C product (3.8 g, crude) in THF (150 mL) was added oxone (30.0 g, 0.05 mol) in water (150 mL) at room temperature, then the mixture was stirred at this temperature for 2 hours. The reaction mixture extracted with EtOAc (100 mL×3), and the combined organic portions were washed with brine, dried over Na₂SO₄ and concentrated. The residue was purified by column chromatography (PE: EtOAc=3:1˜1:1) to give the desired product as a white solid (2.6 g, yield: 55% for 2 steps). ¹H NMR (400 MHz, DMSO_d₆) δ 9.99 (s, 1H), 7.22 (t, 1H), 6.60 (d, 2H), 2.34 (s, 3H). MS ESI [M+H]⁺ 191.

### Intermediate 18

### 4-(Ethylsulfonyl)-2-fluorophenol

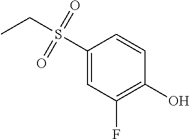

### Step A: (3,4-Difluorophenyl)(ethyl)sulfane

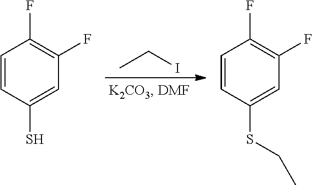

A mixture of 3,4-difluorothiophenol (4.8 g, 33 mmol), ethyl iodide (5.5 g, 35 mmol) and K₂CO₃ (5.5 g, 40 mmol) in DMF (50 mL) was stirred at room temperature for 3 h. TLC showed the reaction was complete. The reaction mixture was poured into water (200 mL) and extracted with EtOAc (100 mL×3). The combined organic layers were washed with brine, dried over Na₂SO₄ and concentrated to give the product as a colorless oil (5.1 g, yield: 89%). ¹H NMR (400 MHz, CDCl3) δ 7.06-7.24 (m, 3H), 2.89 (q, 2H), 1.29 (t, 3H). MS ESI [M+H]⁺ 175.

### Step B: 4-(Ethylsulfonyl)-1,2-difluorobenzene

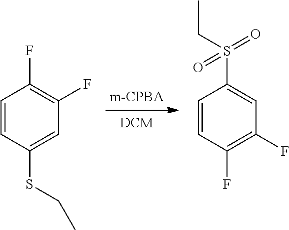

To a stirred suspension of Step A product (5.1 g, 29 mmol) in DCM (100 mL) was added portionwise m-CPBA (10 g, 58 mmol). The reaction mixture was stirred at room temperature overnight. TLC showed the reaction was complete. The reaction was filtered and the filtrate was diluted with DCM (300 mL), then washed with aqueous NaS₂SO₄ and brine, dried over Na₂SO₄ and concentrated to give the product as a colorless oil (5.0 g, yield: 83%). ¹H NMR (400 MHz, CDCl3) δ 7.72-7.68 (m, 2H), 7.41-7.26 (m, 1H), 3.12 (q, 2H), 1.28 (t, 3H). MS ESI [M+H]⁺ 207.

### Step C: 4-(Ethylsulfonyl)-2-fluoro-1-methoxybenzene

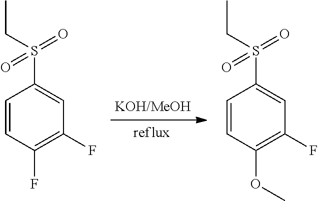

To a stirred suspension of Step B product (5.0 g, 24 mmol) in MeOH (50 mL) was added KOH (1.6 g, 29 mmol). The reaction mixture was heated under reflux for 1 h, then cooled to ambient temperature and filtered. The filtrate was washed with brine, dried over Na₂SO₄ and concentrated to give the product as a white solid (4.8 g, yield: 90%). ¹H NMR (400 MHz, CDCl3) δ 7.67 (d, 1H), 7.59 (d, 1H), 3.97 (s, 3H), 3.10 (q, 2H), 1.26 (t, 3H). MS ESI [M+H]⁺ 219.

### Step D: 4-(Ethylsulfonyl)-2-fluorophenol

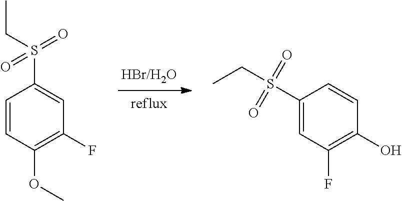

A mixture of Step C product (2.5 g, 11 mmol) in 30% of HBr/H₂O (50 mL) was refluxed at 100° C. overnight. The reaction mixture was poured into water (50 mL) and extracted with EtOAc (30 mL×3). The combined organic layers were washed with brine, dried over Na₂SO₄ and concentrated to give the product as a white solid (2.0 g, yield: 87%). ¹H NMR (400 MHz, DMSO_d₆) δ 11.2 (br, 1H), 7.63 (d, J=10.8 Hz, 1H), 7.53 (d, 1H), 7.14 (t, 1H), 3.23 (q, 2H), 1.06 (t, 3H). MS ESI [M+H]⁺ 205.

### Intermediate 19

### 3-Fluoro-4-(1H-tetrazol-1-yl)phenol

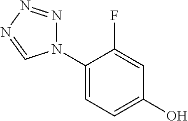 [SMILES: Oc1ccc(-n2cnnn2)c(F)c1]

### Step A: 3-Fluoro-4-(1H-tetrazol-1-yl)phenol

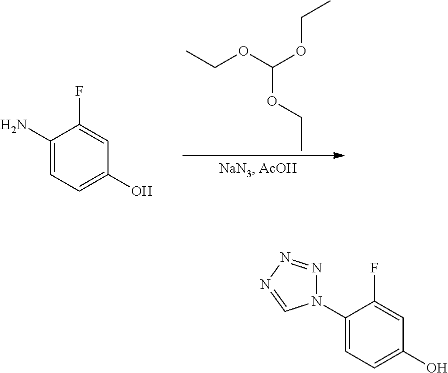

To a solution of the aniline (2.54 g, 20 mmol) and triethyl orthoformate (4.74 g, 32 mmol) in AcOH (36 mL) was added NaN₃ (1.95 g, 30 mmol). The mixture was refluxed for 16 h. The reaction solution was cooled to room temperature, diluted with water, neutralized with Na₂CO₃ and extracted with EtOAc. The organic layer was washed with brine, dried over Na₂SO₄ and concentrated. The residue was purified by column chromatography (DCM: MeOH=30:1) to give the product as a red solid (2.9 g, yield: 81%). ¹H NMR (400 MHz, MeOD) δ 9.49-9.50 (d, 1H), 7.55-7.60 (t, 1H), 6.83 (s, 1H), 6.81 (s, 1H). MS ESI [M+H]⁺ 181.

### Intermediate 20

### 2-Fluoro-4-(1H-tetrazol-1-yl)phenol

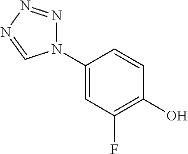 [SMILES: Oc1ccc(-n2cnnn2)cc1F]

### Step A: 2-Fluoro-4-(1H-tetrazol-1-yl)phenol

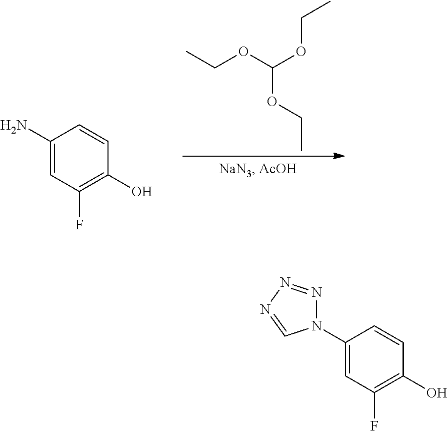

A mixture of the aniline (5 g, 39.4 mmol), HC(OEt)₃ (17.4 g, 118.1 mmol) and NaN₃ (3.8 g, 59.1 mmol) in HOAc (80 mL) was stirred at 140° C. for 5 hours. Then the mixture was adjusted to pH=12, and extracted with EtOAc (100 mL×3). The organic layers were dried over anhydrous Na₂SO₄, concentrated and purified by column chromatography (PE: EtOAc=10:1˜3:1) to give the compound as a yellow solid (4.0 g, yield: 56%). ¹H NMR (400 MHz, MeOH) δ 9.66 (s, 1H), 7.69-7.65 (m, 1H), 7.54-7.50 (m, 1H), 7.16-7.12 (m, 1H).

### Intermediate 21

### 1-(2-Fluoro-4-hydroxyphenyl)imidazolidin-2-one

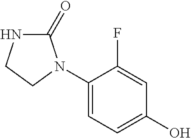 [SMILES: O=C1NCCN1c1ccc(O)cc1F]

### Step A: 1-(2-Chloroethyl)-3-(2-fluoro-4-hydroxyphenyl)urea

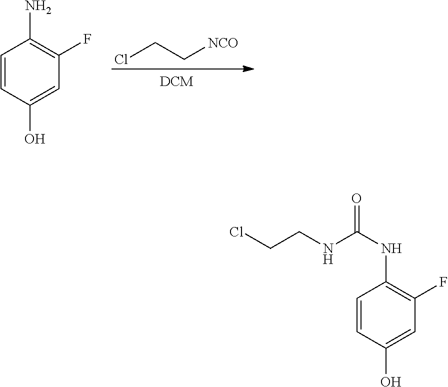

To a solution of the aniline (1.7 g, 13.4 mmol) in DCM (80 mL) was added 1-Chloro-2-isocyanato-ethane (2.1 g, 20.1 mmol) at ambient temperature. The mixture was stirred at reflux for 2 h. The reaction solution was cooled to room temperature and filtered to give the product (filter cake) as a white solid (2.0 g, crude). MS ESI [M+H]⁺ 233.

### Step B: 1-(2-Fluoro-4-hydroxyphenyl)imidazolidin-2-one

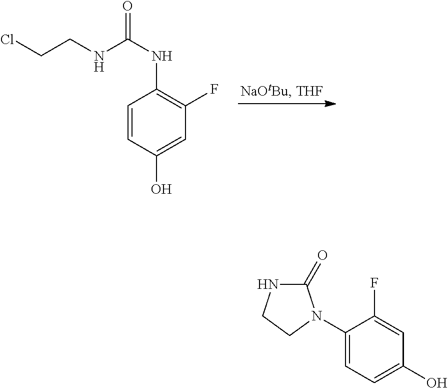 [SMILES: O=C(NCCCl)Nc1ccc(O)cc1F ; O=C1NCCN1c1ccc(O)cc1F]

To a solution of Step A product (2.0 g, 8.6 mmol) in THF (50 mL) was added dropwise a solution of NaO^{t}Bu (5.0 g, 52 mmol) in THF (100 mL) at room temperature under N₂. The mixture was stirred for 1 h at room temperature and quenched with formic acid (3.8 mL). The reaction solution was concentrated, dissolved in DMF and filtered. The filtrate was concentrated, and the residue was purified by column chromatography (DCM: MeOH=40:1˜20:1) to give the product as a red solid (600 mg, yield: 36%). MS ESI [M+H]⁺ 197.

### Intermediate 22

### 1-(3-Fluoro-4-hydroxyphenyl)imidazolidin-2-one

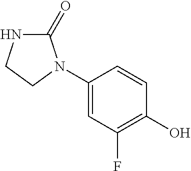 [SMILES: O=C1NCCN1c1ccc(O)c(F)c1]

### Step A: 1-(3-Fluoro-4-hydroxyphenyl)imidazolidin-2-one

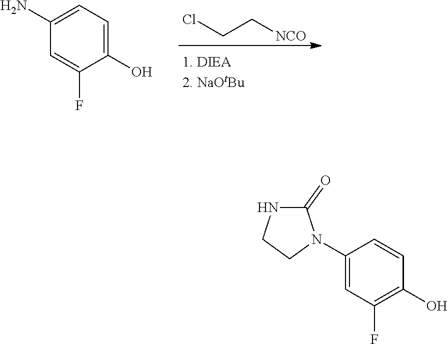

To a solution of the aniline (1.7 g, 13.4 mmol) in DCM (80 mL) was added 1-Chloro-2-isocyanato-ethane (2.1 g, 20.1 mmol) at ambient temperature. The mixture was stirred at reflux for 2 h. The reaction solution was cooled room temperature and added dropwise to a solution of NaOtBu (5.0 g, 52 mmol) in THF (100 mL) at room temperature under N₂. The mixture was stirred for 1 h at room temperature and quenched with formic acid (3.8 mL). The reaction solution was concentrated, dissolved in DMF and filtered. The filtrate was concentrated, and the residue was purified by column chromatography (DCM: MeOH=40:1-20:1) to give the product as a red solid (600 mg, yield: 36%). MS ESI [M+H]⁺ 197.

### Intermediate 23

### 1-(Azetidin-1-yl)-2-(6-chloropyridin-3-yl)ethanone

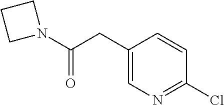 [SMILES: O=C(Cc1ccc(Cl)nc1)N1CCC1]

### Step A: 1-(Azetidin-1-yl)-2-(6-chloropyridin-3-yl)ethanone

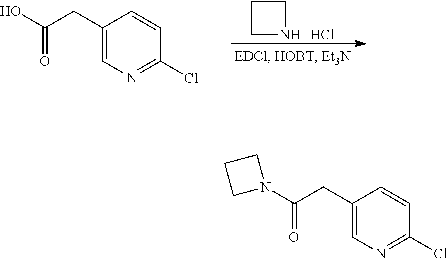

A mixture of the pyridyl acetic acid (1.7 g, 10 mmol), azetidine HCl salt (1 g, 11 mmol), HOBT (1.62 g, 12 mmol), EDCl (2.3 g, 12 mmol), and TEA (4.2 mL) in DCM (25 mL) was stirred at room temperature for 16 hours Then the reaction mixture was concentrated and the residue was purified by column chromatography (PE: EtOAc=1:1˜0:1) to give the product. MS ESI [M+H]⁺ 211.

### Intermediate 24

### 1-(Azetidin-1-yl)-2-(2-fluoro-4-hydroxyphenyl)ethanone

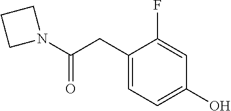 [SMILES: O=C(Cc1ccc(O)cc1F)N1CCC1]

### Step A: 1-(Azetidin-1-yl)-2-(2-fluoro-4-hydroxyphenyl)ethanone

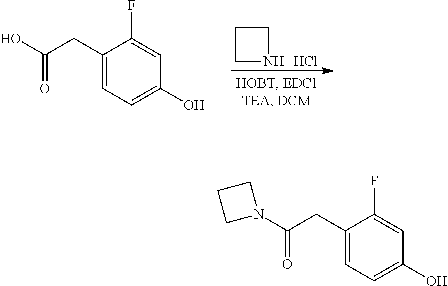

A mixture of of the phenyl acetic acid (1.7 g, 10 mmol), azetidine HCl salt (1 g, 11 mmol), HOBT (1.62 g, 12 mmol), EDCl (2.3 g, 12 mmol), and TEA (4.2 mL) in DCM (25 mL) was stirred at room temperature for 16 hours. Then the reaction mixture was concentrated and the residue was purified by column chromatography (PE: EtOAc=1:1˜0:1) to give the product (500 mg, yield: 26%). MS ESI [M+H]⁺ 210.

### Intermediate 25

### 2-(2-Fluoro-4-hydroxyphenyl)-N,N-dimethylacetamide

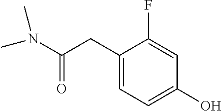

### Step A: 2-(2-Fluoro-4-hydroxyphenyl)-N,N-dimethylacetamide

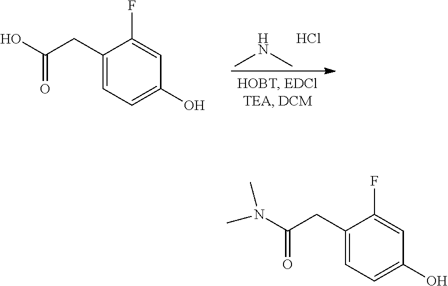

A mixture of the phenyl acetic acid (510 mg, 3 mmol), dimethyl-amine HCl salt (250 mg, 3.6 mmol), HOBT (810 mg, 6 mmol), EDCl (1.15 g, 6 mmol), and TEA (1.4 mL) in DCM (15 mL) was stirred at room temperature for 16 hours. Then the reaction mixture was concentrated and the residue was purified by column chromatography (PE: EtOAc=1:1˜0:1) to give the product (520 mg, yield: 82%). ¹H NMR (400 MHz, MeOH) δ 7.06 (dd, 1H), 6.63-6.50 (dd, 2H), 3.65 (s, 2H), 3.13 (s, 3H), 2.98 (s, 3H). MS ESI [M+H]⁺ 198.

### Intermediate 26

### 1-(Azetidin-1-yl)-2-(3-fluoro-4-hydroxyphenyl)ethanone

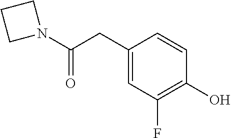 [SMILES: O=C(Cc1ccc(O)c(F)c1)N1CCC1]

### Step A: 1-(Azetidin-1-yl)-2-(3-fluoro-4-hydroxyphenyl)ethanone

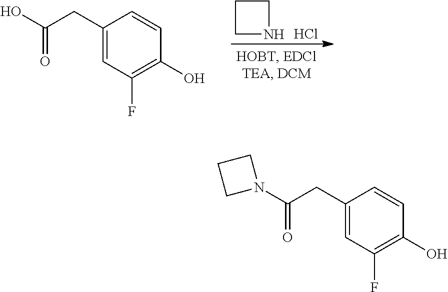

A mixture of the phenyl acetic acid (1.7 g, 10 mmol), azetidine HCl salt (1 g, 11 mmol), HOBT (1.62 g, 12 mmol), EDCl (2.3 g, 12 mmol), TEA (4.2 mL) in DCM (25 mL) was stirred at room temperature for 16 hours. Then the reaction mixture was concentrated and the residue was purified by column chromatography (PE: EtOAc=1:1˜0:1) to give the desired product (600 mg, yield: 28%). ¹H NMR (400 MHz, MeOH) δ 6.99 (d, 1H), 6.87 (broad s, 2H), 4.25 (t, 2H), 4.05 (t, 2H), 3.38 (s, 2H), 2.29 (m, 2H). MS ESI [M+H]⁺ 210.

### Intermediate 27

### 1-(Azetidin-1-yl)-2-(2,4,6-trifluorophenyl)ethanone

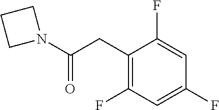 [SMILES: O=C(Cc1c(F)cc(F)cc1F)N1CCC1]

### Step A: 1-(Azetidin-1-yl)-2-(2,4,6-trifluorophenyl)ethanone

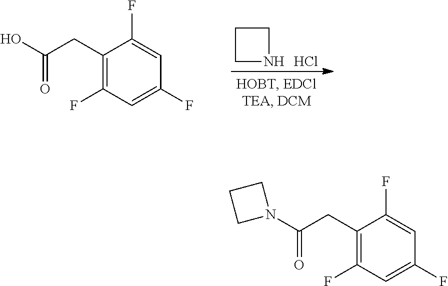

A mixture of the phenyl acetic acid (1.0 g, 5.3 mmol), azetidine HCl salt (0.5 g, 5.3 mmol), HOBT (1.62 g, 12 mmol), EDCl (2.3 g, 12 mmol), and TEA (4.2 mL) in DCM (25 mL) was stirred at room temperature for 16 hours. Then the reaction mixture was concentrated and the residue was purified by column chromatography (PE: EtOAc=1:1˜0:1) to give the desired product (700 mg, yield: 58%). MS ESI [M+H]⁺ 230.

### Intermediate 28

### N,N-dimethyl-2-(2,4,6-trifluorophenyl)acetamide

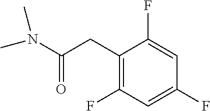

### Step A: N,N-dimethyl-2-(2,4,6-trifluorophenyl)acetamide

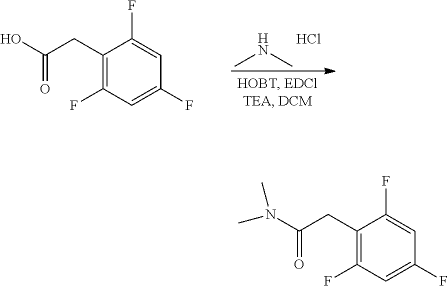

A mixture of the phenyl acetic acid (1.0 g, 5.3 mmol), dimethylamine HCl salt (0.3 g, 5.3 mmol), HOBT (1.62 g, 12 mmol), EDCl (2.3 g, 12 mmol), and TEA (4.2 mL) in DCM (25 mL) was stirred at room temperature for 16 hours. Then the reaction mixture was concentrated and the residue was purified by column chromatography (PE: EtOAc=1:1˜0:1) to give the desired product (700 mg, yield: 58%). MS ESI [M+H]⁺ 218.

### Intermediate 29

### 1-(Azetidin-1-yl)-2-(2,6-difluoro-4-hydroxyphenyl)ethanone

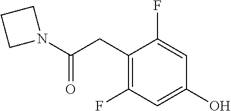 [SMILES: O=C(Cc1c(F)cc(O)cc1F)N1CCC1]

### Step A: 2-(2,6-Difluoro-4-hydroxyphenyl)acetic acid

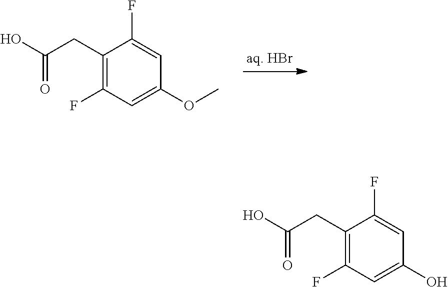

To a solution of the methyl ether (2.0 g, 9.9 mmol) in AcOH (10 mL) was added HBr (10 mL). Then the mixture was stirred at 100° C. over night. Then the reaction mixture was concentrated to give the desired product.

### Step B: 1-(Azetidin-1-yl)-2-(2,6-difluoro-4-hydroxyphenyl)ethanone

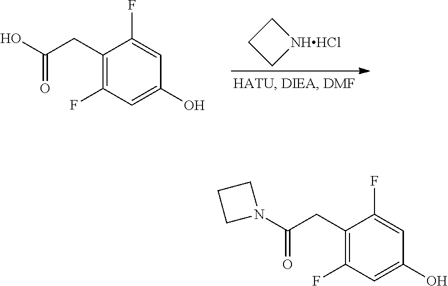

A mixture of the Step A product (1.0 g, 5.3 mmol), azetidine HCl salt (0.5 g, 5.3 mmol), HOBT (1.62 g, 12 mmol), EDCl (2.3 g, 12 mmol), and TEA (4.2 mL) in DCM (25 mL) was stirred at room temperature for 16 hours. Then the reaction mixture was concentrated and the residue was purified by column chromatography (PE: EtOAc=1:1˜0:1) to give the desired product (700 mg, yield: 58%). MS ESI [M+H]⁺ 228.

### Intermediate 30

### 2-(2,6-Difluoro-4-hydroxyphenyl)-N,N-dimethylacetamide

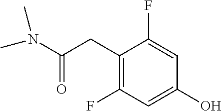

### Step A: 2-(2,6-Difluoro-4-hydroxyphenyl)-N,N-dimethylacetamide

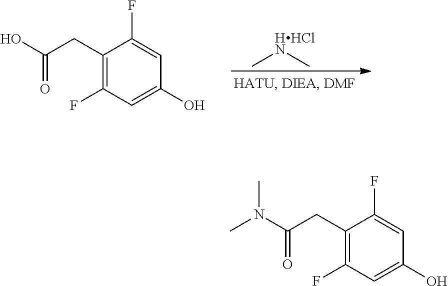

A mixture of the phenyl acetic acid (Step A product of Intermediate 29, 1.0 g, 5.3 mmol), dimethylamine HCl salt (0.3 g, 5.3 mmol), HOBT (1.62 g, 12 mmol), EDCl (2.3 g, 12 mmol), and TEA (4.2 mL) in DCM (25 mL) was stirred at room temperature for 16 hours. Then the reaction mixture was concentrated and the residue was purified by column chromatography (PE: EtOAc=1:1˜0:1) to give the desired product (700 mg, yield: 58%). MS ESI [M+H]⁺ 216.

### EXAMPLES

### Examples 1-13

### Preparation of 5-chloro-2-(4-((1R,2S)-2-(((5-(methylsulfonyl)pyridin-2-yl)methoxy)methyl)cyclopropyl)piperidin-1-yl)pyrimidine

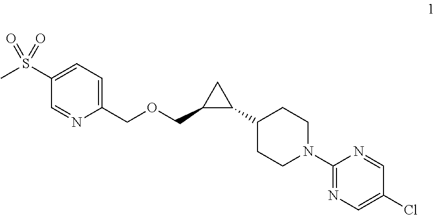 [SMILES: CS(=O)(=O)c1ccc(COC[C@H]2C[C@@H]2C2CCN(c3ncc(Cl)cn3)CC2)nc1]

### Step A: tert-Butyl 4-((1R,2S)-2-(((5-bromopyridin-2-yl)methoxy)methyl)cyclopropyl)piperidine-1-carboxylate

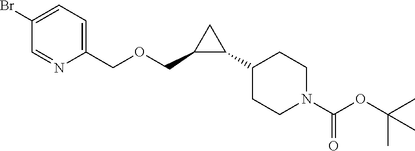 [SMILES: CC(C)(C)OC(=O)N1CCC([C@H]2C[C@@H]2COCc2ccc(Br)cn2)CC1]

Sodium hydride (197 mg, 4.93 mmol) was added to a stirring solution of tert-butyl 4-((1R,2S)-2-(hydroxymethyl)cyclopropyl)piperidine-1-carboxylate (Intermediate 3) (840 mg, 3.29 mmol) in DMF (10 mL) that had been cooled to 0° C. in an ice bath and placed under an inert atmosphere. 10 min later 5-bromo-2-(bromomethyl)pyridine (Intermediate 14) (990 mg, 3.95 mmol) was introduced to this mixture. The ice bath was removed and the reaction warmed to rt. The reaction was aged for 1 hr then diluted with EtOAc (20 mL) and neutralized by the slow addition of saturated aqueous ammonium chloride solution (20 mL). The layers were cut and the aqueous phase extracted with EtOAc (20 mL×2). The combined organic layers were dried over MgSO₄, filtered, and concentrated under reduced pressure. The residue was loaded onto a silica column (KP-Sil 50 g SNAP column, Biotage system) eluting with a range of 0-25% EtOAc/Hex over 12 CV to give the desired compound (1.23 g, 88%). LC/MS (m/z): 425 (M+H)⁺.

### Step B: tert-Butyl 4-((1R,2S)-2-(((5-(methylthio)pyridin-2-yl)methoxy)methyl)cyclopropyl)piperidine-1-carboxylate

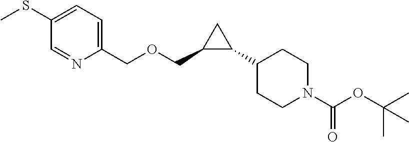 [SMILES: CSc1ccc(COC[C@H]2C[C@@H]2C2CCN(C(=O)OC(C)(C)C)CC2)nc1]

A 2.5 M solution of n-butyllithium in hexanes (1.39 mL, 3.47 mmol) was slowly introduced to a solution of tert-butyl 4-((1R,2S)-2-(((5-bromopyridin-2-yl)methoxy)methyl)cyclopropyl)piperidine-1-carboxylate (1.23 g, 2.89 mmol) in anhydrous THF (9 mL) that had been cooled to −78° C. and placed under an inert atmosphere. After 10 min dimethyl disulfide (330 uL, 3.76 mmol) was added to the reaction mixture. The reaction was aged at −78° C. for 2 hrs then quenched with saturated aqueous ammonium chloride solution (15 mL) and diluted with EtOAc (15 mL). The layers were cut and the aqueous phase extracted with EtOAc (20 mL×2). The combined organic layers were dried over MgSO₄, filtered, and concentrated under reduced pressure. The residue was loaded onto a silica column (KP-Sil 25 g SNAP column, Biotage system) eluting with a range of 3-7% MeOH/DCM over 12 CV to give the desired compound (1.04 g, 92%). LC/MS (m/z): 393 (M+H)⁺.

### Step C: tert-Butyl 4-((1R,2S)-2-(((5-(methylsulfonyl)pyridin-2-yl)methoxy)methyl)cyclopropyl)piperidine-1-carboxylate

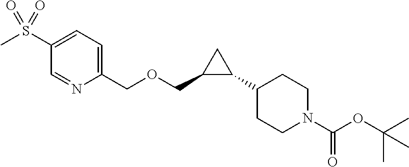 [SMILES: CC(C)(C)OC(=O)N1CCC([C@H]2C[C@@H]2COCc2ccc(S(C)(=O)=O)cn2)CC1]

A solution of oxone (3.26 g, 5.3 mmol) in water (15 mL) was added to a solution of tert-butyl 4-((1R,2S)-2-(((5-(methylthio)pyridin-2-yl)methoxy)methyl)cyclopropyl)piperidine-1-carboxylate (1.04 g, 2.65 mmol) in MeOH (10 mL) and the resulting mixture aged at rt for 1 hr. The reaction mixture was diluted with DCM (30 mL) and water (20 mL), the layers cut, and the aqueous phase extracted with DCM (20 mL×2). The combined organic layers were dried over MgSO₄, filtered, and concentrated under reduced pressure. The residue was loaded onto a silica column (KP-Sil 25 g SNAP column, Biotage system) eluting with a range of 20-70% EtOAc/Hex over 12 CV to give the desired compound (650 mg, 58%). LC/MS (m/z): 425 (M+H)⁺.

### Step D: 5-(Methylsulfonyl)-2-((((1S,2R)-2-(piperidin-4-yl)cyclopropyl)methoxy)methyl)pyridine hydrochloride

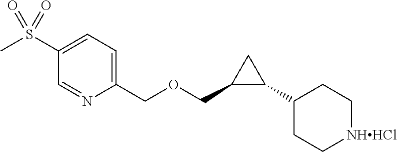

A solution of 4 M HCl in dioxane (4 mL, 16 mmol) was added to a solution of tert-butyl 4-((1R,2S)-2-(((5-(methylsulfonyl)pyridin-2-yl)methoxy)methyl)cyclopropyl)piperidine-1-carboxylate (650 mg, 1.53 mmol) in DCM (4 mL). This mixture was stirred at rt for 1 hr. The reaction mixture was subsequently concentrated under reduced pressure to afford the title compound (505 mg, 92%) as a crude product to be used for the next step. LC/MS (m/z): 325 (M+H)⁺.

### Step E: 5-Chloro-2-(4-((1R,2S)-2-(((5-(methylsulfonyl)pyridin-2-yl)methoxy)methyl)cyclopropyl)piperidin-1-yl)pyrimidine

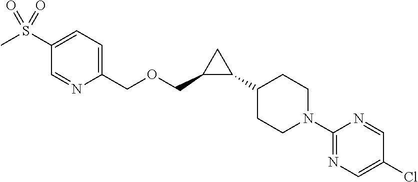 [SMILES: CS(=O)(=O)c1ccc(COC[C@H]2C[C@@H]2C2CCN(c3ncc(Cl)cn3)CC2)nc1]

Cesium carbonate (185 mg, 0.56 mmol) was added to a solution of 5-(methylsulfonyl)-2-((((1S,2R)-2-(piperidin-4-yl)cyclopropyl)methoxy)methyl)pyridine hydrochloride (78 mg, 0.216 mmol) and 2,5-dichloropyrimidine (42 mg, 0.28 mmol) in DMF (1 mL). The resulting mixture was stirred and heated at 55° C. overnight. The reaction mixture was cooled to rt and diluted with EtOAc (5 mL) and water (5 mL). The layers were cut and the aqueous phase extracted with EtOAc (5 mL×2). The combined organic layers were dried over MgSO₄, filtered, and concentrated under reduced pressure. The residue was loaded onto 2×2000 micron silica preparative TLC plates (uv 254 active) which were developed using 5% MeOH/DCM as the solvent system. The desired silica (Rf=0.3 @ 5% MeOH/DCM) was collected and extracted to give the title compound (24 mg, 25%). ¹H NMR (500 MHz, CD₃CN) δ 8.99 (s, 1H), 8.24 (s, 2H), 8.22 (d, 1H), 7.67 (d, 1H), 4.69 (s, 2H), 4.62 (q, 2H), 3.42 (dq, 2H), 3.12 (s, 3H), 2.85 (t, 2H), 1.81 (dd, 2H), 1.27 (td, 2H), 1.01-0.90 (m, 2H), 0.54 (h, 1H), 0.47-0.38 (m, 2H). LC/MS (m/z): 437 (M+H)⁺, GPR119 Human EC₅₀: 3.8 nM (HTRF assay).

The examples in Table 1 were synthesized according to the methods described in Example 1 employing Intermediates 3, 9, and 15 in addition to commercially available starting materials.

TABLE 1

| Example | | Observed | GPR119 |
|---|---|---|---|
| # | Chemical Structure | Mass | Human EC₅₀ |
| 2 | 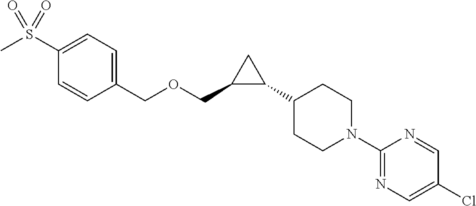 [SMILES: CS(=O)(=O)c1ccc(COC[C@H]2C[C@@H]2C2CCN(c3ncc(Cl)cn3)CC2)cc1] | 436 [M + H]⁺ | 1.2 nM HTRF assay |
| 3 | 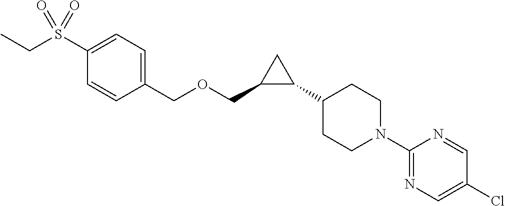 [SMILES: CCS(=O)(=O)c1ccc(COC[C@H]2C[C@@H]2C2CCN(c3ncc(Cl)cn3)CC2)cc1] | 449 [M + H]⁺ | 0.73 nM HTRF assay |
| 4 | 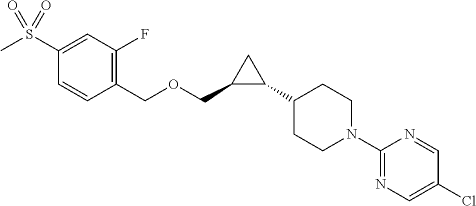 [SMILES: CS(=O)(=O)c1ccc(COC[C@H]2C[C@@H]2C2CCN(c3ncc(Cl)cn3)CC2)c(F)c1] | 454 [M + H]⁺ | 1.6 nM LCMP assay |
| 5 | 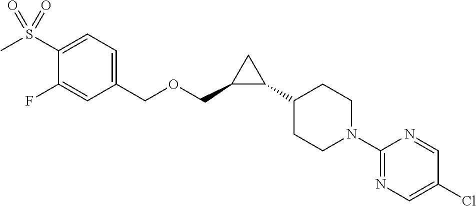 [SMILES: CS(=O)(=O)c1ccc(COC[C@H]2C[C@@H]2C2CCN(c3ncc(Cl)cn3)CC2)cc1F] | 454 [M + H]⁺ | 1.0 nM HTRF assay |
| 6 | 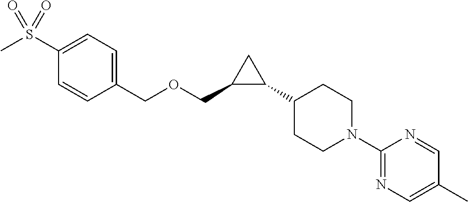 | 416 [M + H]⁺ | 7.1 nM HTRF assay |
| 7 | 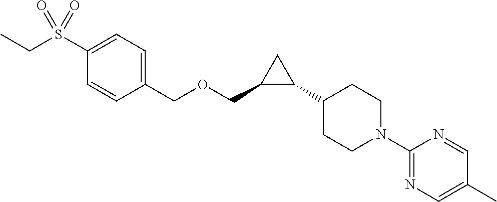 [SMILES: CCS(=O)(=O)c1ccc(COC[C@H]2C[C@@H]2C2CCN(c3ncc(C)cn3)CC2)cc1] | 430 [M + H]⁺ | 5.8 nM HTRF assay |
| 8 | 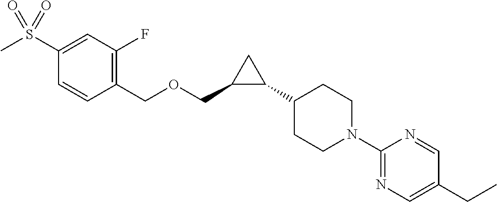 [SMILES: CCc1cnc(N2CCC([C@H]3C[C@@H]3COCc3ccc(S(C)(=O)=O)cc3F)CC2)nc1] | 448 [M + H]⁺ | 1.3 nM LCMP assay |
| 9 | 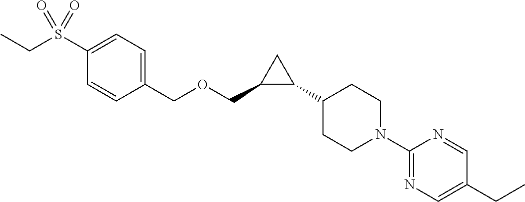 [SMILES: CCc1cnc(N2CCC([C@H]3C[C@@H]3COCc3ccc(S(=O)(=O)CC)cc3)CC2)nc1] | 444 [M + H]⁺ | 22 nM LCMP assay |
| 10 | 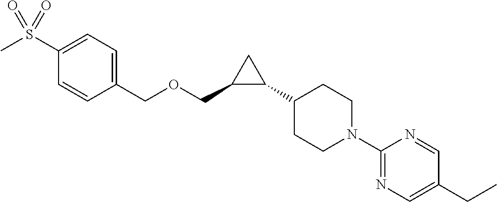 [SMILES: CCc1cnc(N2CCC([C@H]3C[C@@H]3COCc3ccc(S(C)(=O)=O)cc3)CC2)nc1] | 430 [M + H]⁺ | 1.9 nM HTRF assay |
| 11 | 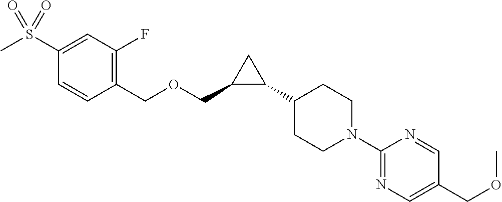 | 464 [M + H]⁺ | 7.1 nM LCMP assay |
| 12 | 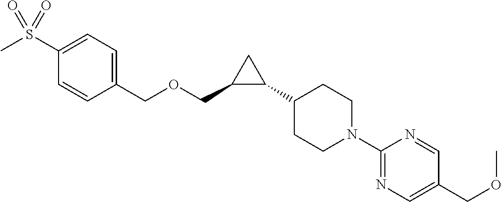 | 446 [M + H]⁺ | 14 nM LCMP assay |
| 13 | 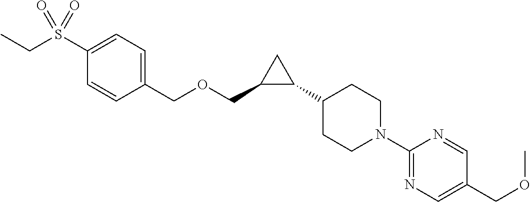 [SMILES: CCS(=O)(=O)c1ccc(COC[C@H]2C[C@@H]2C2CCN(c3ncc(COC)cn3)CC2)cc1] | 460 [M + H]⁺ | 24 nM LCMP assay |

### Examples 14-20

### Preparation of 1-methylcyclopropyl 4-((1R,2S)-2-((2-fluoro-4-(methylsulfonyl)benzyloxy)methyl)cyclopropyl)piperidine-1-carboxylate

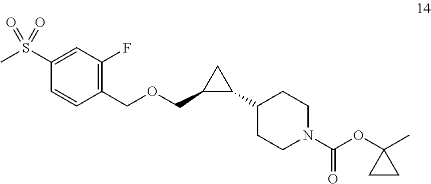

### Step A: 4-((1R,2S)-2-((2-Fluoro-4-(methylsulfonyl)benzyloxy)methyl)cyclopropyl)piperidine hydrochloride

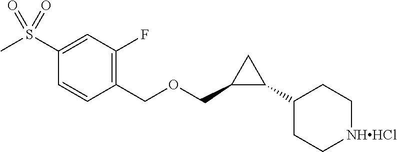

This intermediate was generated by following Steps A-D described in the synthesis of Example 1 using Intermediate 3 and commercial starting materials. LC/MS (m/z): 342 (M+H)⁺.

### Step B: 1-Methylcyclopropyl 4-((1R,2S)-2-((2-fluoro-4-(methylsulfonyl)benzyloxy)methyl)cyclopropyl)piperidine-1-carboxylate

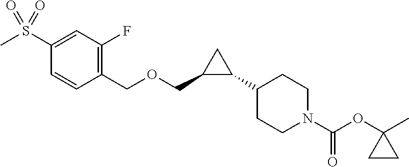

A solution of 4-((1R,2S)-2-((2-fluoro-4-(methylsulfonyl)benzyloxy)methyl)cyclopropyl)piperidine hydrochloride (60 mg, 0.159 mmol), 2,5-dioxopyrrolidin-1-yl 1-methylcyclopropyl carbonate (Intermediate 12) (44 mg, 0.206 mmol), and triethylamine (55 uL, 0.40 mmol) in anhydrous ACN (1.5 mL) was stirred at rt for 1 hr. The reaction mixture was concentrated under reduced pressure then diluted with EtOAc (5 mL) and water (5 mL). The layers were cut and the aqueous phase extracted with EtOAc (5 mL×2). The combined organic layers were dried over MgSO₄, filtered, and concentrated under reduced pressure. The residue was loaded onto 2×2000 micron silica preparative TLC plates (uv 254 active) which were developed using 50% EtOAc/Hex as the solvent system. The desired silica (Rf=0.5 @ 50% EtOAc/Hex) was collected and extracted to give the title compound (42 mg, 60%). ¹H NMR (500 MHz, CD₃CN) δ 7.74 (t, 1H), 7.71 (d, 1H), 7.65 (d, 1H), 4.63 (s, 2H), 4.09-3.84 (m, 2H), 3.38 (qd, 2H), 3.08 (s, 3H), 2.66 (t, 2H), 1.68 (dd, 2H), 1.49 (s, 3H), 1.18 (pd, 2H), 0.96-0.88 (m, 1H), 0.83-0.74 (m, 3H), 0.58 (t, 2H), 0.53-0.48 (m, 1H), 0.42-0.34 (m, 2H). LC/MS (m/z): 440 (M+H)⁺, GPR119 Human EC₅₀: 8.8 nM (LCMP assay).

The examples in Table 2 were synthesized according to the methods described in Example 14 employing Intermediates 3, 11-13, and 15 in addition to commercially available starting materials.

TABLE 2

| Example | | Observed | GPR119 |
|---|---|---|---|
| # | Chemical Structure | Mass | Human EC₅₀ |
| 15 | 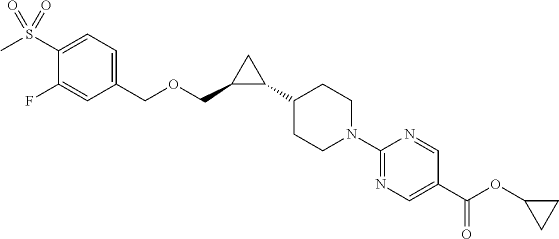 | 426 [M + H]⁺ | 2.4 nM HTRF assay |
| 16 | 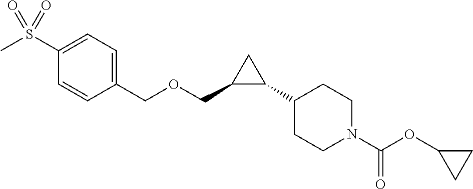 [SMILES: CS(=O)(=O)c1ccc(COC[C@H]2C[C@@H]2C2CCN(C(=O)OC3CC3)CC2)cc1] | 408 [M + H]⁺ | 5.7 nM HTRF assay |
| 17 | 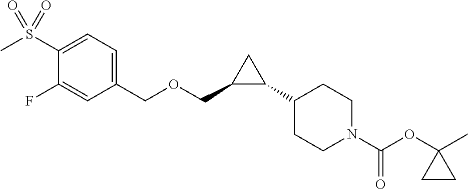 | 440 [M + H]⁺ | 4.6 nM HTRF assay |
| 18 | 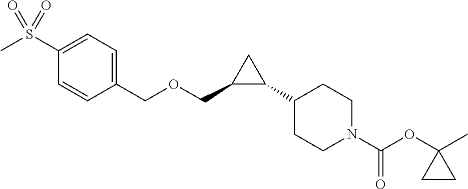 | 422 [M + H]⁺ | 6.4 nM HTRF assay |
| 19 | 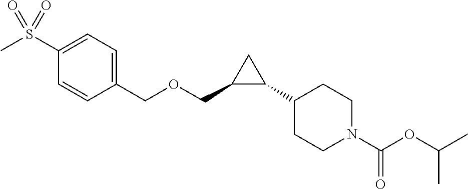 [SMILES: CC(C)OC(=O)N1CCC([C@H]2C[C@@H]2COCc2ccc(S(C)(=O)=O)cc2)CC1] | 410 [M + H]⁺ | 9.1 nM HTRF assay |
| 20 | 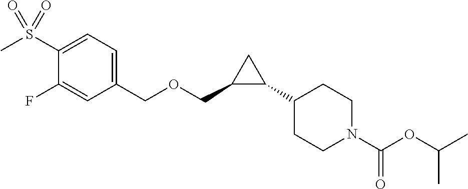 [SMILES: CC(C)OC(=O)N1CCC([C@H]2C[C@@H]2COCc2ccc(S(C)(=O)=O)c(F)c2)CC1] | 428 [M + H]⁺ | 5.8 nM HTRF assay |

### Examples 21-22

### Preparation of 3-isopropyl-5-(4-((1R,2S)-2((4-(methylsulfonyl)benzyloxy)methyl)cyclopropyl)piperidin-1-yl)-1,2,4-oxadiazole

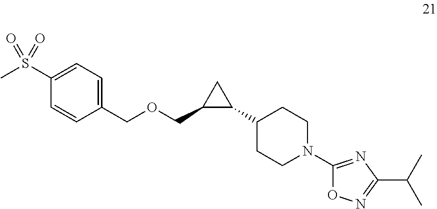 [SMILES: CC(C)c1noc(N2CCC([C@H]3C[C@@H]3COCc3ccc(S(C)(=O)=O)cc3)CC2)n1]

### Step A: 4-((1R,2S)-2-((4-(Methylsulfonyl)benzyloxy)methyl)cyclopropyl)piperidine hydrochloride

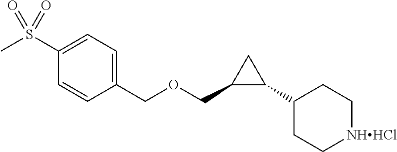

This intermediate was generated by following Steps A-D described in the synthesis of Example 1 using Intermediate 3 and commercially available starting materials. LC/MS (m/z): 324 (M+H)⁺.

### Step B: 4-((1R,2S)-2-((4-(Methylsulfonyl)benzyloxy)methyl)cyclopropyl)piperidine-1-carbonitrile

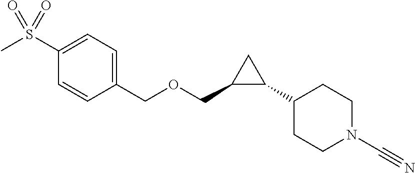 [SMILES: CS(=O)(=O)c1ccc(COC[C@H]2C[C@@H]2C2CCN(C#N)CC2)cc1]

A solution of 3 M cyanogen bromide in DCM (430 uL, 1.3 mmol) was added to a preformed mixture of 4-((1R,2S)-2-((4-(methylsulfonyl)benzyloxy)methyl)cyclopropyl)piperidine hydrochloride (400 mg, 1.11 mmol) and sodium bicarbonate (220 mg, 2.6 mmol) in DCM (5 mL) and water (5 mL) that had been cooled to 0° C. The reaction was stirred at 0° C. for 30 min then warmed to rt and stirred for 2 hrs. The layers were cut and the aqueous phase extracted with DCM (5 mL×2). The combined organic layers were dried over MgSO₄, filtered, and concentrated under reduced pressure to afford the title compound (360 mg, 92%) as a crude product to be used for the next step. LC/MS (m/z): 349 (M+H)⁺.

### Step C: 3-Isopropyl-5-(4-((1R,2S)-2-((4-(methylsulfonyl)benzyloxy)methyl)cyclopropyl)piperidin-1-yl)-1,2,4-oxadiazole

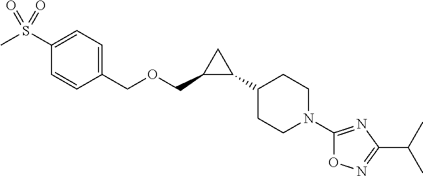 [SMILES: CC(C)c1noc(N2CCC([C@H]3C[C@@H]3COCc3ccc(S(C)(=O)=O)cc3)CC2)n1]

A solution of 0.5 M zinc chloride in THF (300 uL, 0.150 mmol) was introduced to a mixture of 4-((1R,2S)-2-((4-(methylsulfonyl)benzyloxy)methyl)cyclopropyl)piperidine-1-carbonitrile (50 mg, 0.14 mmol), N-hydroxyisobutyrimidamide (30 mg, 0.28 mmol) and p-toluenesulfonic acid monohydrate (48 mg, 0.25 mmol) in THF (2 mL). The reaction vessel was fitted with a reflux condenser and refluxed for 3 hrs. The reaction mixture was cooled to rt, diluted with EtOAc (5 mL) and neutralized by the addition of saturated aqueous sodium bicarbonate solution (5 mL). The layers were cut and the aqueous phase extracted with EtOAc (5 mL×2). The combined organic layers were dried over MgSO₄, filtered, and concentrated under reduced pressure. The residue was loaded onto 2×2000 micron silica preparative TLC plates (uv 254 active) which were developed using 50% EtOAc/Hex as the solvent system. The desired silica (Rf=0.3 @ 50% EtOAc/Hex) was collected and extracted to give the title compound (28 mg, 46%). ¹H NMR (300 MHz, CDCl₃) δ 8.93 (d, 2H), 7.53 (d, 2H), 4.60 (s, 2H), 4.15-4.05 (m, 2H), 3.36 (d, 2H), 3.05 (s, 3H), 3.10-2.80 (m, 3H), 1.90-1.75 (m, 2H), 1.50-1.30 (m, 2H), 1.28 (d, 6H), 1.05-0.75 (m, 2H), 0.60-0.40 (m, 3H). LC/MS (m/z): 434 (M+H)⁺, GPR119 Human EC₅₀: 3.8 nM (HTRF assay).

The example in Table 3 was synthesized according to the methods described in Example 21 employing Intermediates 3 and 15 in addition to commercially available starting materials.

TABLE 3

| Example | | Observed | GPR119 |
|---|---|---|---|
| # | Chemical Structure | Mass | Human EC₅₀ |
| 22 | 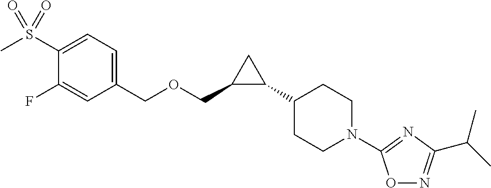 [SMILES: CC(C)c1noc(N2CCC([C@H]3C[C@@H]3COCc3ccc(S(C)(=O)=O)c(F)c3)CC2)n1] | 452 [M + H]⁺ | 3.2 nM HTRF assay |

### Example 23

### Preparation of 1-methylcyclopropyl 4-((1R,2S)-2-((4-(2-(azetidin-1-yl)-2-oxoethyl)-2-fluorobenzyloxy)methyl)cyclopropyl)piperidine-1-carboxylate

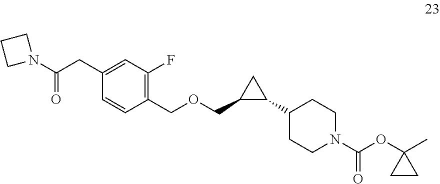 [SMILES: CC1(OC(=O)N2CCC([C@H]3C[C@@H]3COCc3ccc(CC(=O)N4CCC4)cc3F)CC2)CC1]

### Step A: Benzyl 4-((1R,2S)-2-((4-bromo-2-fluorobenzyloxy)methyl)cyclopropyl)piperidine-1-carboxylate

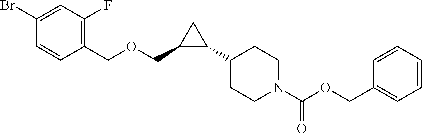 [SMILES: O=C(OCc1ccccc1)N1CCC([C@H]2C[C@@H]2COCc2ccc(Br)cc2F)CC1]

This intermediate was generated by following Step A described in the synthesis of Example 1 using Intermediate 1 and commercially available starting materials. LC/MS (m/z): 476 (M+H)⁺.

### Step B: Benzyl 4-((1R,2S)-2-((4-(2-tert-butoxy-2-oxoethyl)-2-fluorobenzyloxy)methyl)cyclopropyl)piperidine-1-carboxylate

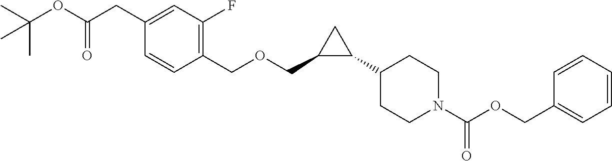 [SMILES: CC(C)(C)OC(=O)Cc1ccc(COC[C@H]2C[C@@H]2C2CCN(C(=O)OCc3ccccc3)CC2)c(F)c1]

A solution of 0.5 M 2-(tert-butyloxy)-2-oxoethylzinc chloride in Et₂O (20 mL, 9.9 mmol) was added to a mixture of benzyl 4-((1R,2S)-2-((4-bromo-2-fluorobenzyloxy)methyl)cyclopropyl)piperidine-1-carboxylate (1.57 g, 3.3 mmol), Pd₂(dba)₃ (150 mg, 0.165 mmol), and X-PHOS (157 mg, 0.33 mmol) in anhydrous THF (3 mL). The reaction mixture was stirred and heated at 65° C. overnight. The following morning the mixture was cooled to rt and filtered through a plug of celite, washing with EtOAc. The volatiles were removed under reduced pressure and the residue was loaded onto a silica column (KP-Sil 50 g SNAP column, Biotage system) eluting with a range of 5-45% EtOAc/Hex over 13 CV to give the desired compound (1.25 g, 74%). LC/MS (m/z): 512 (M+H)⁺.

### Step C: 2-(4-((((1S,2R)-2-(1-(Benzyloxycarbonyl)piperidin-4-yl)cyclopropyl)methoxy)methyl)-3-fluorophenyl)acetic acid

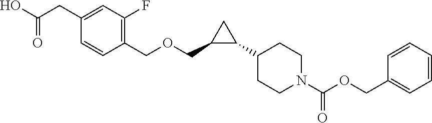 [SMILES: O=C(O)Cc1ccc(COC[C@H]2C[C@@H]2C2CCN(C(=O)OCc3ccccc3)CC2)c(F)c1]

A solution of 4 M HCl in dioxane (3.85 mL, 15.4 mmol) was added to a solution of benzyl 4-((1R,2S)-2-((4-(2-tert-butoxy-2-oxoethyl)-2-fluorobenzyloxy)methyl)cyclopropyl)piperidine-1-carboxylate (788 mg, 1.54 mmol) in DCM (4 mL). This mixture was stirred at 35° C. for 4 hrs. The reaction mixture was subsequently concentrated under reduced pressure to afford the title compound (681 mg, 97%) as a crude product to be used for the next step. LC/MS (m/z): 456 (M+H)⁺.

### Step D: Benzyl 4-((1R,2S)-2-((4-(2-(azetidin-1-yl)-2-oxoethyl)-2-fluorobenzyloxy)methyl)cyclopropyl)piperidine-1-carboxylate

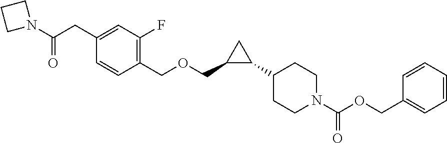 [SMILES: O=C(Cc1ccc(COC[C@H]2C[C@@H]2C2CCN(C(=O)OCc3ccccc3)CC2)c(F)c1)N1CCC1]

A solution of 2-(4-((((1S,2R)-2-(1-(benzyloxycarbonyl)piperidin-4-yl)cyclopropyl)methoxy)methyl)-3-fluorophenyl)acetic acid (127 mg, 0.278 mmol), HOBT.H₂O (64 mg, 0.418 mmol), and EDC.HCl (80 mg, 0.418 mmol) dissolved in DCM (1 mL) was stirred at rt for 30 min. Azetidine (60 uL, 0.861 mmol) was added to this solution and the reaction aged at rt for 3 hrs. The reaction mixture was diluted with DCM (1 mL) and the residue loaded directly onto 2×2000 micron silica preparative TLC plates (uv 254 active) which were developed using 60% EtOAc/Hex as the solvent system. The desired silica (Rf=0.3 @ 60% EtOAc/Hex) was collected and extracted to give the title compound (88 mg, 64%). LC/MS (m/z): 495 (M+H)⁺.

### Step E: 1-(Azetidin-1-yl)-2-(3-fluoro-4-((((1S,2R)-2-(piperidin-4-yl)cyclopropyl)methoxy)methyl)phenyl)ethanone

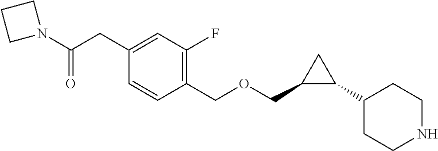 [SMILES: O=C(Cc1ccc(COC[C@H]2C[C@@H]2C2CCNCC2)c(F)c1)N1CCC1]

A solution of benzyl 4-((1R,2S)-2-((4-(2-(azetidin-1-yl)-2-oxoethyl)-2-fluorobenzyloxy)methyl)cyclopropyl)piperidine-1-carboxylate (88 mg, 0.178 mmol) and palladium on carbon (10 mg, 0.095 mmol) in MeOH (10 mL) was stirred under 1 atm of hydrogen at rt for 1 hr. The reaction mixture was filtered through a plug of celite which was washed with MeOH. The volatiles were removed under reduced pressure to afford the title compound (56 mg, 88%) as a crude product to be used for the next step. LC/MS (m/z): 361 (M+H)⁺.

### Step F: 1-Methylcyclopropyl 4-((1R,2S)-2-((4-(2-(azetidin-1-yl)-2-oxoethyl)-2-fluorobenzyloxy)methyl)cyclopropyl)piperidine-1-carboxylate

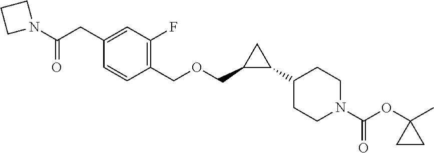 [SMILES: CC1(OC(=O)N2CCC([C@H]3C[C@@H]3COCc3ccc(CC(=O)N4CCC4)cc3F)CC2)CC1]

1-(Azetidin-1-yl)-2-(3-fluoro-4-((((1S,2R)-2-(piperidin-4-yl)cyclopropyl)methoxy)methyl)phenyl)ethanone was subjected to Step B described in the synthesis of Example 14 to give the title compound. ¹H NMR (400 MHz, CDCl₃) δ 7.29 (t, 1H), 6.96 (d, 1H), 6.90 (d, 1H), 4.65-4.51 (m, 4H), 4.09 (t, 2H), 4.00 (t, 2H), 3.39 (s, 2H), 3.26 (dd, 2H), 2.59 (t, 2H), 2.25-2.17 (m, 2H) 1.65 (dd, 2H), 1.48 (s, 3H), 1.18-1.16 (m, 2H), 0.88-0.68 (m, 4H), 0.58-0.52 (m, 2H), 0.45-0.26 (m, 3H). LC/MS (m/z): 459 (M+H)⁺, GPR119 Human EC₅₀: 13 nM (LCMP assay).

### Examples 24-64

### Preparation of 5-chloro-2-(4-((1S,2S)-2-(2-(3-fluoro-4-(methylsulfonyl)phenoxy)ethyl)cyclopropyl)piperidin-1-yl)pyrimidine

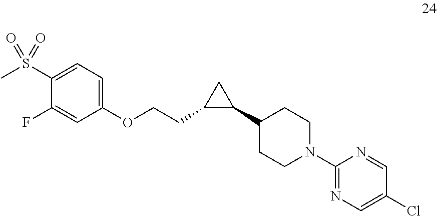 [SMILES: CS(=O)(=O)c1ccc(OCC[C@@H]2C[C@H]2C2CCN(c3ncc(Cl)cn3)CC2)cc1F]

### Step A: Benzyl 4-((1S,2S)-2-(2-(3-fluoro-4-(methylsulfonyl)phenoxy)ethyl)cyclopropyl)piperidine-1-carboxylate

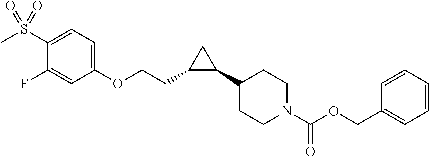 [SMILES: CS(=O)(=O)c1ccc(OCC[C@@H]2C[C@H]2C2CCN(C(=O)OCc3ccccc3)CC2)cc1F]

DIAD (800 mg, 4.0 mmol) was slowly added to a mixture of 3-fluoro-4-(methylsulfonyl)phenol (Intermediate 17) (400 mg, 2.1 mmol), benzyl 4-((1S,2S)-2-(2-hydroxyethyl)cyclopropyl)piperidine-1-carboxylate (Intermediate 5) (600 mg, 2.1 mmol), and triphenylphosphine (1.5 g, 6.0 mmol) in anhydrous THF (50 mL) that had been cooled to 0° C. and placed under an inert atmosphere. The reaction was warmed to rt and aged for 2 hrs. The reaction mixture was concentrated under reduced pressure and the residue was purified by column chromatography (PE: EtOAc=3:1˜1:1) to give the title compound (850 mg, 85%). LC/MS (m/z): 476 (M+H)⁺.

### Step B: 4-((1S,2S)-2-(2-(3-Fluoro-4-(methylsulfonyl)phenoxy)ethyl)cyclopropyl)piperidine

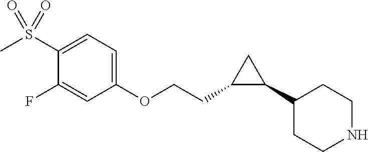 [SMILES: CS(=O)(=O)c1ccc(OCC[C@@H]2C[C@H]2C2CCNCC2)cc1F]

A solution of benzyl 4-((1S,2S)-2-(2-(3-fluoro-4-(methylsulfonyl)phenoxy)ethyl)cyclopropyl)piperidine-1-carboxylate (850 mg, 1.78 mmol) and palladium on carbon (40 mg, 0.384 mmol) in MeOH (100 mL) was stirred under 1 atm of hydrogen at rt for 1 hr. The reaction mixture was filtered through a plug of celite which was washed with MeOH. The volatiles were removed under reduced pressure to afford the title compound (500 mg, 82%) as a crude product to be used for the next step. LC/MS (m/z): 342 (M+H)⁺.

### Step C: 5-Chloro-2-(4-((1S,2S)-2-(2-(3-fluoro-4-(methylsulfonyl)phenoxy)ethyl)cyclopropyl)piperidin-1-yl)pyrimidine

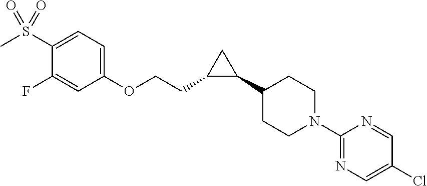 [SMILES: CS(=O)(=O)c1ccc(OCC[C@@H]2C[C@H]2C2CCN(c3ncc(Cl)cn3)CC2)cc1F]

4-((1S,2S)-2-(2-(3-Fluoro-4-(methylsulfonyl)phenoxy)ethyl)cyclopropyl)piperidine (50 mg, 0.22 mmol) was subjected to Step E described in the synthesis of Example 1 to give the title compound. ¹H NMR (500 MHz, CD₃CN) δ 8.18 (s, 2H), 7.84 (t, 1H), 6.80 (d, 1H), 6.72 (d, 1H), 4.65-4.61 (m, 2H), 4.05 (t, 2H), 3.18 (s, 3H), 2.80 (t, 2H), 1.83-1.74 (m, 3H), 1.64-1.57 (m, 1H), 1.32-1.23 (m, 2H), 0.85-0.69 (m, 2H), 0.40-0.30 (m, 3H). LC/MS (m/z): 454 (M+H)⁺, GPR119 Human EC₅₀: 8.3 nM (LCMP assay).

The examples in Table 4 were synthesized according to the methods described in Example 24 employing Intermediates 4, 5, 9, 17-22, 24-26, 29, and 30 in addition to commercially available starting materials.

TABLE 4

| Example | | Observed | GPR119 |
|---|---|---|---|
| # | Chemical Structure | Mass | Human EC₅₀ |
| 25 | 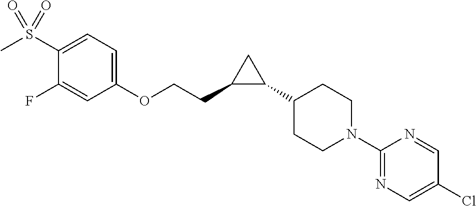 [SMILES: CS(=O)(=O)c1ccc(OCC[C@H]2C[C@@H]2C2CCN(c3ncc(Cl)cn3)CC2)cc1F] | 454 [M + H]⁺ | 4.1 nM LCMP assay |
| 26 | 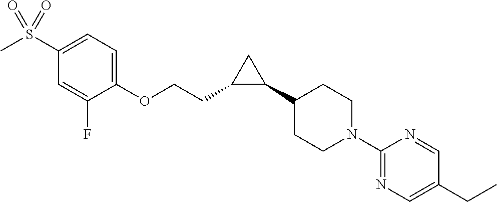 [SMILES: CCc1cnc(N2CCC([C@@H]3C[C@H]3CCOc3ccc(S(C)(=O)=O)cc3F)CC2)nc1] | 448 [M + H]⁺ | 9.4 nM LCMP assay |
| 27 | 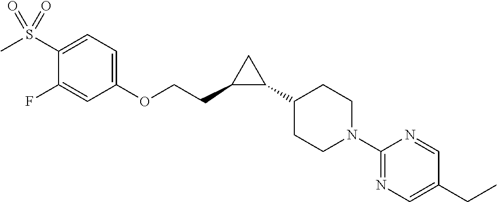 [SMILES: CCc1cnc(N2CCC([C@H]3C[C@@H]3CCOc3ccc(S(C)(=O)=O)c(F)c3)CC2)nc1] | 448 [M + H]⁺ | 5.5 nM LCMP assay |
| 28 | 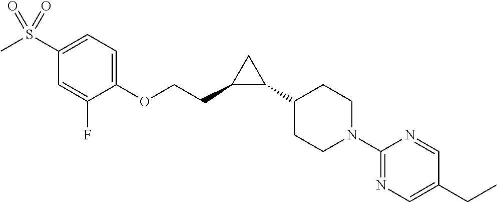 [SMILES: CCc1cnc(N2CCC([C@H]3C[C@@H]3CCOc3ccc(S(C)(=O)=O)cc3F)CC2)nc1] | 448 [M + H]⁺ | 8.8 nM LCMP assay |
| 29 | 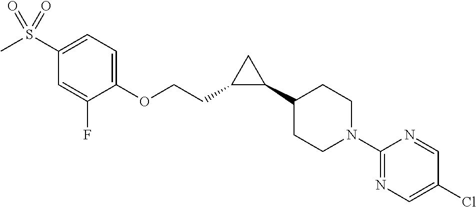 [SMILES: CS(=O)(=O)c1ccc(OCC[C@@H]2C[C@H]2C2CCN(c3ncc(Cl)cn3)CC2)c(F)c1] | 454 [M + H]⁺ | 17 nM LCMP assay |
| 30 | 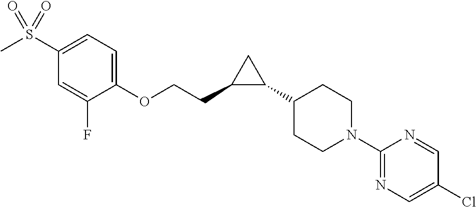 [SMILES: CS(=O)(=O)c1ccc(OCC[C@H]2C[C@@H]2C2CCN(c3ncc(Cl)cn3)CC2)c(F)c1] | 454 [M + H]⁺ | 23 nM LCMP assay |
| 31 | 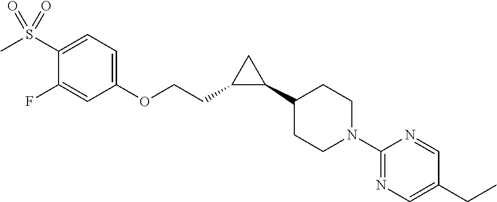 [SMILES: CCc1cnc(N2CCC([C@@H]3C[C@H]3CCOc3ccc(S(C)(=O)=O)c(F)c3)CC2)nc1] | 448 [M + H]⁺ | 16 nM LCMP assay |
| 32 | 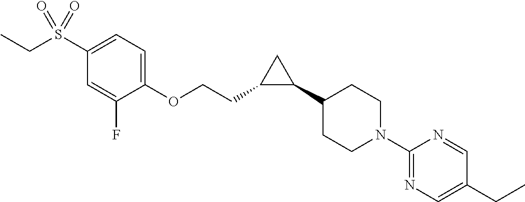 [SMILES: CCc1cnc(N2CCC([C@@H]3C[C@H]3CCOc3ccc(S(=O)(=O)CC)cc3F)CC2)nc1] | 462 [M + H]⁺ | 18 nM LCMP assay |
| 33 | 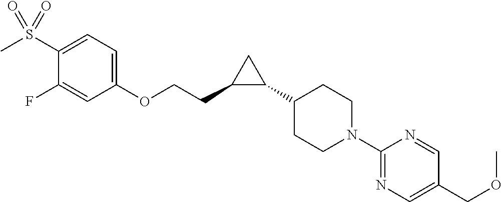 | 464 [M + H]⁺ | 15 nM LCMP assay |
| 34 | 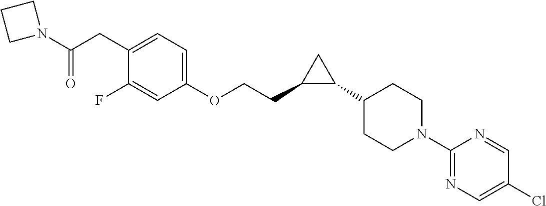 [SMILES: O=C(Cc1ccc(OCC[C@H]2C[C@@H]2C2CCN(c3ncc(Cl)cn3)CC2)cc1F)N1CCC1] | 473 [M + H]⁺ | 0.64 nM LCMP assay |
| 35 | 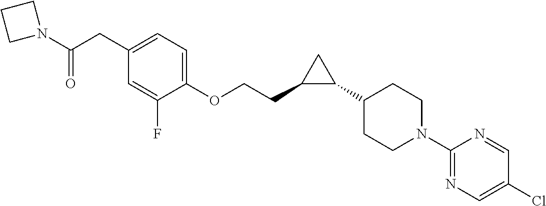 [SMILES: O=C(Cc1ccc(OCC[C@H]2C[C@@H]2C2CCN(c3ncc(Cl)cn3)CC2)c(F)c1)N1CCC1] | 473 [M + H]⁺ | 4.6 nM LCMP assay |
| 36 | 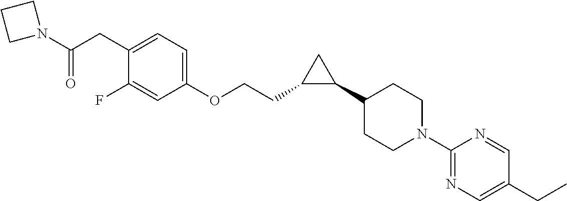 [SMILES: CCc1cnc(N2CCC([C@@H]3C[C@H]3CCOc3ccc(CC(=O)N4CCC4)c(F)c3)CC2)nc1] | 467 [M + H]⁺ | 5.5 nM LCMP assay |
| 37 | 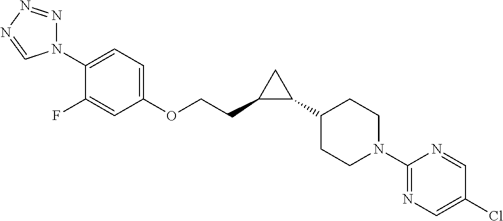 [SMILES: Fc1cc(OCC[C@H]2C[C@@H]2C2CCN(c3ncc(Cl)cn3)CC2)ccc1-n1cnnn1] | 444 [M + H]⁺ | 6.8 nM LCMP assay |
| 38 | 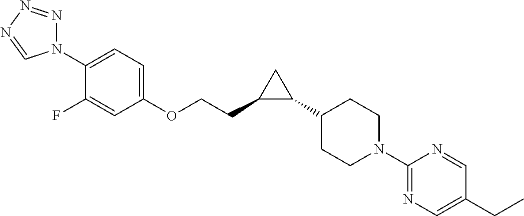 [SMILES: CCc1cnc(N2CCC([C@H]3C[C@@H]3CCOc3ccc(-n4cnnn4)c(F)c3)CC2)nc1] | 438 [M + H]⁺ | 5.3 nM LCMP assay |
| 39 | 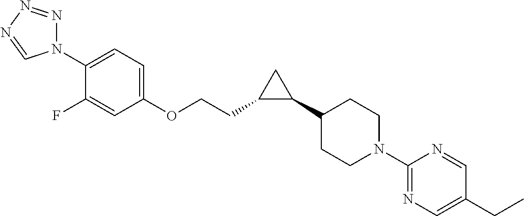 [SMILES: CCc1cnc(N2CCC([C@@H]3C[C@H]3CCOc3ccc(-n4cnnn4)c(F)c3)CC2)nc1] | 438 [M + H]⁺ | 8.7 nM LCMP assay |
| 40 | 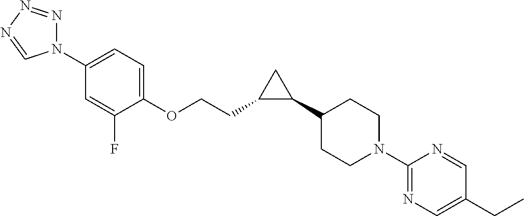 [SMILES: CCc1cnc(N2CCC([C@@H]3C[C@H]3CCOc3ccc(-n4cnnn4)cc3F)CC2)nc1] | 438 [M + H]⁺ | 5.0 nM LCMP assay |
| 41 | 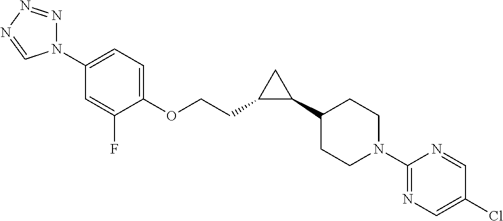 [SMILES: Fc1cc(-n2cnnn2)ccc1OCC[C@@H]1C[C@H]1C1CCN(c2ncc(Cl)cn2)CC1] | 444 [M + H]⁺ | 5.6 nM LCMP assay |
| 42 | 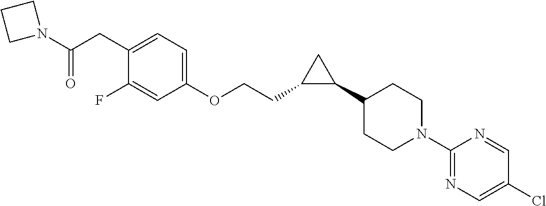 [SMILES: O=C(Cc1ccc(OCC[C@@H]2C[C@H]2C2CCN(c3ncc(Cl)cn3)CC2)cc1F)N1CCC1] | 473 [M + H]⁺ | 1.8 nM LCMP assay |
| 43 | 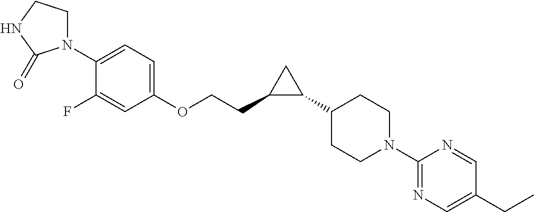 [SMILES: CCc1cnc(N2CCC([C@H]3C[C@@H]3CCOc3ccc(N4CCNC4=O)c(F)c3)CC2)nc1] | 454 [M + H]⁺ | 9.6 nM LCMP assay |
| 44 | 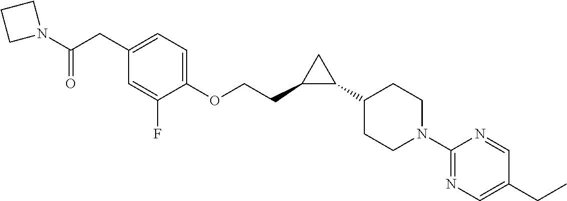 [SMILES: CCc1cnc(N2CCC([C@H]3C[C@@H]3CCOc3ccc(CC(=O)N4CCC4)cc3F)CC2)nc1] | 467 [M + H]⁺ | 2.9 nM LCMP assay |
| 45 | 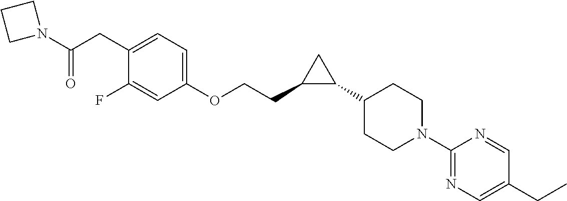 [SMILES: CCc1cnc(N2CCC([C@H]3C[C@@H]3CCOc3ccc(CC(=O)N4CCC4)c(F)c3)CC2)nc1] | 467 [M + H]⁺ | 0.80 nM LCMP assay |
| 46 | 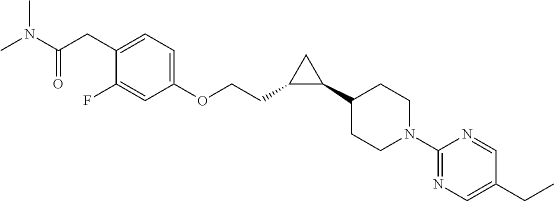 [SMILES: CCc1cnc(N2CCC([C@@H]3C[C@H]3CCOc3ccc(CC(=O)N(C)C)c(F)c3)CC2)nc1] | 455 [M + H]⁺ | 4.4 nM LCMP assay |
| 47 | 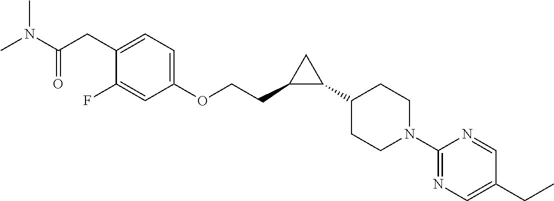 [SMILES: CCc1cnc(N2CCC([C@H]3C[C@@H]3CCOc3ccc(CC(=O)N(C)C)c(F)c3)CC2)nc1] | 455 [M + H]⁺ | 2.9 nM LCMP assay |
| 48 | 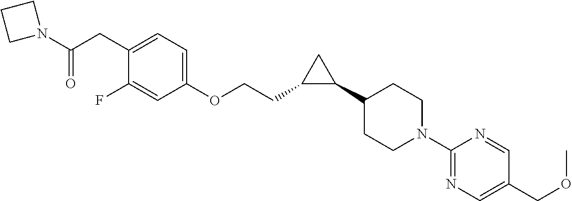 [SMILES: COCc1cnc(N2CCC([C@@H]3C[C@H]3CCOc3ccc(CC(=O)N4CCC4)c(F)c3)CC2)nc1] | 483 [M + H]⁺ | 8.5 nM LCMP assay |
| 49 | 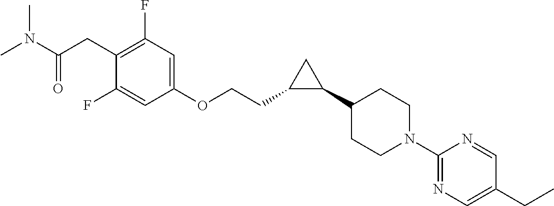 [SMILES: CCc1cnc(N2CCC([C@@H]3C[C@H]3CCOc3cc(F)c(CC(=O)N(C)C)c(F)c3)CC2)nc1] | 473 [M + H]⁺ | 0.70 nM LCMP assay |
| 50 | 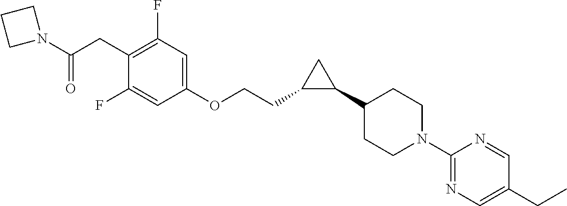 [SMILES: CCc1cnc(N2CCC([C@@H]3C[C@H]3CCOc3cc(F)c(CC(=O)N4CCC4)c(F)c3)CC2)nc1] | 485 [M + H]⁺ | 0.35 nM LCMP assay |
| 51 | 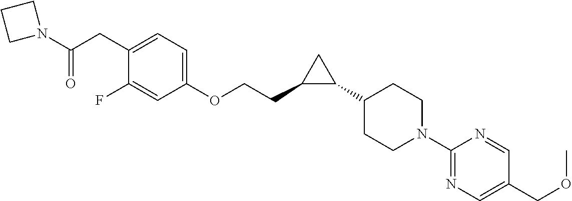 [SMILES: COCc1cnc(N2CCC([C@H]3C[C@@H]3CCOc3ccc(CC(=O)N4CCC4)c(F)c3)CC2)nc1] | 483 [M + H]⁺ | 3.4 nM LCMP assay |
| 52 | 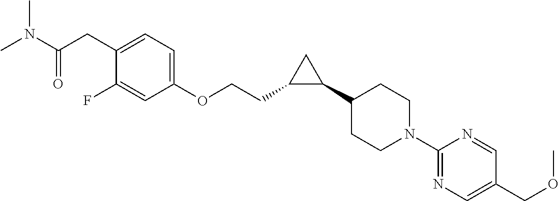 | 471 [M + H]⁺ | 9.6 nM LCMP assay |
| 53 | 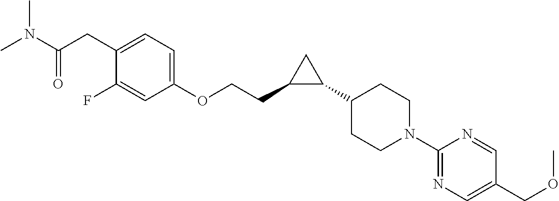 | 471 [M + H]⁺ | 5.9 nM LCMP assay |
| 54 | 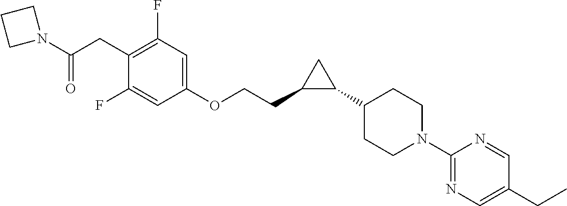 [SMILES: CCc1cnc(N2CCC([C@H]3C[C@@H]3CCOc3cc(F)c(CC(=O)N4CCC4)c(F)c3)CC2)nc1] | 485 [M + H]⁺ | 0.25 nM LCMP assay |
| 55 | 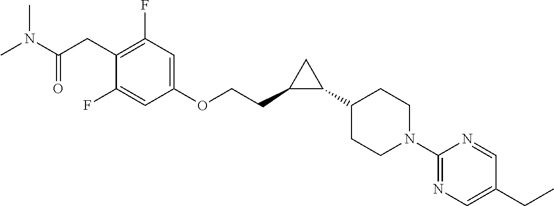 [SMILES: CCc1cnc(N2CCC([C@H]3C[C@@H]3CCOc3cc(F)c(CC(=O)N(C)C)c(F)c3)CC2)nc1] | 473 [M + H]⁺ | 0.34 nM LCMP assay |
| 56 | 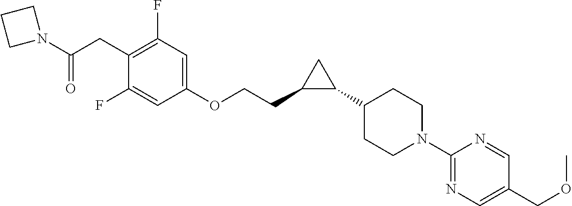 [SMILES: COCc1cnc(N2CCC([C@H]3C[C@@H]3CCOc3cc(F)c(CC(=O)N4CCC4)c(F)c3)CC2)nc1] | 501 [M + H]⁺ | 0.40 nM LCMP assay |
| 57 | 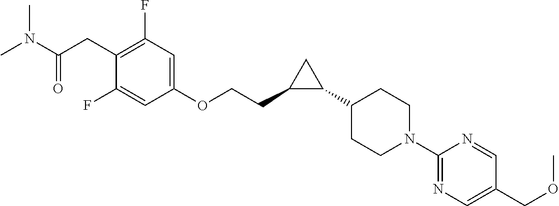 | 489 [M + H]⁺ | 0.63 nM LCMP assay |
| 58 | 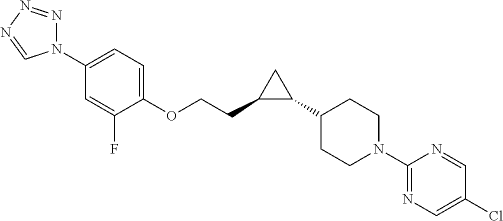 [SMILES: Fc1cc(-n2cnnn2)ccc1OCC[C@H]1C[C@@H]1C1CCN(c2ncc(Cl)cn2)CC1] | 444 [M + H]⁺ | 11 nM LCMP assay |
| 59 | 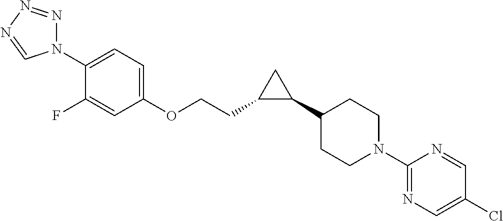 [SMILES: Fc1cc(OCC[C@@H]2C[C@H]2C2CCN(c3ncc(Cl)cn3)CC2)ccc1-n1cnnn1] | 444 [M + H]⁺ | 13 nM LCMP assay |
| 60 | 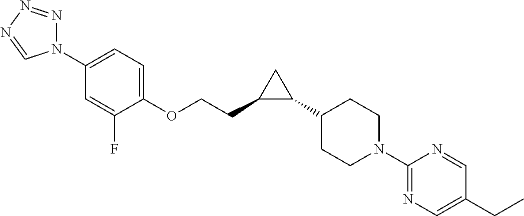 [SMILES: CCc1cnc(N2CCC([C@H]3C[C@@H]3CCOc3ccc(-n4cnnn4)cc3F)CC2)nc1] | 438 [M + H]⁺ | 17 nM LCMP assay |
| 61 | 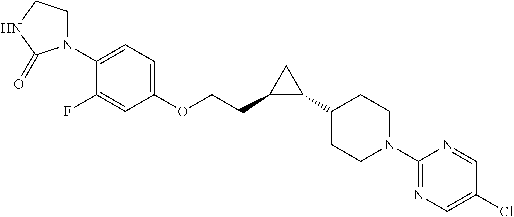 [SMILES: O=C1NCCN1c1ccc(OCC[C@H]2C[C@@H]2C2CCN(c3ncc(Cl)cn3)CC2)cc1F] | 460 [M + H]⁺ | 17 nM LCMP assay |
| 62 | 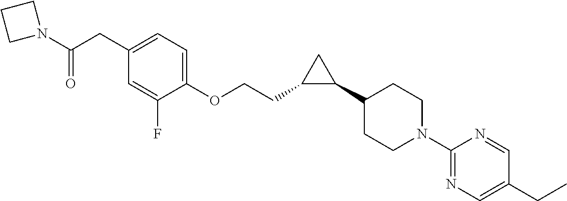 [SMILES: CCc1cnc(N2CCC([C@@H]3C[C@H]3CCOc3ccc(CC(=O)N4CCC4)cc3F)CC2)nc1] | 467 [M + H]⁺ | 11 nM LCMP assay |
| 63 | 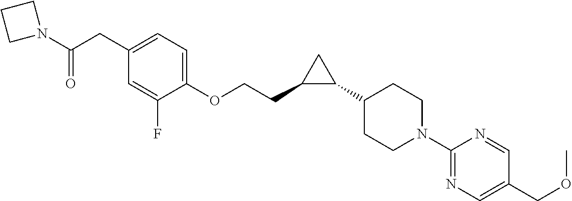 [SMILES: COCc1cnc(N2CCC([C@H]3C[C@@H]3CCOc3ccc(CC(=O)N4CCC4)cc3F)CC2)nc1] | 483 [M + H]⁺ | 15 nM LCMP assay |
| 64 | 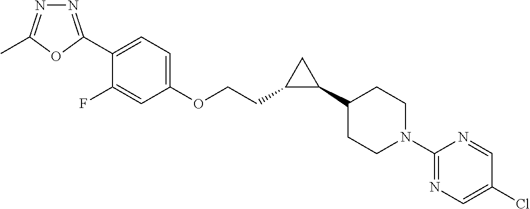 [SMILES: Cc1nnc(-c2ccc(OCC[C@@H]3C[C@H]3C3CCN(c4ncc(Cl)cn4)CC3)cc2F)o1] | 458 [M + H]⁺ | 8.4 nM HTRF assay |

### Examples 65-93

Preparation of 5-(4-((1S,2S)-2-(2-(3-fluoro-4-(methylsulfonyl)phenoxy)ethyl)cyclopropyl)piperidin-1-yl)-3-isopropyl-1,2,4-oxadiazole

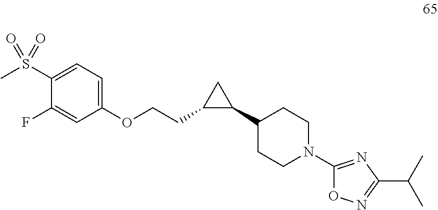 [SMILES: CC(C)c1noc(N2CCC([C@@H]3C[C@H]3CCOc3ccc(S(C)(=O)=O)c(F)c3)CC2)n1]

4-((1S,2S)-2-(2-(2-Fluoro-4-(methylsulfonyl)phenoxy)ethyl)cyclopropyl)piperidine (the product of Step B in the synthesis of Example 24) was subjected to Steps B-C described in the synthesis of Example 21 to give the title compound. ¹H NMR (500 MHz, CD₃CN) δ 7.71-7.64 (q, 2H), 7.10-7.06 (q, 1H), 4.16-4.10 (m, 4H), 3.06 (s, 3H), 3.00-2.93 (m, 2H), 2.89-2.85 (m, 1H), 1.86-1.70 (m, 3H), 1.70-1.63 (m, 1H), 1.43-1.37 (m, 2H), 1.29-1.25 (m, 6H), 0.80-0.69 (m, 2H), 0.48-0.30 (m, 3H). LC/MS (m/z): 452 (M+H)⁺, GPR119 Human EC₅₀: 7.7 nM (LCMP assay).

The examples in Table 5 were synthesized according to the methods described in Example 65 employing Intermediates 4, 5, 17-20, 22, 24-26, 29, and 30 in addition to commercially available starting materials.

TABLE 5

| Example | | Observed | GPR119 |
|---|---|---|---|
| # | Chemical Structure | Mass | Human EC₅₀ |
| 66 | 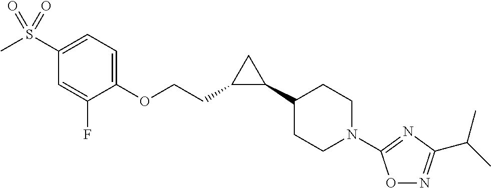 [SMILES: CC(C)c1noc(N2CCC([C@@H]3C[C@H]3CCOc3ccc(S(C)(=O)=O)cc3F)CC2)n1] | 452 [M + H]⁺ | 3.5 nM LCMP assay |
| 67 | 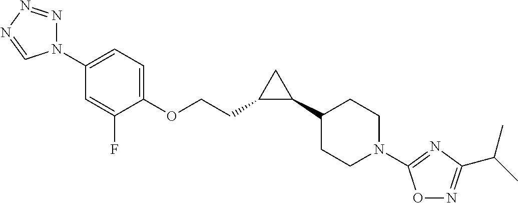 [SMILES: CC(C)c1noc(N2CCC([C@@H]3C[C@H]3CCOc3ccc(-n4cnnn4)cc3F)CC2)n1] | 442 [M + H]⁺ | 6.2 nM LCMP assay |
| 68 | 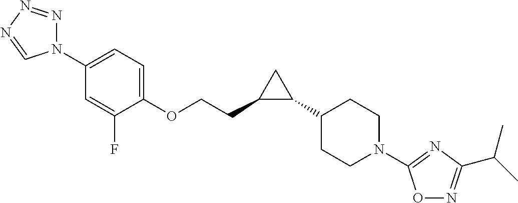 [SMILES: CC(C)c1noc(N2CCC([C@H]3C[C@@H]3CCOc3ccc(-n4cnnn4)cc3F)CC2)n1] | 442 [M + H]⁺ | 3.5 nM LCMP assay |
| 69 | 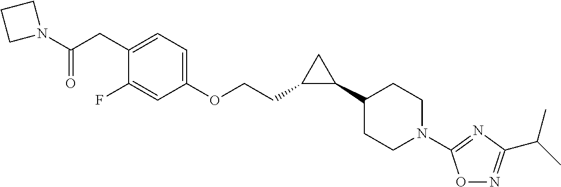 [SMILES: CC(C)c1noc(N2CCC([C@@H]3C[C@H]3CCOc3ccc(CC(=O)N4CCC4)c(F)c3)CC2)n1] | 470 [M + H]⁺ | 2.2 nM LCMP assay |
| 70 | 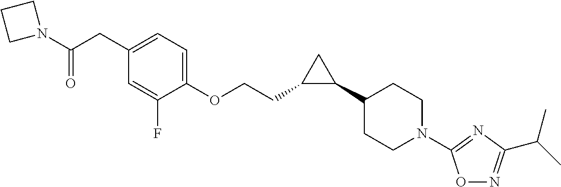 [SMILES: CC(C)c1noc(N2CCC([C@@H]3C[C@H]3CCOc3ccc(CC(=O)N4CCC4)cc3F)CC2)n1] | 471 [M + H]⁺ | 6.8 nM LCMP assay |
| 71 | 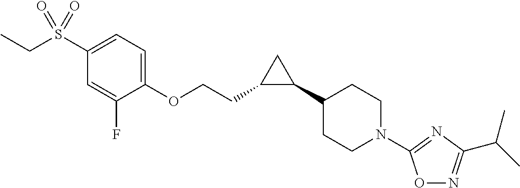 [SMILES: CCS(=O)(=O)c1ccc(OCC[C@@H]2C[C@H]2C2CCN(c3nc(C(C)C)no3)CC2)c(F)c1] | 466 [M + H]⁺ | 3.8 nM LCMP assay |
| 72 | 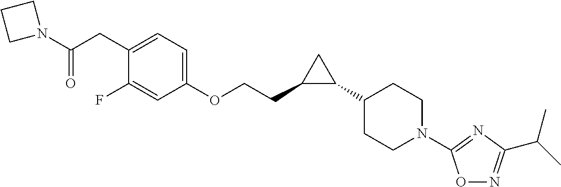 [SMILES: CC(C)c1noc(N2CCC([C@H]3C[C@@H]3CCOc3ccc(CC(=O)N4CCC4)c(F)c3)CC2)n1] | 471 [M + H]⁺ | 2.6 nM LCMP assay |
| 73 | 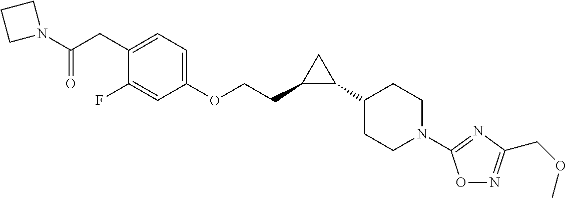 [SMILES: COCc1noc(N2CCC([C@H]3C[C@@H]3CCOc3ccc(CC(=O)N4CCC4)c(F)c3)CC2)n1] | 473 [M + H]⁺ | 6.4 nM LCMP assay |
| 74 | 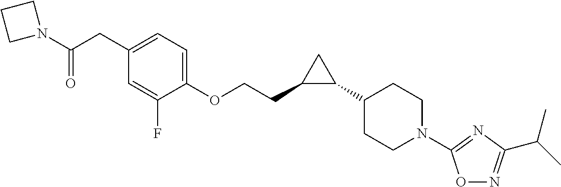 [SMILES: CC(C)c1noc(N2CCC([C@H]3C[C@@H]3CCOc3ccc(CC(=O)N4CCC4)cc3F)CC2)n1] | 471 [M + H]⁺ | 9.5 nM LCMP assay |
| 75 | 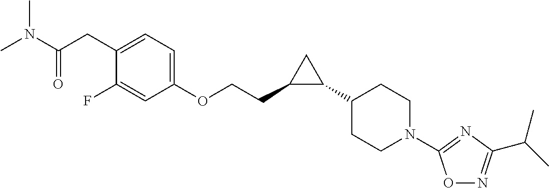 [SMILES: CC(C)c1noc(N2CCC([C@H]3C[C@@H]3CCOc3ccc(CC(=O)N(C)C)c(F)c3)CC2)n1] | 459 [M + H]⁺ | 8.1 nM LCMP assay |
| 76 | 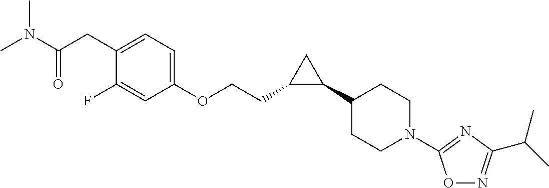 [SMILES: CC(C)c1noc(N2CCC([C@@H]3C[C@H]3CCOc3ccc(CC(=O)N(C)C)c(F)c3)CC2)n1] | 459 [M + H]⁺ | 6.3 nM LCMP assay |
| 77 | 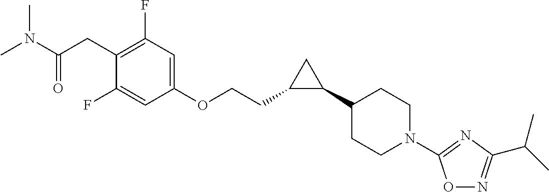 [SMILES: CC(C)c1noc(N2CCC([C@@H]3C[C@H]3CCOc3cc(F)c(CC(=O)N(C)C)c(F)c3)CC2)n1] | 477 [M + H]⁺ | 0.79 nM LCMP assay |
| 78 | 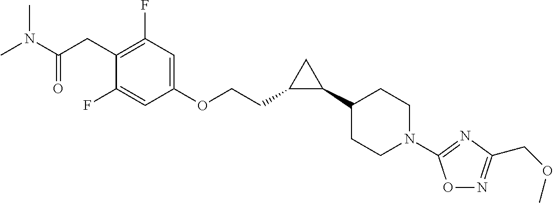 [SMILES: COCc1noc(N2CCC([C@@H]3C[C@H]3CCOc3cc(F)c(CC(=O)N(C)C)c(F)c3)CC2)n1] | 479 [M + H]⁺ | 6.7 nM LCMP assay |
| 79 | 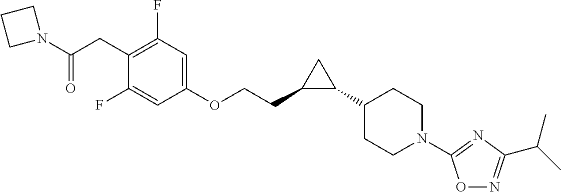 [SMILES: CC(C)c1noc(N2CCC([C@H]3C[C@@H]3CCOc3cc(F)c(CC(=O)N4CCC4)c(F)c3)CC2)n1] | 489 [M + H]⁺ | 0.55 nM LCMP assay |
| 80 | 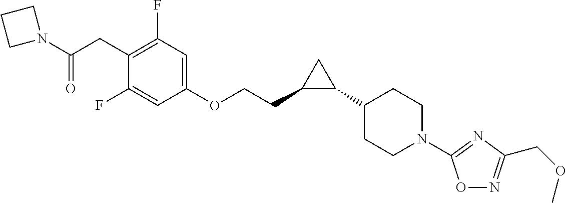 [SMILES: COCc1noc(N2CCC([C@H]3C[C@@H]3CCOc3cc(F)c(CC(=O)N4CCC4)c(F)c3)CC2)n1] | 491 [M + H]⁺ | 2.4 nM LCMP assay |
| 81 | 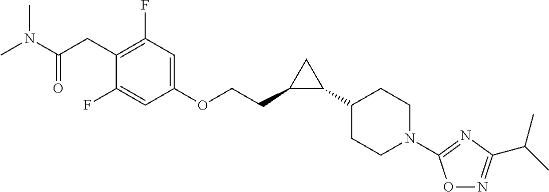 [SMILES: CC(C)c1noc(N2CCC([C@H]3C[C@@H]3CCOc3cc(F)c(CC(=O)N(C)C)c(F)c3)CC2)n1] | 477 [M + H]⁺ | 0.67 nM LCMP assay |
| 82 | 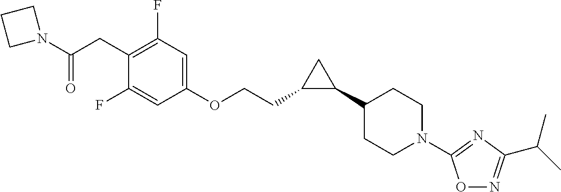 [SMILES: CC(C)c1noc(N2CCC([C@@H]3C[C@H]3CCOc3cc(F)c(CC(=O)N4CCC4)c(F)c3)CC2)n1] | 489 [M + H]⁺ | 0.51 nM LCMP assay |
| 83 | 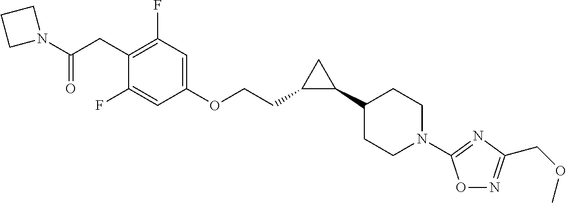 [SMILES: COCc1noc(N2CCC([C@@H]3C[C@H]3CCOc3cc(F)c(CC(=O)N4CCC4)c(F)c3)CC2)n1] | 491 [M + H]⁺ | 5.2 nM LCMP assay |
| 84 | 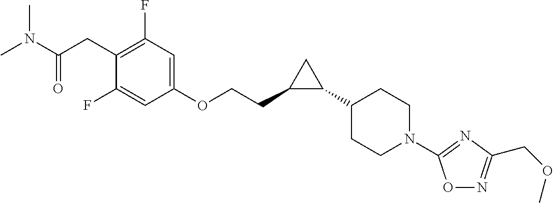 [SMILES: COCc1noc(N2CCC([C@H]3C[C@@H]3CCOc3cc(F)c(CC(=O)N(C)C)c(F)c3)CC2)n1] | 479 [M + H]⁺ | 3.0 nM LCMP assay |
| 85 | 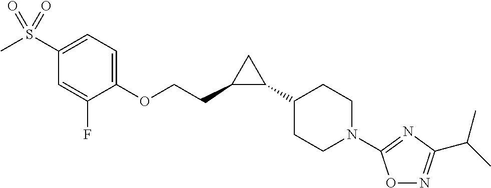 [SMILES: CC(C)c1noc(N2CCC([C@H]3C[C@@H]3CCOc3ccc(S(C)(=O)=O)cc3F)CC2)n1] | 452 [M + H]⁺ | 17 nM LCMP assay |
| 86 | 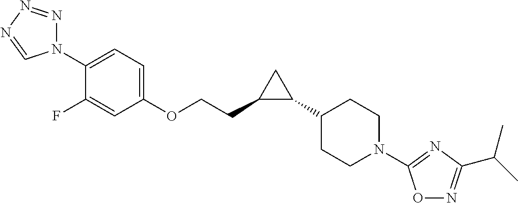 [SMILES: CC(C)c1noc(N2CCC([C@H]3C[C@@H]3CCOc3ccc(-n4cnnn4)c(F)c3)CC2)n1] | 442 [M + H]⁺ | 13 nM LCMP assay |
| 87 | 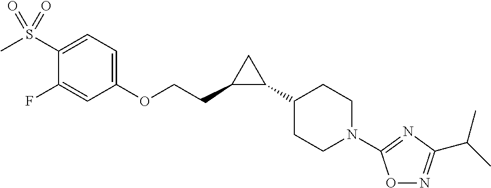 [SMILES: CC(C)c1noc(N2CCC([C@H]3C[C@@H]3CCOc3ccc(S(C)(=O)=O)c(F)c3)CC2)n1] | 452 [M + H]⁺ | 11 nM LCMP assay |
| 88 | 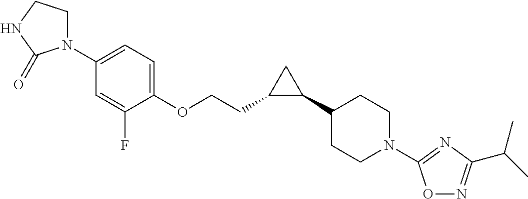 [SMILES: CC(C)c1noc(N2CCC([C@@H]3C[C@H]3CCOc3ccc(N4CCNC4=O)cc3F)CC2)n1] | 458 [M + H]⁺ | 18 nM LCMP assay |
| 89 | 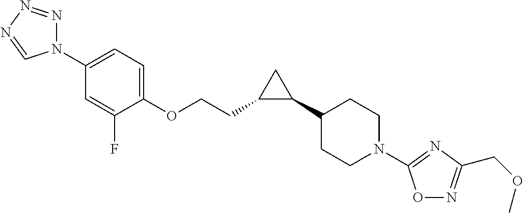 [SMILES: COCc1noc(N2CCC([C@@H]3C[C@H]3CCOc3ccc(-n4cnnn4)cc3F)CC2)n1] | 444 [M + H]⁺ | 23 nM LCMP assay |
| 90 | 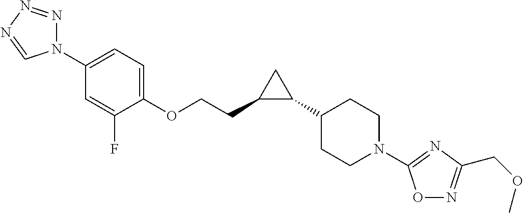 [SMILES: COCc1noc(N2CCC([C@H]3C[C@@H]3CCOc3ccc(-n4cnnn4)cc3F)CC2)n1] | 444 [M + H]⁺ | 20 nM LCMP assay |
| 91 | 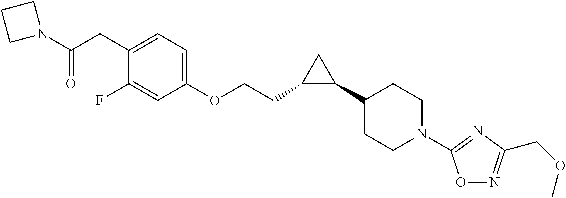 [SMILES: COCc1noc(N2CCC([C@@H]3C[C@H]3CCOc3ccc(CC(=O)N4CCC4)c(F)c3)CC2)n1] | 473 [M + H]⁺ | 19 nM LCMP assay |
| 92 | 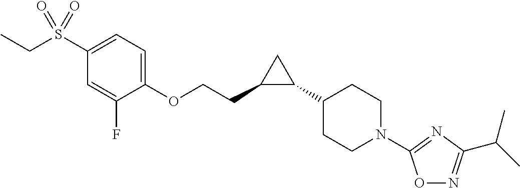 [SMILES: CCS(=O)(=O)c1ccc(OCC[C@H]2C[C@@H]2C2CCN(c3nc(C(C)C)no3)CC2)c(F)c1] | 466 [M + H]⁺ | 13 nM LCMP assay |
| 93 | 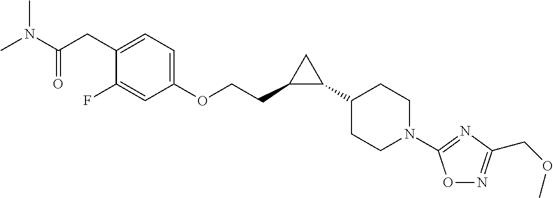 [SMILES: COCc1noc(N2CCC([C@H]3C[C@@H]3CCOc3ccc(CC(=O)N(C)C)c(F)c3)CC2)n1] | 461 [M + H]⁺ | 18 nM LCMP assay |

### Examples 94-108

### Preparation of 1-methylcyclopropyl 4-((1S,2S)-2-(2-(3-fluoro-4-(methylsulfonyl)phenoxy)ethyl)cyclopropyl)piperidine-1-carboxylate

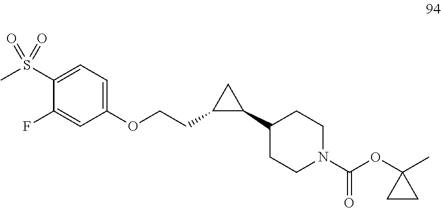

4-((1S,2S)-2-(2-(3-Fluoro-4-(methylsulfonyl)phenoxy)ethyl)cyclopropyl)piperidine (the product of Step B in the synthesis of Example 24) was subjected to Step B described in the synthesis of Example 14 to give the title compound. ¹H NMR (400 MHz, CDCl₃) δ 7.83 (t, 1H), 6.80 (d, 1H), 6.72 (d, 1H), 4.09-3.94 (m, 4H), 3.17 (s, 3H), 2.64 (t, 2H), 1.81-1.58 (m, 4H), 1.52 (s, 3H), 1.22 (br, 2H), 0.84 (m, 2H) 0.70-0.61 (m, 4H), 0.40-0.38 (m, 3H). LC/MS (m/z): 440 (M+H)⁺, GPR119 Human EC₅₀: 23 nM (LCMP assay).

The examples in Table 6 were synthesized according to the methods described in Example 94 employing Intermediates 4, 5, 19, 20, 24-26, 29, and 30 in addition to commercially available starting materials.

TABLE 6

| Example | | Observed | GPR119 |
|---|---|---|---|
| # | Chemical Structure | Mass | Human EC₅₀ |
| 95 | 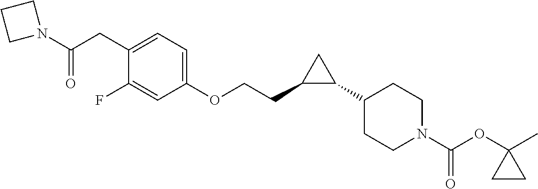 [SMILES: CC1(OC(=O)N2CCC([C@H]3C[C@@H]3CCOc3ccc(CC(=O)N4CCC4)c(F)c3)CC2)CC1] | 459 [M + H]⁺ | 2.6 nM LCMP assay |
| 96 | 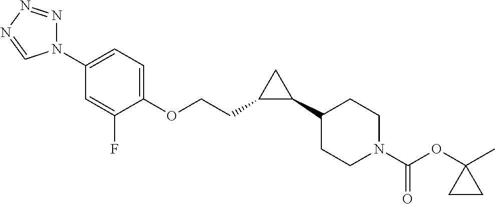 [SMILES: CC1(OC(=O)N2CCC([C@@H]3C[C@H]3CCOc3ccc(-n4cnnn4)cc3F)CC2)CC1] | 430 [M + H]⁺ | 8.2 nM LCMP assay |
| 97 | 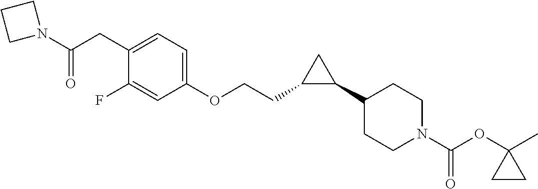 [SMILES: CC1(OC(=O)N2CCC([C@@H]3C[C@H]3CCOc3ccc(CC(=O)N4CCC4)c(F)c3)CC2)CC1] | 459 [M + H]⁺ | 4.2 nM LCMP assay |
| 98 | 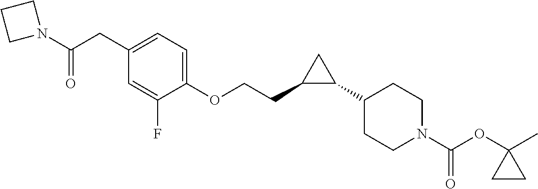 [SMILES: CC1(OC(=O)N2CCC([C@H]3C[C@@H]3CCOc3ccc(CC(=O)N4CCC4)cc3F)CC2)CC1] | 459 [M + H]⁺ | 6.3 nM LCMP assay |
| 99 | 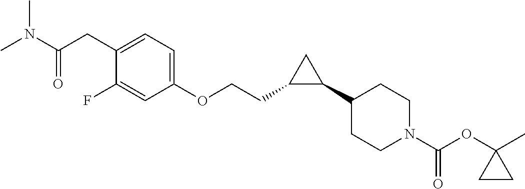 | 447 [M + H]⁺ | 8.8 nM LCMP assay |
| 100 | 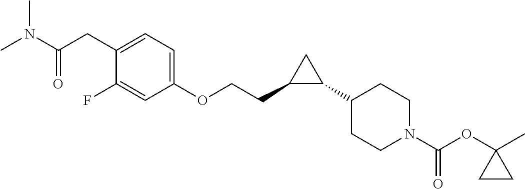 | 447 [M + H]⁺ | 5.5 nM LCMP assay |
| 101 | 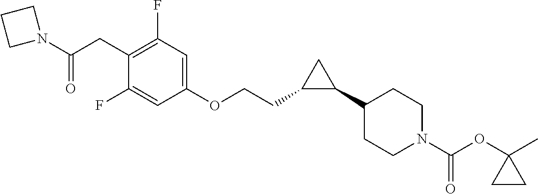 [SMILES: CC1(OC(=O)N2CCC([C@@H]3C[C@H]3CCOc3cc(F)c(CC(=O)N4CCC4)c(F)c3)CC2)CC1] | 477 [M + H]⁺ | 2.0 nM LCMP assay |
| 102 | 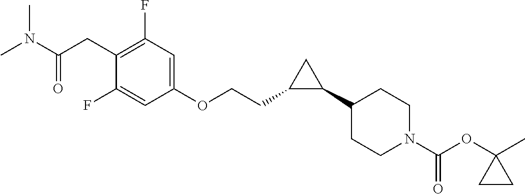 | 465 [M + H]⁺ | 2.1 nM LCMP assay |
| 103 | 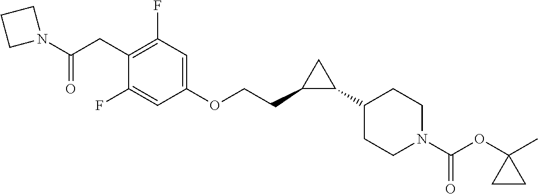 [SMILES: CC1(OC(=O)N2CCC([C@H]3C[C@@H]3CCOc3cc(F)c(CC(=O)N4CCC4)c(F)c3)CC2)CC1] | 477 [M + H]⁺ | 0.70 nM LCMP assay |
| 104 | 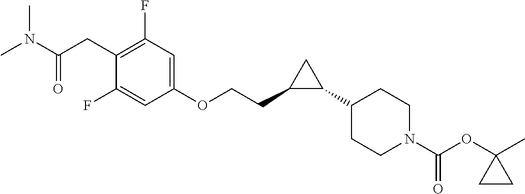 | 465 [M + H]⁺ | 0.69 nM LCMP assay |
| 105 | 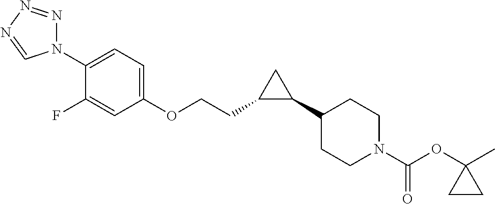 [SMILES: CC1(OC(=O)N2CCC([C@@H]3C[C@H]3CCOc3ccc(-n4cnnn4)c(F)c3)CC2)CC1] | 430 [M + H]⁺ | 15 nM LCMP assay |
| 106 | 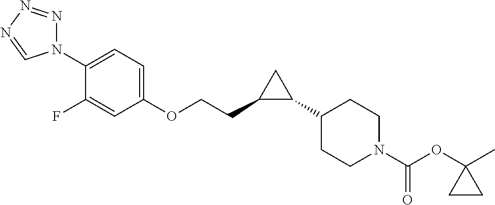 [SMILES: CC1(OC(=O)N2CCC([C@H]3C[C@@H]3CCOc3ccc(-n4cnnn4)c(F)c3)CC2)CC1] | 430 [M + H]⁺ | 14 nM LCMP assay |
| 107 | 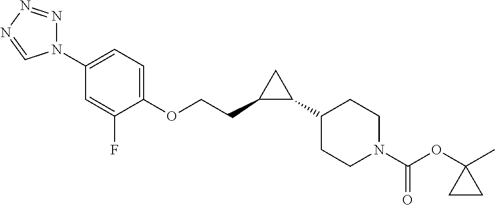 [SMILES: CC1(OC(=O)N2CCC([C@H]3C[C@@H]3CCOc3ccc(-n4cnnn4)cc3F)CC2)CC1] | 430 [M + H]⁺ | 17 nM LCMP assay |
| 108 | 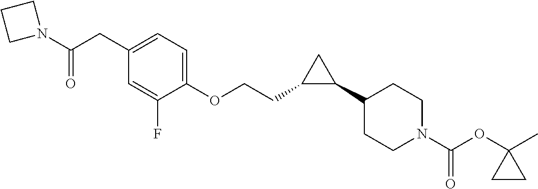 [SMILES: CC1(OC(=O)N2CCC([C@@H]3C[C@H]3CCOc3ccc(CC(=O)N4CCC4)cc3F)CC2)CC1] | 459 [M + H]⁺ | 24 nM LCMP assay |

### Example 109

### Preparation of 5-chloro-2-(4-((1R,2R)-2-(2-(5-(methylsulfonyl)pyridin-2-yloxy)ethyl)cyclopropyl)piperidin-1-yl)pyrimidine

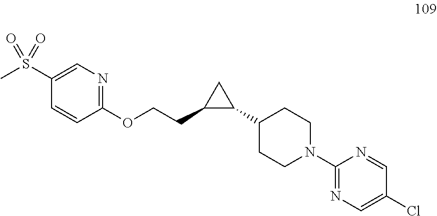 [SMILES: CS(=O)(=O)c1ccc(OCC[C@H]2C[C@@H]2C2CCN(c3ncc(Cl)cn3)CC2)nc1]

### Step A: tert-Butyl 4-((1R,2R)-2-(2-(5-(methylsulfonyl)pyridin-2-yloxy)ethyl)cyclopropyl)piperidine-1-carboxylate

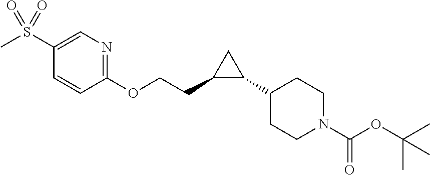 [SMILES: CC(C)(C)OC(=O)N1CCC([C@H]2C[C@@H]2CCOc2ccc(S(C)(=O)=O)cn2)CC1]

Sodium t-butoxide (570 mg, 5.94 mmol) was added to a solution of tert-butyl 4-((1R,2R)-2-(2-hydroxyethyl)cyclopropyl)piperidine-1-carboxylate (Intermediate 6) (600 mg, 1.98 mmol) and 2-bromo-5-(methylsulfonyl)pyridine (558 mg, 2.38 mmol) in anhydrous THF (15 mL) that had been placed under an inert atmosphere at rt. The mixture was stirred and heated at 40° C. for 18 hrs. The solution was cooled to rt and concentrated under reduced pressure. The resulting crude was taken up in EtOAc (30 mL) and washed with water (15 mL×1). The organic phase was dried over MgSO₄, filtered, and concentrated under reduced pressure to afford the title compound as a crude product to be used for the next step. LC/MS (m/z): 425 (M+H)⁺.

### Step B: 5-(Methylsulfonyl)-2-(2-((1R,2R)-2-(piperidin-4-yl)cyclopropyl)ethoxy)pyridine hydrochloride

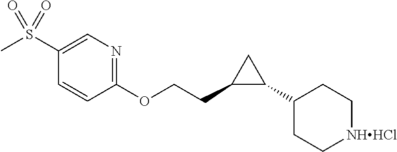

A solution of 4 M HCl in dioxane (3.3 mL, 13.2 mmol) was added to a solution of tert-butyl 4-((1R,2R)-2-(2-(5-(methylsulfonyl)pyridin-2-yloxy)ethyl)cyclopropyl)piperidine-1-carboxylate (560 mg, 1.32 mmol) in DCM (3 mL). This mixture was stirred at rt for 1 hr. The reaction mixture was subsequently concentrated under reduced pressure to afford the title compound (412 mg, 86%) as a crude product to be used for the next step. LC/MS (m/z): 325 (M+H)⁺.

### Step C: 5-Chloro-2-(4-((1R,2R)-2-(2-(5-(methylsulfonyl)pyridin-2-yloxy)ethyl)cyclopropyl)piperidin-1-yl)pyrimidine

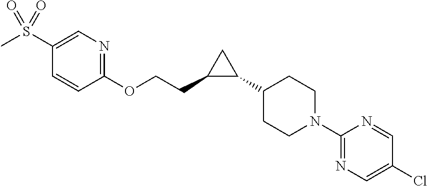 [SMILES: CS(=O)(=O)c1ccc(OCC[C@H]2C[C@@H]2C2CCN(c3ncc(Cl)cn3)CC2)nc1]

5-(Methylsulfonyl)-2-(2-((1R,2R)-2-(piperidin-4-yl)cyclopropyl)ethoxy)pyridine hydrochloride was subjected to Step E described in the synthesis of Example 1 to give the title compound. ¹H NMR (400 MHz, CDCl₃) δ 8.71 (d, 1H), 8.19 (s, 2H), 8.02 (dd, 1H), 6.84 (d, 1H), 4.63 (br, 2H), 4.43 (t, 2H), 3.08 (s, 3H), 2.85-2.78 (m, 2H), 1.84-1.78 (m, 3H), 1.55 (s, 1H), 1.33-1.25 (m, 2H), 0.85-0.78 (m, 1H), 0.68-0.66 (m, 1H), 0.41-0.39 (m, 2H), 0.38-0.37 (m, 1H). LC/MS (m/z): 437 (M+H)⁺, GPR119 Human EC₅₀: 22 nM (LCMP assay).

### Examples 110-114

### Preparation of 3-isopropyl-5-(4-((1R,2R)-2-(2-(5-(methylsulfonyl)pyridin-2-yloxy)ethyl)cyclopropyl)piperidin-1-yl)-1,2,4-oxadiazole

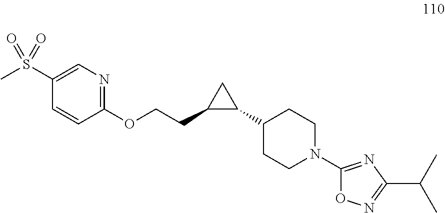 [SMILES: CC(C)c1noc(N2CCC([C@H]3C[C@@H]3CCOc3ccc(S(C)(=O)=O)cn3)CC2)n1]

### Step A: Benzyl 4-((1R,2R)-2-(2-(5-(methylsulfonyl)pyridin-2-yloxy)ethyl)cyclopropyl)piperidine-1-carboxylate

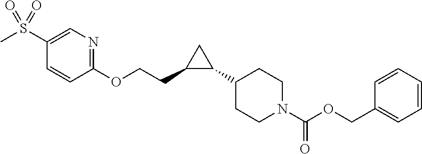 [SMILES: CS(=O)(=O)c1ccc(OCC[C@H]2C[C@@H]2C2CCN(C(=O)OCc3ccccc3)CC2)nc1]

Sodium t-butoxide (330 mg, 5.94 mmol) was added to a solution of benzyl 4-((1R,2R)-2-(2-hydroxyethyl)cyclopropyl)piperidine-1-carboxylate (Intermediate 4) (470 mg, 1.56 mmol) and 2-bromo-5-(methylsulfonyl)pyridine (411 mg, 1.75 mmol) in anhydrous THF (6 mL) that had been placed under an inert atmosphere at rt. The mixture was stirred and heated at 40° C. for 18 hrs. The solution was cooled to rt and concentrated under reduced pressure. The resulting crude was taken up in EtOAc (20 mL) and washed with water (15 mL×1). The organic phase was dried over MgSO₄, filtered, and concentrated under reduced pressure to afford the title compound as a crude product to be used for the next step. LC/MS (m/z): 459 (M+H)⁺.

### Step B: 5-(Methylsulfonyl)-2-(2-((1R,2R)-2-(piperidin-4-yl)cyclopropyl)ethoxy)pyridine

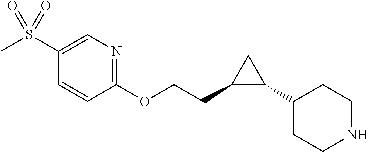 [SMILES: CS(=O)(=O)c1ccc(OCC[C@H]2C[C@@H]2C2CCNCC2)nc1]

A suspension of benzyl 4-((1R,2R)-2-(2-(5-(methylsulfonyl)pyridin-2-yloxy)ethyl)cyclopropyl)piperidine-1-carboxylate (543 mg, 1.19 mmol) and palladium on carbon (28 mg, 0.26 mmol) in MeOH (40 mL) was stirred under 1 atm of hydrogen at rt for 1 hr. The reaction mixture was filtered through a plug of celite which was washed with MeOH. The volatiles were removed under reduced pressure to afford the title compound (333 mg, 86%) as a crude product to be used for the next step. LC/MS (m/z): 325 (M+H)⁺.

### Step C: 3-Isopropyl-5-(4-((1R,2R)-2-(2-(5-(methylsulfonyl)pyridin-2-yloxy)ethyl)cyclopropyl)piperidin-1-yl)-1,2,4-oxadiazole

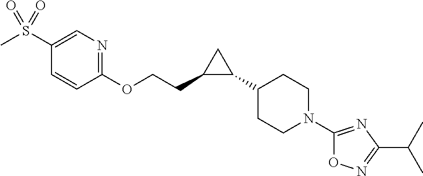 [SMILES: CC(C)c1noc(N2CCC([C@H]3C[C@@H]3CCOc3ccc(S(C)(=O)=O)cn3)CC2)n1]

5-(Methylsulfonyl)-2-(2-((1R,2R)-2-(piperidin-4-yl)cyclopropyl)ethoxy)pyridine was subjected to Steps B-C described in the synthesis of Example 21. ¹H NMR (400 MHz, CDCl₃) δ 8.70 (d, 1H), 8.01 (dd, 1H), 6.82 (d, 1H), 4.41 (t, 2H), 4.07 (s, 2H), 3.06 (s, 3H), 3.00-2.94 (m, 2H), 2.90-2.83 (m, 1H), 1.82-1.60 (m, 3H), 1.59-1.55 (m, 1H), 1.39-1.36 (m, 2H), 1.26 (d, 6H), 0.80-0.77 (m, 1H), 0.66-0.65 (m, 1H), 0.43-0.42 (m, 3H). LC/MS (m/z): 435 (M+H)⁺, GPR119 Human EC₅₀: 18 nM (LCMP assay).

The examples in Table 7 were synthesized according to the methods described in Example 110 employing Intermediate 5 and commercially available starting materials.

TABLE 7

| Example | | Observed | GPR119 |
|---|---|---|---|
| # | Chemical Structure | Mass | Human EC₅₀ |
| 111 | 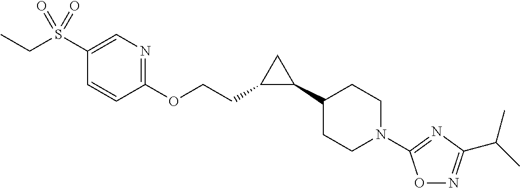 [SMILES: CCS(=O)(=O)c1ccc(OCC[C@@H]2C[C@H]2C2CCN(c3nc(C(C)C)no3)CC2)nc1] | 450 [M + H]⁺ | 17 nM LCMP assay |
| 112 | 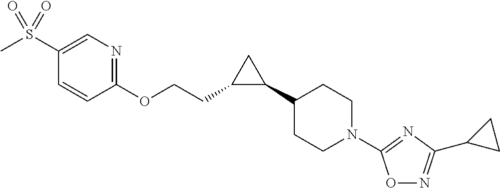 [SMILES: CS(=O)(=O)c1ccc(OCC[C@@H]2C[C@H]2C2CCN(c3nc(C4CC4)no3)CC2)nc1] | 433 [M + H]⁺ | 6.1 nM HTRF assay |
| 113 | 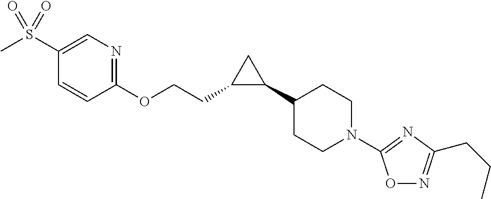 [SMILES: CCCc1noc(N2CCC([C@@H]3C[C@H]3CCOc3ccc(S(C)(=O)=O)cn3)CC2)n1] | 435 [M + H]⁺ | 7.9 nM HTRF assay |
| 114 | 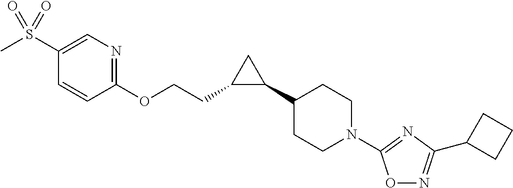 [SMILES: CS(=O)(=O)c1ccc(OCC[C@@H]2C[C@H]2C2CCN(c3nc(C4CCC4)no3)CC2)nc1] | 447 [M + H]⁺ | 4.8 nM HTRF assay |

### Examples 115-116

### Preparation of 1-(azetidin-1-yl)-2-(6-(2-((1R,2R)-2-(1-(5-ethylpyrimidin-2-yl)piperidin-4-yl)cyclopropyl)ethoxy)pyridin-3-yl)ethanone

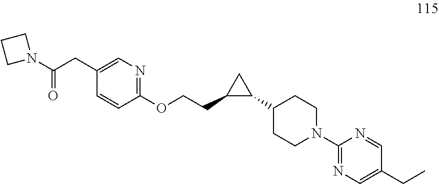 [SMILES: CCc1cnc(N2CCC([C@H]3C[C@@H]3CCOc3ccc(CC(=O)N4CCC4)cn3)CC2)nc1]

### Step A: Benzyl 4-((1R,2R)-2-(2-(5-(2-(azetidin-1-yl)-2-oxoethyl)pyridin-2-yloxy)ethyl)cyclopropyl)piperidine-1-carboxylate

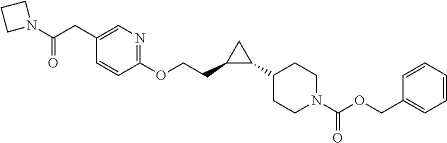 [SMILES: O=C(Cc1ccc(OCC[C@H]2C[C@@H]2C2CCN(C(=O)OCc3ccccc3)CC2)nc1)N1CCC1]

Sodium t-butoxide (144 mg, 1.5 mmol) was added to a solution of benzyl 4-((1R,2R)-2-(2-hydroxyethyl)cyclopropyl)piperidine-1-carboxylate (Intermediate 4) (455 mg, 1.5 mmol) and 1-(azetidin-1-yl)-2-(6-chloropyridin-3-yl)ethanone (Intermediate 23) (400 mg, 1.9 mmol) in anhydrous THF (5 mL) that had been placed under an inert atmosphere at rt. The mixture was irradiated in the microwave at 120° C. for 1 hr. The solution was cooled to rt and concentrated under reduced pressure. The resulting crude was taken up in EtOAc (30 mL) and washed with water (15 mL×1). The organic phase was dried over MgSO₄, filtered, and concentrated under reduced pressure to afford the title compound as a crude product to be used for the next step. LC/MS (m/z): 478 (M+H)⁺.

### Step B: 1-(Azetidin-1-yl)-2-(6-(2-((1R,2R)-2-(piperidin-4-yl)cyclopropyl)ethoxy)pyridin-3-yl)ethanone

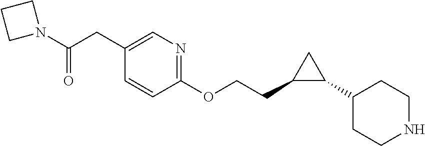 [SMILES: O=C(Cc1ccc(OCC[C@H]2C[C@@H]2C2CCNCC2)nc1)N1CCC1]

A solution of benzyl 4-((1R,2R)-2-(2-(5-(2-(azetidin-1-yl)-2-oxoethyl)pyridin-2-yloxy)ethyl)cyclopropyl)piperidine-1-carboxylate (1.14 g, 2.38 mmol) and palladium on carbon (60 mg, 0.52 mmol) in MeOH (100 mL) was stirred under 1 atm of hydrogen at rt for 1 hr. The reaction mixture was filtered through a plug of celite which was washed with MeOH. The volatiles were removed under reduced pressure to afford the title compound (640 mg, 78%) as a crude product to be used for the next step. LC/MS (m/z): 344 (M+H)⁺.

### Step C: 1-(Azetidin-1-yl)-2-(6-(2-((1R,2R)-2-(1-(5-ethylpyrimidin-2-yl)piperidin-4-yl)cyclopropyl)ethoxy)pyridin-3-yl)ethanone

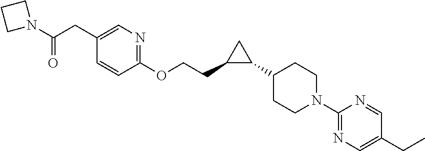 [SMILES: CCc1cnc(N2CCC([C@H]3C[C@@H]3CCOc3ccc(CC(=O)N4CCC4)cn3)CC2)nc1]

1-(Azetidin-1-yl)-2-(6-(2-((1R,2R)-2-(piperidin-4-yl)cyclopropyl)ethoxy)pyridin-3-yl)ethanone was subjected to Step E described in the synthesis of Example 1 to give the title compound. ¹H NMR (400 MHz, CDCl₃) δ 8.39 (s, 2H), 8.17 (s, 1H), 7.89 (d, 1H), 6.90 (d, 1H), 4.59 (dd, 2H), 4.32 (t, 2H), 4.23 (t, 2H), 4.06 (t, 2H), 3.43 (s, 2H), 3.06 (t, 2H), 2.57 (q, 2H), 2.33 (p, 2H), 1.92 (t, 2H), 1.60-1.32 (m, 3H), 1.24 (t, 3H), 0.96-0.84 (m, 1H), 0.73-0.62 (m, 1H), 0.48-0.31 (m, 2H). LC/MS (m/z): 450 (M+H)⁺, GPR119 Human EC₅₀: 10 nM (LCMP assay).

The example in Table 8 was synthesized according to the methods described in Example 115 employing Intermediates 5 and 23 in addition to commercially available starting materials.

TABLE 8

| Example | | Observed | GPR119 |
|---|---|---|---|
| # | Chemical Structure | Mass | Human EC₅₀ |
| 116 | 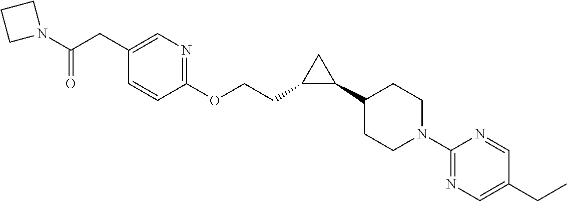 [SMILES: CCc1cnc(N2CCC([C@@H]3C[C@H]3CCOc3ccc(CC(=O)N4CCC4)cn3)CC2)nc1] | 450 [M + H]⁺ | 9.7 nM LCMP assay |

### Examples 117-119

### Preparation of 2-(2-(2-((1R,2R)-2-(1-(5-ethylpyrimidin-2-yl)piperidin-4-yl)cyclopropyl)ethoxy)-4,6-difluorophenyl)-N,N-dimethylacetamide

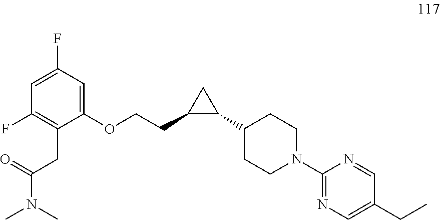 [SMILES: CCc1cnc(N2CCC([C@H]3C[C@@H]3CCOc3cc(F)cc(F)c3CC(=O)N(C)C)CC2)nc1]

### Step A: Benzyl 4-((1R,2R)-2-(2-(2-(2-(dimethylamino)-2-oxoethyl)-3,5-difluorophenoxy)ethyl)cyclopropyl)piperidine-1-carboxylate

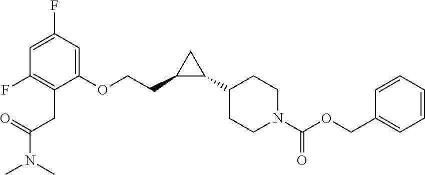 [SMILES: CN(C)C(=O)Cc1c(F)cc(F)cc1OCC[C@H]1C[C@@H]1C1CCN(C(=O)OCc2ccccc2)CC1]

Sodium t-butoxide (144 mg, 1.5 mmol) was added to a solution of benzyl 4-((1R,2R)-2-(2-hydroxyethyl)cyclopropyl)piperidine-1-carboxylate (Intermediate 4) (450 mg, 1.5 mmol) and N,N-dimethyl-2-(2,4,6-trifluorophenyl)acetamide (Intermediate 28) (400 mg, 1.9 mmol) in anhydrous THF (5 mL) that had been placed under an inert atmosphere at rt. The mixture was irradiated in the microwave at 120° C. for 1 hr. The solution was cooled to rt and concentrated under reduced pressure. The resulting crude was taken up in EtOAc (30 mL) and washed with water (15 mL×1). The organic phase was dried over MgSO₄, filtered, and concentrated under reduced pressure to afford the title compound as a crude product to be used for the next step. LC/MS (m/z): 501 (M+H)⁺.

### Step B: 2-(2,4-Difluoro-6-(2-((1R,2R)-2-(piperidin-4-yl)cyclopropyl)ethoxy)phenyl)-N,N-dimethylacetamide

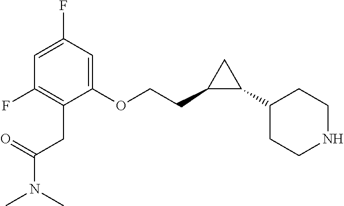 [SMILES: CN(C)C(=O)Cc1c(F)cc(F)cc1OCC[C@H]1C[C@@H]1C1CCNCC1]

A suspension Step A product (645 mg, 1.29 mmol) and palladium on carbon (56 mg, 0.52 mmol) in MeOH (60 mL) was stirred under 1 atm of hydrogen at rt for 1 hr. The reaction mixture was filtered through a plug of celite which was washed with MeOH. The volatiles were removed under reduced pressure to afford the title compound (382 mg, 81%) as a crude product to be used for the next step. LC/MS (m/z): 367 (M+H)⁺.

### Step C: 2-(2-(2-((1R,2R)-2-(1-(5-Ethylpyrimidin-2-yl)piperidin-4-yl)cyclopropyl)ethoxy)-4,6-difluorophenyl)-N,N-dimethylacetamide

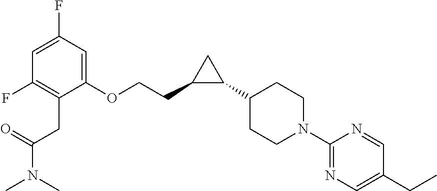 [SMILES: CCc1cnc(N2CCC([C@H]3C[C@@H]3CCOc3cc(F)cc(F)c3CC(=O)N(C)C)CC2)nc1]

2-(2,4-Difluoro-6-(2-((1R,2R)-2-(piperidin-4-yl)cyclopropyl)ethoxy)phenyl)-N,N-dimethylacetamide (Step B product) and 2-chloro-5-ethylpyrimidine were subjected to Step E described in the synthesis of Example 1 to give the title compound. ¹H NMR (400 MHz, CDCl₃) δ 8.39 (s, 2H), 6.45-6.38 (m, 2H), 4.63 (t, 2H), 3.99-3.91 (m, 2H), 3.62 (s, 2H), 3.12 (s, 3H), 3.04 (t, 2H), 2.94 (s, 3H), 2.55 (q, 2H), 1.90 (d, 2H), 1.75-1.57 (m, 2H), 1.46-1.30 (m, 2H), 1.24 (t, 3H), 0.95-0.86 (m, 1H), 0.69-0.61 (m, 1H), 0.43-0.29 (m, 3H). LC/MS (m/z): 473 (M+H)⁺, GPR119 Human EC₅₀: 12 nM (LCMP assay).

The examples in Table 9 were synthesized according to the methods described in Example 117 employing Intermediates 4, 5, and 27 in addition to commercially available starting materials.

TABLE 9

| Example | | Observed | GPR119 |
|---|---|---|---|
| # | Chemical Structure | Mass | Human EC₅₀ |
| 118 | 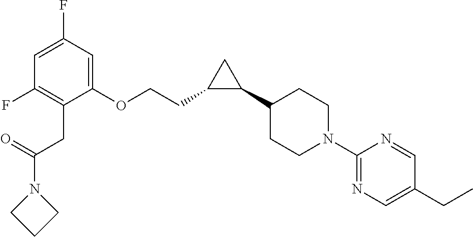 [SMILES: CCc1cnc(N2CCC([C@@H]3C[C@H]3CCOc3cc(F)cc(F)c3CC(=O)N3CCC3)CC2)nc1] | 485 [M + H]⁺ | 17 nM LCMP assay |
| 119 | 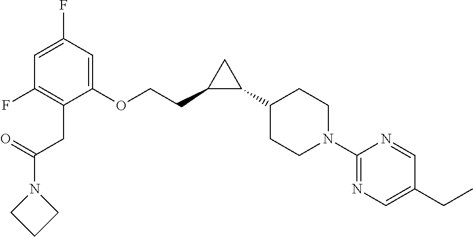 [SMILES: CCc1cnc(N2CCC([C@H]3C[C@@H]3CCOc3cc(F)cc(F)c3CC(=O)N3CCC3)CC2)nc1] | 485 [M + H]⁺ | 17 nM LCMP assay |

### Example 120

### Preparation of 1-methylcyclopropyl 4-((1S,2S)-2-(2-(5-(2-(azetidin-1-yl)-2-oxoethyl)pyridin-2-yloxy)ethyl)cyclopropyl)piperidine-1-carboxylate

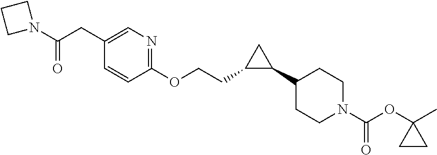 [SMILES: CC1(OC(=O)N2CCC([C@@H]3C[C@H]3CCOc3ccc(CC(=O)N4CCC4)cn3)CC2)CC1]

### Step A: 1-(Azetidin-1-yl)-2-(6-(2-((1S,2S)-2-(piperidin-4-yl)cyclopropyl)ethoxy)pyridin-3-yl)ethanone

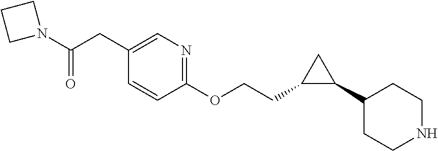 [SMILES: O=C(Cc1ccc(OCC[C@@H]2C[C@H]2C2CCNCC2)nc1)N1CCC1]

This intermediate was generated by following Steps A-B described in the synthesis of Example 115 using Intermediates 5 and 23. LC/MS (m/z): 344 (M+H)⁺.

### Step B: 4-((1S,2S)-2-(2-(5-(2-(Azetidin-1-yl)-2-oxoethyl)pyridin-2-yloxy)ethyl)cyclopropyl)piperidine-1-carboxylate

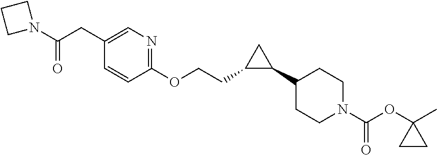 [SMILES: CC1(OC(=O)N2CCC([C@@H]3C[C@H]3CCOc3ccc(CC(=O)N4CCC4)cn3)CC2)CC1]

1-(Azetidin-1-yl)-2-(6-(2-((1S,2S)-2-(piperidin-4-yl)cyclopropyl)ethoxy)pyridin-3-yl)ethanone was subjected to Step B described in the synthesis of Example 14 to give the title compound. ¹H NMR (400 MHz, CDCl₃) δ 8.25 (s, 1H), 7.99 (d, 1H), 6.95 (d, 1H), 4.33 (t, 2H), 4.27 (t, 2H), 4.15-3.93 (m, 4H), 3.46 (s, 2H), 2.64 (t, 2H), 2.32 (p, 2H), 1.81-1.58 (m, 4H), 1.52 (s, 3H), 1.29-1.18 (m, 2H), 0.84 (t, 2H) 0.72-0.58 (m, 4H), 0.45-0.28 (m, 3H). LC/MS (m/z): 442 (M+H)⁺, GPR119 Human EC₅₀: 21 nM (LCMP assay).

### Example 121

### Preparation of 1-(azetidin-1-yl)-2-(6-(2-((1S,2S)-2-(1-(3-isopropyl-1,2,4-oxadiazol-5-yl)piperidin-4-yl)cyclopropyl)ethoxy)pyridin-3-yl)ethanone

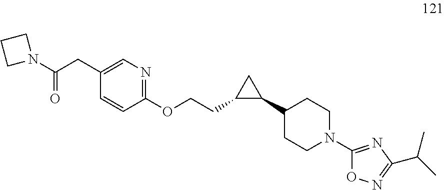 [SMILES: CC(C)c1noc(N2CCC([C@@H]3C[C@H]3CCOc3ccc(CC(=O)N4CCC4)cn3)CC2)n1]

1-(Azetidin-1-yl)-2-(6-(2-((1S,2S)-2-(piperidin-4-yl)cyclopropyl)ethoxy)pyridin-3-yl)ethanone (the product of Step A in the synthesis of Example 120) was subjected to Steps B-C described in the synthesis of Example 21 to give 1-(azetidin-1-yl)-2-(6-(2-((1S,2S)-2-(1-(3-isopropyl-1,2,4-oxadiazol-5-yl)piperidin-4-yl)cyclopropyl)ethoxy)pyridin-3-yl)ethanone. ¹H NMR (400 MHz, CDCl₃) δ 7.98 (s, 1H), 7.58 (d, 1H), 6.71 (d, 1H), 4.33 (t, 2H), 4.27 (t, 2H), 4.18-4.01 (m, 4H), 3.34 (s, 2H), 2.97 (t, 2H), 2.89 (p, 1H), 2.35 (p, 2H), 1.87-1.73 (m, 3H), 1.46-1.32 (m, 2H), 1.28 (d, 6H), 0.90-0.65 (m, 2H) 0.45-0.28 (m, 3H). LC/MS (m/z): 454 (M+H)⁺, GPR119 Human EC₅₀: 24 nM (LCMP assay).

### Example 122

### Preparation of 2-(2,4-difluoro-6-(2-((1R,2R)-2-(1-(3-isopropyl-1,2,4-oxadiazol-5-yl)piperidin-4-yl)cyclopropyl)ethoxy)phenyl)-N,N-dimethylacetamide

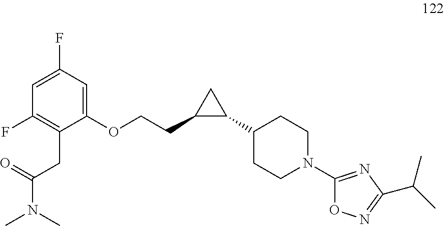 [SMILES: CC(C)c1noc(N2CCC([C@H]3C[C@@H]3CCOc3cc(F)cc(F)c3CC(=O)N(C)C)CC2)n1]

2-(2,4-Difluoro-6-(2-((1R,2R)-2-(piperidin-4-yl)cyclopropyl)ethoxy)phenyl)-N,N-dimethylacetamide (the product of Step B in the synthesis of Example 117) was subjected to Steps B-C described in the synthesis of Example 21 to give the title compound. ¹H NMR (400 MHz, CDCl₃) δ 6.45-6.38 (m, 2H), 4.09 (d, 2H), 3.99-3.91 (m, 2H), 3.62 (s, 2H), 3.12 (s, 3H), 3.00-2.91 (m, 5H), 2.87 (p, 1H), 1.80 (d, 2H), 1.78-1.58 (m, 2H), 1.46-1.32 (m, 2H), 1.26 (d, 6H), 0.85-0.60 (m, 2H) 0.43-0.29 (m, 3H). LC/MS (m/z): 477 (M+H)⁺, GPR119 Human EC₅₀: 20 nM (LCMP assay).

### Examples 123-125

### Preparation of N-(2-((1R,2S)-2-(1-(5-chloropyrimidin-2-yl)piperidin-4-yl)cyclopropyl)ethyl)-5-(1H-tetrazol-1-yl)pyrimidin-2-amine

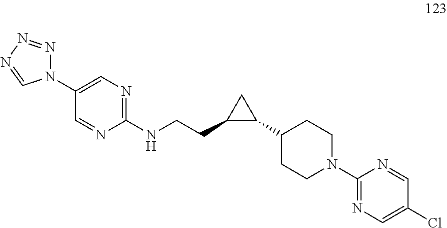 [SMILES: Clc1cnc(N2CCC([C@H]3C[C@@H]3CCNc3ncc(-n4cnnn4)cn3)CC2)nc1]

### Step A: tert-Butyl 4-((1S,2R)-2-(2-(5-nitropyrimidin-2-ylamino)ethyl)cyclopropyl)piperidine-1-carboxylate

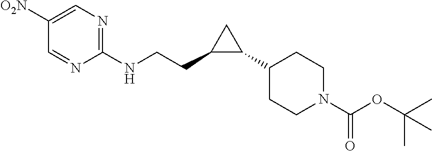 [SMILES: CC(C)(C)OC(=O)N1CCC([C@H]2C[C@@H]2CCNc2ncc([N+](=O)[O-])cn2)CC1]

tert-Butyl 4-((1S,2R)-2-(2-aminoethyl)cyclopropyl)piperidine-1-carboxylate (Intermediate 7) (600 mg, 2.2 mmol), 2-chloro-5-nitropyrimidine (427 mg, 2.6 mmol), and potassium carbonate (460 mg, 3.3 mmol) were stirred in DMF (5 mL) at rt for 12 hrs. The mixture was filtered, washing with EtOAc. The resulting organic layer was washed with saturated aqueous ammonium chloride solution (10 mL×1), dried over MgSO₄, filtered, and concentrated under reduced pressure to afford the title compound (730 mg, 85%) as a crude product to be used for the next step. LC/MS (m/z): 392 (M+H)⁺.

### Step B: tert-Butyl 4-((1S,2R)-2-(2-(5-aminopyrimidin-2-ylamino)ethyl)cyclopropyl)piperidine-1-carboxylate

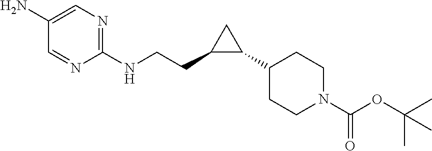 [SMILES: CC(C)(C)OC(=O)N1CCC([C@H]2C[C@@H]2CCNc2ncc(N)cn2)CC1]

A mixture of tert-butyl 4-((1S,2R)-2-(2-(5-nitropyrimidin-2-ylamino)ethyl)cyclopropyl)piperidine-1-carboxylate (730 mg, 1.86 mmol) and palladium on carbon (40 mg, 0.38 mmol) in MeOH (20 mL) was stirred under 1 atm of H₂ at rt for 2 hrs. The mixture was filtered through celite washing with MeOH and the volatiles removed under reduced pressure to afford the title compound (600 mg, 94%) as a crude product to be used for the next step. LC/MS (m/z): 362 (M+H)⁺.

### Step C: tert-Butyl 4-((1S,2R)-2-(2-(5-(1H-tetrazol-1-yl)pyrimidin-2-ylamino)ethyl)cyclopropyl)piperidine-1-carboxylate

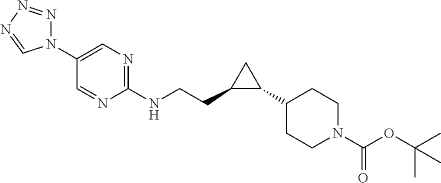 [SMILES: CC(C)(C)OC(=O)N1CCC([C@H]2C[C@@H]2CCNc2ncc(-n3cnnn3)cn2)CC1]

Triethyl orthoformate (236 mg, 1.6 mmol) was added to a stirring solution of tert-butyl 4-((1S,2R)-2-(2-(5-aminopyrimidin-2-ylamino)ethyl)cyclopropyl)piperidine-1-carboxylate (400 mg, 1.0 mmol) and sodium azide (144 mg, 2.2 mmol) in glacial acetic acid (5 mL). The reaction vessel was fitted with a reflux condenser and heated at 100° C. for 4 hrs. The reaction mixture was cooled to rt and concentrated under reduced pressure and the resulting crude partitioned between EtOAc (20 mL) and saturated aqueous sodium bicarbonate solution (20 mL). The layers were cut and the aqueous phase extracted with EtOAc (10 mL×2). The combined organic layers were dried over MgSO₄, filtered, and concentrated under reduced pressure. The residue was purified by MPLC (PE: EtOAc=1:1) to give the title compound (280 mg, 61%). LC/MS (m/z): 415 (M+H)⁺.

### Step D: N-(2-((1R,2S)-2-(piperidin-4-yl)cyclopropyl)ethyl)-5-(1H-tetrazol-1-yl)pyrimidin-2-amine hydrochloride

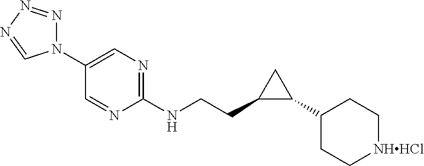

A solution of 4 M HCl in dioxane (1.7 mL, 6.8 mmol) was added to a solution of tert-butyl 4-((1S,2R)-2-(2-(5-(1H-tetrazol-1-yl)pyrimidin-2-ylamino)ethyl)cyclopropyl)piperidine-1-carboxylate (280 mg, 0.68 mmol) in DCM (5 mL). This mixture was stirred at rt for 1 hr. The reaction mixture was subsequently concentrated under reduced pressure to afford the title compound (220 mg, 92%) as a crude product to be used for the next step. LC/MS (m/z): 315 (M+H)⁺.

### Step E: N-(2-((1R,2S)-2-(1-(5-chloropyrimidin-2-yl)piperidin-4-yl)cyclopropyl)ethyl)-5-(1H-tetrazol-1-yl)pyrimidin-2-amine

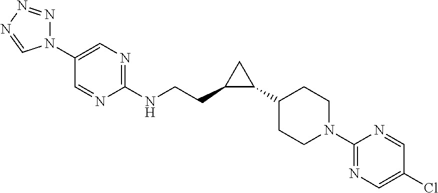 [SMILES: Clc1cnc(N2CCC([C@H]3C[C@@H]3CCNc3ncc(-n4cnnn4)cn3)CC2)nc1]

N-(2-((1R,2S)-2-(piperidin-4-yl)cyclopropyl)ethyl)-5-(1H-tetrazol-1-yl)pyrimidin-2-amine hydrochloride (Step D product) was subjected to Step E described in the synthesis of Example 1 to give the title compound. ¹H NMR (400 MHz, CDCl₃) δ 8.87 (s, 1H), 8.52 (s, 2H), 8.19 (s, 2H), 5.71 (s, 1H), 4.66 (d, 1H), 3.55 (q, 2H), 2.81 (t, 2H), 1.81 (d, 1H), 1.66-1.59 (m, 1H), 1.52-1.46 (m, 1H), 1.38-1.24 (m, 2H), 0.87-0.86 (m, 1H), 0.66-0.62 (m, 1H), 0.45-0.30 (m, 3H). LC/MS (m/z): 427 (M+H)⁺, GPR119 Human EC₅₀: 10 nM (LCMP assay).

The examples in Table 10 were synthesized according to the methods described in Example 123 employing Intermediate 7 and 9 in addition to commercially available starting materials.

TABLE 10

| Example | | Observed | GPR119 |
|---|---|---|---|
| # | Chemical Structure | Mass | Human EC₅₀ |
| 124 | 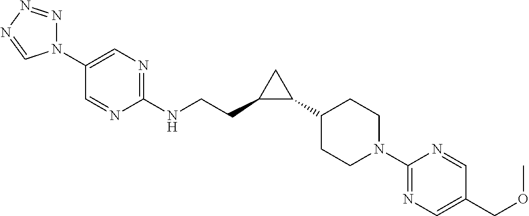 [SMILES: COCc1cnc(N2CCC([C@H]3C[C@@H]3CCNc3ncc(-n4cnnn4)cn3)CC2)nc1] | 437 [M + H]⁺ | 5.2 nM HTRF assay |
| 125 | 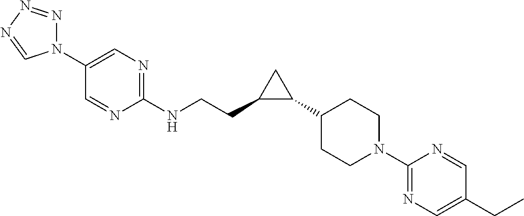 [SMILES: CCc1cnc(N2CCC([C@H]3C[C@@H]3CCNc3ncc(-n4cnnn4)cn3)CC2)nc1] | 421 [M + H]⁺ | 18 nM LCMP assay |

### Example 126

### Preparation of N-(2-((1S,2R)-2-(1-(3-isopropyl-1,2,4-oxadiazol-5-yl)piperidin-4-yl)cyclopropyl)ethyl)-5-(1H-tetrazol-1-yl)pyrimidin-2-amine

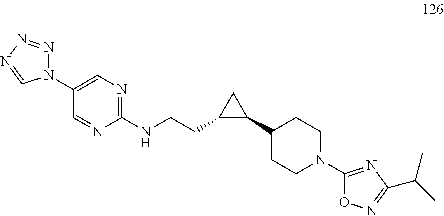 [SMILES: CC(C)c1noc(N2CCC([C@@H]3C[C@H]3CCNc3ncc(-n4cnnn4)cn3)CC2)n1]

### Step A: N-(2-((1S,2R)-2-(piperidin-4-yl)cyclopropyl)ethyl)-5-(1H-tetrazol-1-yl)pyrimidin-2-amine hydrochloride

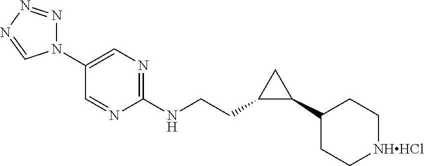

This intermediate was generated by following Steps A-D described in the synthesis of Example 123 using Intermediate 8 and commercially available starting materials. LC/MS (m/z): 315 (M+H)⁺.

### Step B: N-(2-((1S,2R)-2-(1-(3-isopropyl-1,2,4-oxadiazol-5-yl)piperidin-4-yl)cyclopropyl)ethyl)-5-(1H-tetrazol-1-yl)pyrimidin-2-amine

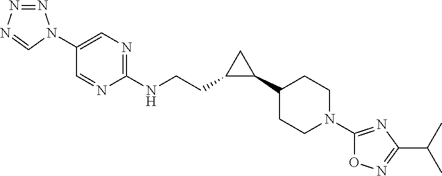 [SMILES: CC(C)c1noc(N2CCC([C@@H]3C[C@H]3CCNc3ncc(-n4cnnn4)cn3)CC2)n1]

N-(2-((1S,2R)-2-(piperidin-4-yl)cyclopropyl)ethyl)-5-(1H-tetrazol-1-yl)pyrimidin-2-amine hydrochloride was subjected to Steps B-C described in the synthesis of Example 21 to give the title compound. ¹H NMR (400 MHz, CDCl₃) δ 8.87 (s, 1H), 8.52 (s, 2H), 5.75 (s, 1H), 4.10 (d, 2H), 3.55 (q, 2H), 3.00 (t, 2H), 2.88 (m, 1H), 1.82 (d, 2H), 1.66-1.37 (m, 4H), 1.28 (s, 3H), 1.27 (s, 3H), 0.85-0.75 (m, 1H), 0.65-0.55 (m, 1H), 0.45-0.30 (m, 3H). LC/MS (m/z): 425 (M+H)⁺, GPR119 Human EC₅₀: 16 nM (LCMP assay).

### Example 127

### Preparation of (1S,2R)-2-(1-(5-chloropyrimidin-2-yl)piperidin-4-yl)-1-(2-(4-(methylsulfonyl)phenoxy)ethyl)cyclopropanecarbonitrile

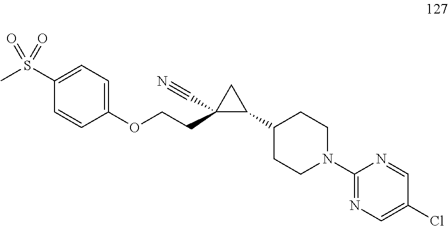 [SMILES: CS(=O)(=O)c1ccc(OCC[C@@]2(C#N)C[C@@H]2C2CCN(c3ncc(Cl)cn3)CC2)cc1]

### Step A: tert-Butyl 4-(2-cyano-3-ethoxy-3-oxoprop-1-enyl)piperidine-1-carboxylate

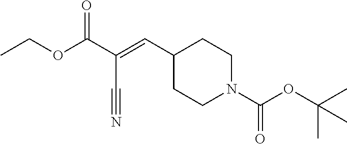 [SMILES: CCOC(=O)/C(C#N)=C/C1CCN(C(=O)OC(C)(C)C)CC1]

A mixture of tert-butyl 4-formylpiperidine-1-carboxylate (2.5 g, 11.7 mmol), ethyl cyanoacetate (1.37 mL, 12.9 mmol), glacial acetic acid (0.71 mL, 12.3 mmol), and ammonium acetate (450 mg, 5.86 mmol) in toluene (50 mL) was stirred and heated at 80° C. for 6 hrs. The reaction mixture was cooled to rt and diluted with EtOAc (30 mL) and water (50 mL). The layers were cut and the aqueous phase extracted with EtOAc (50 mL×1). The combined organic layers were washed with 1 M aqueous sodium hydroxide solution (50 mL×1), dried over MgSO₄, filtered, and concentrated under reduced pressure. The residue was loaded onto a silica column (KP-Sil 100 g SNAP column, Biotage system) eluting with a range of 15-35% EtOAc/Heptane over 12 CV to give the desired compound (2.27 g, 63%). LC/MS (m/z): 309 (M+H)⁺.

### Step B: tert-Butyl 4-(2-cyano-2-(ethoxycarbonyl)cyclopropyl)piperidine-1-carboxylate

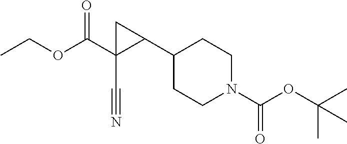 [SMILES: CCOC(=O)C1(C#N)CC1C1CCN(C(=O)OC(C)(C)C)CC1]

Sodium hydride (300 mg, 8.28 mmol) was added to a stirring solution of trimethylsulfoxonium iodide (1.82 g, 8.28 mmol) in anhydrous DMSO (15 mL) that had been placed under an inert atmosphere at rt. After ageing this mixture for 90 min a solution of tert-butyl 4-(2-cyano-3-ethoxy-3-oxoprop-1-enyl)piperidine-1-carboxylate (Step A product, 2.27 g, 8.28 mmol) in anhydrous DMSO (15 mL) was slowly introduced via syringe. The reaction was stirred at rt for 2 hrs then diluted with EtOAc (30 mL) and neutralized by the addition of saturated aqueous ammonium chloride solution (30 mL). The layers were cut and the aqueous phase extracted with EtOAc (20 mL×2). The combined organic layers were dried over MgSO₄, filtered, and concentrated under reduced pressure. The residue was loaded onto a silica column (KP-Sil 100 g SNAP column, Biotage system) eluting with a range of 15-20% EtOAc/Heptane over 12 CV to give the desired compound (1.53 g, 65%). LC/MS (m/z): 323 (M+H)⁺.

### Step C: Ethyl 2-(1-(5-chloropyrimidin-2-yl)piperidin-4-yl)-1-cyanocyclopropanecarboxylate

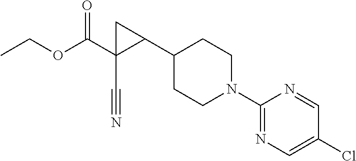 [SMILES: CCOC(=O)C1(C#N)CC1C1CCN(c2ncc(Cl)cn2)CC1]

tert-Butyl 4-(2-cyano-2-(ethoxycarbonyl)cyclopropyl)piperidine-1-carboxylate was subjected to Steps D-E described in the synthesis of Example 1 to give the title compound. LC/MS (m/z): 335 (M+H)⁺.

### Step D: 2-(1-(5-Chloropyrimidin-2-yl)piperidin-4-yl)-1-(hydroxymethyl)cyclopropanecarbonitrile

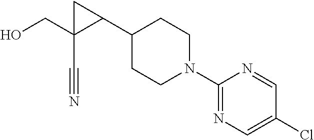 [SMILES: N#CC1(CO)CC1C1CCN(c2ncc(Cl)cn2)CC1]

Sodium borohydride (790 mg, 20.9 mmol) was added to a solution of ethyl 2-(1-(5-chloropyrimidin-2-yl)piperidin-4-yl)-1-cyanocyclopropanecarboxylate (Step C product, 700 mg, 2.09 mmol) in THF (12 mL) and water (600 uL). The reaction was aged at rt for 16 hrs then quenched through the dropwise addition of 1 M aqueous hydrogen chloride solution (22 mL). The resulting mixture was extracted with EtOAc (20 mL×3) and the combined organic layers were dried over MgSO₄, filtered, and concentrated under reduced pressure. The residue was loaded onto a silica column (KP-Sil 100 g SNAP column, Biotage system) eluting with a range of 20-60% EtOAc/Heptane over 12 CV to give the desired compound (439 mg, 72%). LC/MS (m/z): 293 (M+H)⁺.

### Step E: 2-(1-(5-Chloropyrimidin-2-yl)piperidin-4-yl)-1-formylcyclopropanecarbonitrile

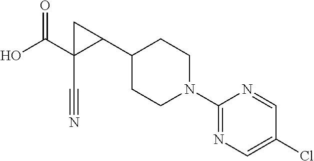 [SMILES: N#CC1(C(=O)O)CC1C1CCN(c2ncc(Cl)cn2)CC1]

A solution of 2-(1-(5-chloropyrimidin-2-yl)piperidin-4-yl)-1-(hydroxymethyl)cyclopropanecarbonitrile (Step D product, 146 mg, 0.5 mmol) in DCM (1 mL) was added to a solution of oxalyl chloride (50 uL, 0.55 mmol) and DMSO (80 uL, 1.1 mmol) in DCM (1 mL) that had been cooled to −50° C. and placed under an inert atmosphere. This mixture was aged at −50° C. for 15 min after which triethylamine (350 uL, 2.5 mmol) was introduced. The reaction was warmed to rt then diluted with DCM (5 mL) and water (5 mL). The layers were cut and the aqueous phase extracted with DCM (5 mL×2). The combined organic layers were dried over MgSO₄, filtered, and concentrated under reduced pressure. The residue was loaded onto a silica column (KP-Sil 10 g SNAP column, Biotage system) eluting with a range of 20-60% EtOAc/Hex over 12 CV to give the title compound (100 mg, 69%). LC/MS (m/z): 291 (M+H)⁺.

### Step F: 2-(1-(5-Chloropyrimidin-2-yl)piperidin-4-yl)-1-vinylcyclopropanecarbonitrile

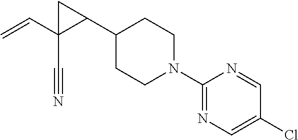 [SMILES: C=CC1(C#N)CC1C1CCN(c2ncc(Cl)cn2)CC1]

A 1 M solution of LiHMDS in THF (850 uL, 0.85 mmol) was introduced to a solution of methyltriphenylphosphonium bromide (316 mg, 0.89 mmol) in anhydrous toluene (1 mL) that had been cooled to 0° C. and placed under an inert atmosphere. After ageing for 20 min this mixture was transferred via cannula to a second flask containing a stirring mixture of 2-(1-(5-chloropyrimidin-2-yl)piperidin-4-yl)-1-formylcyclopropanecarbonitrile (Step E product, 99 mg, 0.34 mmol) in anhydrous toluene (1 mL) at rt. The reaction mixture was stirred at rt for 2 hrs then was diluted with DCM (10 mL) and saturated aqueous ammonium chloride solution (10 mL). The layers were cut and the aqueous phase extracted with DCM (10 mL×2). The combined organic layers were dried over MgSO₄, filtered, and concentrated under reduced pressure. The residue was loaded onto a silica column (KP-Sil 10 g SNAP column, Biotage system) eluting with a range of 10-40% EtOAc/Hex over 12 CV to give the title compound (100 mg, 69%). LC/MS (m/z): 289 (M+H)⁺.

### Step G: 2-(1-(5-Chloropyrimidin-2-yl)piperidin-4-yl)-1-(2-hydroxyethyl)cyclopropanecarbonitrile

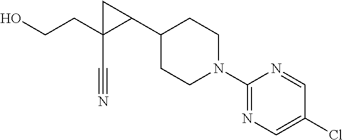 [SMILES: N#CC1(CCO)CC1C1CCN(c2ncc(Cl)cn2)CC1]

A 2 M solution of borane.dimethyl sulfide in THF (150 uL, 0.31 mmol) was introduced to a solution of 2-(1-(5-chloropyrimidin-2-yl)piperidin-4-yl)-1-vinylcyclopropanecarbonitrile (Step F product, 18 mg, 0.062 mmol) in THF (500 uL) that had been cooled to 0° C. and placed under an inert atmosphere. The reaction mixture was stirred at 0° C. for 2 hrs after which a 5 M solution of sodium hydroxide (100 uL, 0.5 mmol) and hydrogen peroxide (100 uL, 1.1 mmol) were added. The reaction mixture was aged at rt for 1 hr then was diluted with EtOAc (5 mL) and water (5 mL). The layers were cut and the aqueous phase extracted with EtOAc (5 mL×2). The combined organic layers were dried over MgSO₄, filtered, and concentrated under reduced pressure. The residue was loaded onto a silica column (KP-Sil 10 g SNAP column, Biotage system) eluting with a range of 25-65% EtOAc/Hex over 12 CV to give the title compound (10 mg, 49%). LC/MS (m/z): 307 (M+H)⁺.

### Step H: (1S,2R)-2-(1-(5-Chloropyrimidin-2-yl)piperidin-4-yl)-1-(2-(4-(methylsulfonyl)phenoxy)ethyl)cyclopropanecarbonitrile

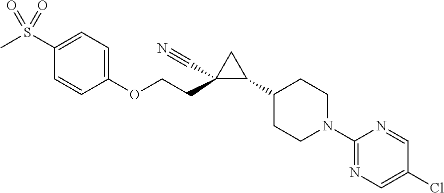 [SMILES: CS(=O)(=O)c1ccc(OCC[C@@]2(C#N)C[C@@H]2C2CCN(c3ncc(Cl)cn3)CC2)cc1]

2-(1-(5-Chloropyrimidin-2-yl)piperidin-4-yl)-1-(2-hydroxyethyl)cyclopropanecarbonitrile (Step G product) was subjected to Step A described in the synthesis of Example 24 to give the title compound. ¹H NMR (500 MHz, CDCl₃) δ 8.21 (s, 2H), 7.88 (d, 2H), 7.03 (d, 2H), 4.69 (dd, 2H), 4.32-4.19 (m, 2H), 3.06 (s, 3H), 2.88 (t, 2H), 2.22-2.16 (m, 1H), 2.01 (d, 1H), 1.87-1.78 (m, 2H), 1.46-1.26 (m, 4H), 1.03 (t, 1H), 0.88 (t, 1H), 0.65-0.55 (m, 1H), 0.45-0.30 (m, 3H). LC/MS (m/z): 461 (M+H)⁺, GPR119 Human EC₅₀: 3.0 nM (HTRF assay).

### Example 128

### Preparation of N-(2-((1R,2S)-2-(1-(5-chloropyrimidin-2-yl)piperidin-4-yl)cyclopropyl)ethyl)-4-(methylsulfonyl)aniline

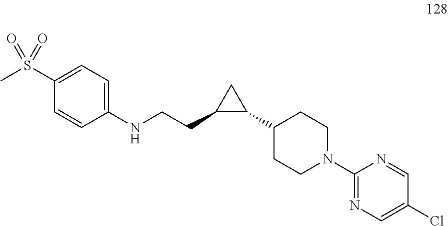 [SMILES: CS(=O)(=O)c1ccc(NCC[C@H]2C[C@@H]2C2CCN(c3ncc(Cl)cn3)CC2)cc1]

### Step A: tert-Butyl 4-((1S,2R)-2-(2-(4-(methylsulfonyl)phenylamino)ethyl)cyclopropyl)piperidine-1-carboxylate

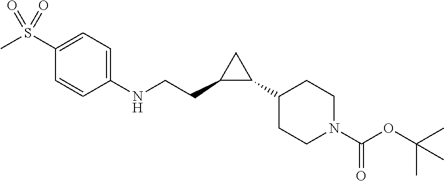 [SMILES: CC(C)(C)OC(=O)N1CCC([C@H]2C[C@@H]2CCNc2ccc(S(C)(=O)=O)cc2)CC1]

DBU (40 uL, 0.27 mmol) was added to a solution of tert-butyl 4-((1S,2R)-2-(2-aminoethyl)cyclopropyl)piperidine-1-carboxylate (Intermediate 7) (48 mg, 0.18 mmol) and 1-fluoro-4-(methylsulfonyl)benzene (37 mg, 0.21 mmol) in NMP (1 mL) that had been placed under an inert atmosphere at rt. The mixture was irradiated in the microwave at 120° C. for 1 hr. The solution was cooled to rt and concentrated under reduced pressure. The resulting crude was taken up in EtOAc (30 mL) and washed with water (15 mL×1). The organic phase was dried over MgSO₄, filtered, and concentrated under reduced pressure. The residue was loaded onto a silica column (KP-Sil 10 g SNAP column, Biotage system) eluting with a range of 0-65% EtOAc/Hex over 12 CV to give the title compound (41 mg, 53%). LC/MS (m/z): 423 (M+H)⁺.

### Step B: N-(2-((1R,2S)-2-(1-(5-chloropyrimidin-2-yl)piperidin-4-yl)cyclopropyl)ethyl)-4-(methylsulfonyl)aniline

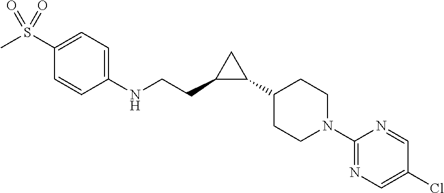 [SMILES: CS(=O)(=O)c1ccc(NCC[C@H]2C[C@@H]2C2CCN(c3ncc(Cl)cn3)CC2)cc1]

tert-Butyl 4-((1S,2R)-2-(2-(4-(methylsulfonyl)phenylamino)ethyl)cyclopropyl)piperidine-1-carboxylate (Step A product) was subjected to Steps D-E described in the synthesis of Example 1 to give the title compound. ¹H NMR (500 MHz, CDCl₃) δ 8.22 (s, 2H), 7.73 (d, 2H), 6.62 (d, 2H), 4.67 (d, 2H), 3.25 (t, 2H), 3.02 (s, 3H), 2.84 (t, 2H), 1.82 (d, 2H), 1.87-1.78 (m, 2H), 1.68-1.49 (m, 2H), 1.34 (p, 2H), 0.92-0.83 (m, 1H), 0.65-0.58 (m, 1H), 0.44-0.35 (m, 3H). LC/MS (m/z): 435 (M+H)⁺, GPR119 Human EC₅₀: 6.7 nM (HTRF assay).

Compounds of the present invention were shown to be biologically active in one or more of the following assays:

Measurement of GPR119 Signaling Using LANCE 384-Well cAMP Kit (LCMP Assay)

Human embryonic kidney (HEK) 293 cell lines stably transfected with human GPR119 were maintained in DMEM media containing FBS, penicillin-streptomycin, HEPES, and hygromycin. For the cAMP assay, the transfected cells were harvested using a non-enzymatic cell dissociation solution (GIBCO 2672), pelleted and resuspended in stimulation buffer (DMEM, 25 mM Hepes, 0.1% BSA, pH 7.4 in the presence of 100 μM phosphodiesterase inhibitors). The adenylate cyclase assay was constructed following the LANCE™ cAMP Kit (Perkin Elmer, AD0264) instructions. Briefly, cells with Alexa Fluor® 647-anti cAMP antibody were incubated with 10 point series diluted test article in stimulation buffer with a final concentration of 2.5% DMSO for 45 minutes. The reaction was stopped by incubating with the supplied detection buffer containing the europium chelate of the Eu-SA/Biotin-cAMP tracer for 3 hours. The assay was performed in duplicate in a 384 well plate for duplicate plates. Fluorescence at 665 nm was measured using a PHERAstar instrument. Basal activity was determined using a DMSO control and maximum response was defined as cAMP stimulation produced by an internal agonist control. Standard cAMP concentrations were assayed concurrently for conversion of fluorescence signal to cAMP level. The data was analyzed using 4-parameter curve fit in Microsoft Excel.

Measurement of GPR119 Signaling Using a Cyclic AMP (cAMP) Homogenous Time Resolved Fluorescence (HTRF) Assay

Chinese hamster ovary (CHO) cell lines stably transfected with the permissive guanine nucleotide binding protein alpha 15 (Gals) and murine GPR119 were maintained in DMEM media containing FBS, penicillin-streptomycin, puromycin, and G418 (geneticin). Alternatively, human embryonic kidney (HEK)293 Flp-In cells (Invitrogen, Carlsbad, Calif.) were stably transfected with a human SNP variant (S309L) of GPR119 and maintained in DMEM media containing FBS, penicillin-streptomycin, and hygromycin. Agonist activation of the GPR119 receptor was measured in receptor transfected cells described above, treated with compounds of this invention, using a commercial homogenous time resolved fluorescence (HTRF) kit for measurement of cAMP (CisBio, Bedford, Mass.). The assay was performed in 96-well half-volume plates (murine) or 384-well plates (human) following the manufacturers instructions. Briefly, suspended cells were incubated with a dose titration of test compound at RT for 60 min, lysed, and incubated with HTRF reagents for an additional 60 min. The plate was read using an Envision multilabel reader (Perkin Elmer) adjusted to read time resolved fluorescence and the cAMP concentrations were extrapolated from a cAMP calibration curve. GPR119 agonists will exhibit a concentration-dependent increase in intracellular cAMP. The concentration of test compound required to stimulate a half-maximal response (EC50), and efficacy as compared to an internal agonist control, was determined from a sigmoidal 4-parameter curve fit of the resulting plot of normalized activity versus compound concentration.

### Evaluation of Glucose Dependent Insulin Secretion (GDIS) in Static Isolated Mouse Islets.

Pancreatic islets of Langerhans were isolated from the pancreata of 10-12 wk-old C57BL/6 mice by collagenase digestion and discontinuous Ficoll gradient separation, a modification of the original method of Lacy and Kostianovsky (Lacy & Kostianovsky, 1967 Diabetes 16-35-39). The islets were cultured overnight in RPMI 1640 medium (11 mM glucose, 10% FCS) before experimental treatment. The acute effects of compounds of this invention on GDIS were determined by 60-min static incubation with islets in Krebs-Ringers' bicarbonate (KRB) medium. The KRB medium contained, in mM, 143.5 Na⁺, 5.8 K⁺, 2.5 Ca²⁺, 1.2 Mg²⁺, 124.1 Cl⁻, 1.2 PO₄³⁻, 1.2 SO₄²⁺, 25 CO₃²⁻, and 10 HEPES, pH 7.4, in addition to 2 mg/ml bovine serum albumin, and either 2 (G2) or 16 (G16) mM glucose (pH 7.4). The static incubation was performed with round-bottomed 96-well plates (one islet/well with 200 μl KRB medium). The compounds were added to KRB medium just before the initiation of the 60-min incubation. Insulin concentration in aliquots of the incubation buffer was measured by the ultra-sensitive rat insulin EIA kit from ALPCO Diagnostics (Windham, N.H.).

### Example of a Pharmaceutical Formulation

As a specific embodiment of an oral composition of a compound of the present invention, 50 mg of any of the examples is formulated with sufficient finely divided lactose to provide a total amount of 580 to 590 mg to fill a size 0 hard gelatin capsule.

While the invention has been described and illustrated in reference to specific embodiments thereof, various changes, modifications, and substitutions can be made therein without departing from the invention. For example, alternative effective dosages may be applicable, based upon the responsiveness of the patient being treated. Likewise, the pharmacologic response may vary depending upon the particular active compound selected, formulation and mode of administration. All such variations are included within the present invention.

1. A compound represented by the formula: 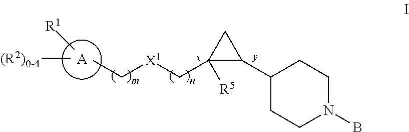
or a pharmaceutically acceptable salt thereof, wherein:
bonds x and y are in trans orientation in reference to one another;
ring A represents a 5- or 6-membered aryl or heteroaryl ring, the heteroaryl ring containing 1-4 heteroatoms, 1-4 of which are nitrogen atoms, and 0-1 of which are oxygen or sulfur atoms;
X¹ is oxygen or nitrogen;
m is an integer selected from 0-3;
n is an integer selected from 1-3; where m+n can be no more than 5;
R¹ is selected from:
halogen;
C₁₋₆alkyl;
OC₁₋₆alkyl;
C(O)C₁₋₆alkyl;
C(O)C₃₋₆cycloalkyl;
C(O)NHC₁₋₆alkyl;
S(O)₀₋₂C₁₋₆alkyl;
SO₂C₃₋₆cycloalkyl;
SO₂NR^{b}R^{c}, wherein R^{b} and R^{c} are independently selected from H or C₁₋₆alkyl; —C₁₋₂alkylCONR^{d}R^{e} wherein R^{d} and R^{e} are independently selected from the group consisting of H, C₁₋₆alkyl, C₃₋₆cycloalkyl, haloC₁₋₆alkyl, haloC₃₋₆cycloalkyl, C(O)NH₂, C₁₋₆alkoxy, and C₃₋₆cycloalkylC₁₋₆alkoxy; wherein R^{d} and R^{e}, if individually alkyl, alkoxy or C(O)NH₂, can together form a 4-6-membered saturated heterocyclic ring having 1 nitrogen atom which 4-6-membered ring may be optionally substituted with 1-3 substituents independently selected from halogen, hydroxy, oxo; C₁₋₆alkyl, C₁₋₆alkoxy; or CO₂C₁₋₆alkyl;
—CH₂-heteroaryl, wherein the heteroaryl is a 5-6 membered heteroaryl ring containing 1-4 heteroatoms independently selected from nitrogen or oxygen;
or a 5-6 membered heteroaryl or saturated heterocyclic ring containing 1-4 heteroatoms, 1-4 of which are nitrogen atoms, and 0-1 of which are oxygen or sulfur atoms,
wherein the R¹ alkyl, cycloalkyl, heteroaryl or heterocyclic moiety is optionally substituted with 1-3 of halogen; hydroxy; oxo; C₁₋₆alkyl; NH²; and O—C₁₋₆alkyl;
each R² is independently selected from halogen, CN, C₁₋₆alkyl or haloC₁₋₆alkyl;
R⁵ is hydrogen, C₁₋₃ alkyl, C₁₋₃alkoxy, or cyano;
B represents (a) a 6 membered aryl ring or a 5-6 membered heteroaryl ring containing 1-4 heteroatoms, 1-4 of which are nitrogen atoms, and 0-1 of which are oxygen or sulfur atoms, said ring being optionally substituted with 1-3 groups selected from R³, wherein
each R³ is independently selected from
halogen;
hydroxyl;
C₁₋₆alkyl;
C(O)OC₁₋₆alkyl;
C═O;
CN;
C₁₋₆alkoxy;
C₃₋₆cycloalkyl;
C(═O)—(O)ₙ—R′, wherein n is an integer from 0-3 and R′ is C₁₋₆alkyl or H;
5-6 membered heteroaryl ring containing 1-4 heteroatoms independently selected from nitrogen or oxygen;
C(═O)—R^{f}, wherein R^{f} is 5-6 membered heteroaryl ring containing 1-4 heteroatoms independently selected from nitrogen or oxygen;
or haloC₁₋₆alkoxy;
wherein the R³ alkyl moiety is optionally substituted with 1-3 substituents independently selected from:
halogen,
hydroxy,
C₁₋₆alkyl, or
C₁₋₆alkoxy;
or b) CO₂R⁴, wherein
R⁴ is selected from:
C₁₋₆alkyl; or
C₃₋₆ cycloalkyl,
wherein the alkyl and cycloalkyl are optionally substituted with 1-3 substituents independently selected from:
halogen,
C₁₋₆alkyl, or
C₃₋₆cycloalkyl.

2. The compound of claim 1 of formula I-a: 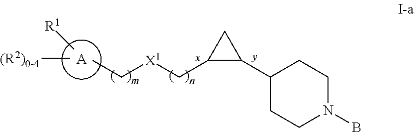
or a pharmaceutically acceptable salt thereof, wherein:
bonds x and y are in trans orientation in reference to one another;
ring A is substituted phenyl, pyridyl or pyrimidine;
X¹ is oxygen or nitrogen;
m is an integer selected from 0-3;
n is an integer selected from 1-3; where m+n can be no more than 5;
B represents a) a pyrimidine ring optionally substituted with 1-2 groups selected from R³, wherein each R³ is independently selected from:
halogen;
hydroxyl;
C₁₋₆alkyl;
C(O)OC₁₋₆alkyl;
C═O;
CN;
C₁₋₆alkoxy;
C₃₋₆cycloalkyl;
C(═O)—(O)ₙ—R′, wherein n is an integer from 0-3 and R′ is C₁₋₆alkyl or H;
5-6 membered heteroaryl ring containing 1-4 heteroatoms independently selected from nitrogen or oxygen;
C(═O)—R^{f}, wherein R^{f} is a 5-6 membered heteroaryl ring containing 1-4 heteroatoms independently selected from nitrogen or oxygen;
or haloC₁₋₆alkoxy;
wherein the R³ alkyl moiety is optionally substituted with 1-3 substituents independently selected from:
halogen,
hydroxy,
C₁₋₆alkyl, or
C₁₋₆alkoxy;
b) 1,2,4-oxadiazol optionally substituted with 1-3 substituents independently selected from:
C₁₋₆alkyl;
C₁₋₆ alkoxy; or
C₃₋₆cycloalkyl,
wherein the alkyl, alkoxy and cycloalkyl are optionally substituted with 1-3 substituents independently selected from:
halogen,
C₁₋₆alkyl, or
C₃₋₆cycloalkyl;
or c) CO₂R⁴, wherein
R⁴ is selected from:
C₁₋₆alkyl; or
C₃₋₆ cycloalkyl,
wherein the alkyl and cycloalkyl are optionally substituted with 1-3 substituents independently selected from:
halogen,
C₁₋₆alkyl, or
C₃₋₆cycloalkyl;
R¹ is selected from:
C₁₋₆alkyl;
OC₁₋₆alkyl;
C(O)C₁₋₆alkyl;
C(O)C₃₋₆cycloalkyl;
C(O)NHC₁₋₆alkyl;
S(O)₀₋₂C₁₋₆alkyl;
SO₂C₃₋₆cycloalkyl;
—C₁₋₂alkylCONR^{d}R^{e} wherein R^{d} and R^{e} are independently selected from H, C₁₋₆alkyl, C₃₋₆cycloalkyl, haloC₁₋₆alkyl, haloC₃₋₆cycloalkyl, C(O)NH₂, C₁₋₆alkoxy, or C₃₋₆cycloalkylC₁₋₆alkoxy; wherein R^{d} and R^{e}, if individually alkyl, alkoxy or C(O)NH₂, can together form a 4-6-membered saturated heterocyclic ring having 1 nitrogen atom which 4-6-membered ring may be optionally substituted with 1-3 substituents independently selected from halogen, hydroxy, oxo, C₁₋₆alkyl, C₁₋₆alkoxy; or CO₂C₁-6alkyl;
—CH₂-heteroaryl, wherein the heteroaryl is a 5-6 membered heteroaryl ring containing 1-4 heteroatoms independently selected from nitrogen or oxygen;
or a 5-6 membered heteroaryl or saturated heterocyclic ring containing 1-4 heteroatoms, 1-4 of which are nitrogen atoms, and 0-1 of which are oxygen or sulfur atoms,
wherein the R¹ alkyl, cycloalkyl, heteroaryl or heterocyclic moiety is optionally substituted with 1-3 substituents selected from: halogen; hydroxy; C₁₋₆alkyl; NH²; or O—C₁₋₆alkyl; and R² is halogen which is further selected from fluoro or chloro.

3. The compound of claim 1, or a pharmaceutically acceptable salt thereof, wherein ring A is pyridyl.

4. The compound of claim 1, or a pharmaceutically acceptable salt thereof, wherein ring A is phenyl.

5. The compound of claim 1, or a pharmaceutically acceptable salt thereof, wherein B represents (b) CO₂R⁴, wherein R⁴ is
C₁₋₆alkyl, or
C₃₋₆cycloalkyl,
wherein the alkyl and cycloalkyl are optionally substituted with 1-3 substituents independently selected from:
halogen,
C₁₋₆alkyl,
or C₃₋₆cycloalkyl.

6. The compound of claim 1, or a pharmaceutically acceptable salt thereof, wherein B is pyrimidine, optionally substituted with 1-3 substituents independently selected from:
halogen;
hydroxyl;
C₁₋₆alkyl;
C(O)OC₁₋₆alkyl;
C═O;
CN;
C₁₋₆alkoxy;
C₃₋₆cycloalkyl;
C(═O)—(O)ₙ—R′, wherein n is an integer from 0-3 and R′ is C₁₋₆alkyl or H;
5-6 membered heteroaryl ring containing 1-4 heteroatoms independently selected from nitrogen or oxygen;
C(═O)—R^{f}, wherein R^{f} is a 5-6 membered heteroaryl ring containing 1-4 heteroatoms independently selected from nitrogen or oxygen;
or haloC₁₋₆alkoxy;
wherein the alkyl moiety is optionally substituted with 1-3 substituents independently selected from:
halogen,
hydroxy,
C₁₋₆ alkyl, or
C₁₋₆ alkoxy.

7. The compound of claim 1, or a pharmaceutically acceptable salt thereof, wherein B is 1,2,4-oxadiazol optionally substituted with 1-3 substituents independently selected from
C₁₋₆alkyl;
C₁₋₆alkoxy; or
C₃₋₆cycloalkyl,
wherein the alkyl, alkoxy and cycloalkyl are optionally substituted with 1-3 substituents independently selected from:
halogen,
C₁₋₆alkyl, or
C₃₋₆cycloalkyl.

8. The compound of claim 1, or a pharmaceutically acceptable salt thereof, wherein each R³ is independently selected from halogen which is further selected from F, Cl, or Br, C₁₋₄alkyl, C₁₋₃alkoxy or C₃₋₆cycloalkyl.

9. The compound of claim 1, or a pharmaceutically acceptable salt thereof, wherein B is methoxymethylpyrimidine.

10. The compound of claim 1, or a pharmaceutically acceptable salt thereof, wherein R² is halogen which is further selected from fluoro or chloro.

11. The compound of claim 1, or a pharmaceutically acceptable salt thereof, wherein R¹ is at the 4 position and is selected from C₁₋₆alkyl; OC₁₋₆alkyl; C(O)C₁₋₆alkyl; C(O)C₃₋₆cycloalkyl; C(O)NHC₁₋₆alkyl; S(O)₀₋₂C₁₋₆alkyl; SO₂C₃₋₆cycloalkyl; SO₂NR^{b}R^{c}, wherein R^{b} and R^{c} are selected from H or C₁₋₆alkyl; or a 5-6 membered heteroaryl ring containing 1-4 heteroatoms, 1-4 of which are nitrogen atoms, and 0-1 of which are oxygen or sulfur atoms, wherein the R¹ alkyl, cycloalkyl and heteroaryl moiety is optionally substituted with 1-3 substituents independently selected from: halogen; hydroxy; C₁₋₆alkyl or O—C₁₋₆alkyl.

12. The compound of claim 1, or a pharmaceutically acceptable salt thereof, wherein R¹ is at the 4 position and is selected from CH₂CONR^{d}R^{e} wherein R^{d} and R^{e} are independently selected from H, C₁₋₆alkyl, C₃₋₆cycloalkyl, haloC₁₋₆alkyl, haloC₃₋₆cycloalkyl, C(O)NH₂, C₁₋₆alkoxy, or C₃₋₆cycloalkylC₁₋₆alkoxy; wherein R^{d} and R^{e}, if individually alkyl, alkoxy or C(O)NH₂, can together form a 4-6-membered saturated heterocyclic ring having 1 nitrogen atom which 4-6-membered ring may be optionally substituted with 1-3 substituents independently selected from halogen, hydroxy, oxo. C₁₋₆alkyl, C₁₋₆alkoxy; or CO₂C₁₋₆alkyl;

13. The compound of claim 1, or a pharmaceutically acceptable salt thereof, wherein R¹ is methylsulfonyl.

14. The compound of claim 1 which is:
5-chloro-2-(4-((1R,2S)-2-(((5-(methylsulfonyl)pyridin-2-yl)methoxy)methyl)cyclopropyl)piperidin-1-yl)pyrimidine;
5-chloro-2-(4-((1R,2S)-2-(((4-(methylsulfonyl)benzyl)oxy)methyl)cyclopropyl)piperidin-1-yl)pyrimidine;
5-chloro-2-(4-((1R,2S)-2-(((4-(ethylsulfonyl)benzyl)oxy)methyl)cyclopropyl)piperidin-1-yl)pyrimidine;
5-chloro-2-(4-((1R,2S)-2-(((2-fluoro-4-(methylsulfonyl)benzyl)oxy)methyl)cyclopropyl)piperidin-1-yl)pyrimidine;
5-chloro-2-(4-((1R,2S)-2-(((3-fluoro-4-(methylsulfonyl)benzyl)oxy)methyl)cyclopropyl)piperidin-1-yl)pyrimidine,
5-methyl-2-(4-((1R,2S)-2-(((4-(methylsulfonyl)benzyl)oxy)methyl)cyclopropyl)piperidin-1-yl)pyrimidine;
2-(4-((1R,2S)-2-(((4-(ethylsulfonyl)benzyl)oxy)methyl)cyclopropyl)piperidin-1-yl)-5-methylpyrimidine;
5-ethyl-2-(4-((1R,2S)-2-(((2-fluoro-4-(methylsulfonyl)benzyl)oxy)methyl)cyclopropyl)piperidin-1-yl)pyrimidine;
5-ethyl-2-(4-((1R,2S)-2-(((4-(ethylsulfonyl)benzyl)oxy)methyl)cyclopropyl)piperidin-1-yl)pyrimidine;
5-ethyl-2-(4-((1R,2S)-2-(((4-(methylsulfonyl)benzyl)oxy)methyl)cyclopropyl)piperidin-1-yl)pyrimidine;
2-(4-((1R,2S)-2-(((2-fluoro-4-(methylsulfonyl)benzyl)oxy)methyl)cyclopropyl)piperidin-1-yl)-5-(methoxymethyl)pyrimidine;
5-(methoxymethyl)-2-(4-((1R,2S)-2-(((4-(methylsulfonyl)benzyl)oxy)methyl)cyclopropyl)piperidin-1-yl)pyrimidine;
2-(4-((1R,2S)-2-(((4-(ethylsulfonyl)benzyl)oxy)methyl)cyclopropyl)piperidin-1-yl)-5-(methoxymethyl)pyrimidine;
1-methylcyclopropyl 4-((1R,2S)-2-((2-fluoro-4-(methylsulfonyl)benzyloxy)methyl)cyclopropyl)piperidine-1-carboxylate;
cyclopropyl 4-((1R,2S)-2-(((3-fluoro-4-(methylsulfonyl)benzyl)oxy)methyl)cyclopropyl)piperidine-1-carboxylate;
cyclopropyl 4-((1R,2S)-2-(((4-(methylsulfonyl)benzyl)oxy)methyl)cyclopropyl)piperidine-1-carboxylate;
1-methylcyclopropyl 4-((1R,2S)-2-(((3-fluoro-4-(methylsulfonyl)benzyl)oxy)methyl)cyclopropyl)piperidine-1-carboxylate,
1-methylcyclopropyl 4-((1R,2S)-2-(((4-(methylsulfonyl)benzyl)oxy)methyl)cyclopropyl)piperidine-1-carboxylate;
isopropyl 4-((1R,2S)-2-(((4-(methylsulfonyl)benzyl)oxy)methyl)cyclopropyl)piperidine-1-carboxylate;
isopropyl 4-((1R,2S)-2-(((3-fluoro-4-(methylsulfonyl)benzyl)oxy)methyl)cyclopropyl)piperidine-1-carboxylate;
3-isopropyl-5-(4-((1R,2S)-2-((4-(methylsulfonyl)benzyloxy)methyl)cyclopropyl)piperidin-1-yl)-1,2,4-oxadiazole;
5-(4-((1R,2S)-2-(((3-fluoro-4-(methylsulfonyl)benzyl)oxy)methyl)cyclopropyl)piperidin-1-yl)-3-isopropyl-1,2,4-oxadiazole;
1-methylcyclopropyl 4-((1R,2S)-2-((4-(2-(azetidin-1-yl)-2-oxoethyl)-2-fluorobenzyloxy)methyl)cyclopropyl)piperidine-1-carboxylate;
5-chloro-2-(4-((1S,2S)-2-(2-(3-fluoro-4-(methylsulfonyl)phenoxy)ethyl)cyclopropyl)piperidin-1-yl)pyrimidine;
5-chloro-2-(4-((1R,2R)-2-(2-(3-fluoro-4-(methylsulfonyl)phenoxy)ethyl)cyclopropyl)piperidin-1-yl)pyrimidine;
5-ethyl-2-(4-((1S,2S)-2-(2-(2-fluoro-4-(methylsulfonyl)phenoxy)ethyl)cyclopropyl)piperidin-1-yl)pyrimidine;
5-ethyl-2-(4-((1R,2R)-2-(2-(3-fluoro-4-(methylsulfonyl)phenoxy)ethyl)cyclopropyl)piperidin-1-yl)pyrimidine;
5-ethyl-2-(4-((1R,2R)-2-(2-(2-fluoro-4-(methylsulfonyl)phenoxy)ethyl)cyclopropyl)piperidin-1-yl)pyrimidine;
5-chloro-2-(4-((1S,2S)-2-(2-(2-fluoro-4-(methylsulfonyl)phenoxy)ethyl)cyclopropyl)piperidin-1-yl)pyrimidine;
5-chloro-2-(4-((1R,2R)-2-(2-(2-fluoro-4-(methylsulfonyl)phenoxy)ethyl)cyclopropyl)piperidin-1-yl)pyrimidine;
5-ethyl-2-(4-((1S,2S)-2-(2-(3-fluoro-4-(methylsulfonyl)phenoxy)ethyl)cyclopropyl)piperidin-1-yl)pyrimidine;
5-ethyl-2-(4-((1S,2S)-2-(2-(4-(ethylsulfonyl)-2-fluorophenoxy)ethyl)cyclopropyl)piperidin-1-yl)pyrimidine;
2-(4-((1R,2R)-2-(2-(3-fluoro-4-(methylsulfonyl)phenoxy)ethyl)cyclopropyl)piperidin-1-yl)-5-(methoxymethyl)pyrimidine;
1-(azetidin-1-yl)-2-(4-(2-((1R,2R)-2-(1-(5-chloropyrimidin-2-yl)piperidin-4-yl)cyclopropyl)ethoxy)-2-fluorophenyl)ethanone;
1-(azetidin-1-yl)-2-(4-(2-((1R,2R)-2-(1-(5-chloropyrimidin-2-yl)piperidin-4-yl)cyclopropyl)ethoxy)-3-fluorophenyl)ethanone;
1-(azetidin-1-yl)-2-(4-(2-((1S,2S)-2-(1-(5-ethylpyrimidin-2-yl)piperidin-4-yl)cyclopropyl)ethoxy)-2-fluorophenyl)ethanone;
5-chloro-2-(4-((1R,2R)-2-(2-(3-fluoro-4-(1H-tetrazol-1-yl)phenoxy)ethyl)cyclopropyl)piperidin-1-yl)pyrimidine;
5-ethyl-2-(4-((1R,2R)-2-(2-(3-fluoro-4-(1H-tetrazol-1-yl)phenoxy)ethyl)cyclopropyl)piperidin-1-yl)pyrimidine;
5-ethyl-2-(4-((1S,2S)-2-(2-(3-fluoro-4-(1H-tetrazol-1-yl)phenoxy)ethyl)cyclopropyl)piperidin-1-yl)pyrimidine,
5-ethyl-2-(4-((1S,2S)-2-(2-(2-fluoro-4-(1H-tetrazol-1-yl)phenoxy)ethyl)cyclopropyl)piperidin-1-yl)pyrimidine;
5-chloro-2-(4-((1S,2S)-2-(2-(2-fluoro-4-(1H-tetrazol-1-yl)phenoxy)ethyl)cyclopropyl)piperidin-1-yl)pyrimidine;
1-(azetidin-1-yl)-2-(4(2-((1S,2S)-2-(1-(5-chloropyrimidin-2-yl)piperidin-4-yl)cyclopropyl)ethoxy)-2-fluorophenyl)ethanone;
1-(4-(2-((1R,2R)-2-(1-(5-ethylpyrimidin-2-yl)piperidin-4-yl)cyclopropyl)ethoxy)-2-fluorophenyl)imidazolidin-2-one;
1-(azetidin-1-yl)-2-(4-(2-((1R,2R)-2-(1-(5-ethylpyrimidin-2-yl)piperidin-4-yl)cyclopropyl)ethoxy)-3-fluorophenyl)ethanone;
1-(azetidin-1-yl)-2-(4-(2-((1R,2R)-2-(1-(5-ethylpyrimidin-2-yl)piperidin-4-yl)cyclopropyl)ethoxy)-2-fluorophenyl)ethanone;
2-(4-(2-((1S,2S)-2-(1-(5-ethylpyrimidin-2-yl)piperidin-4-yl)cyclopropyl)ethoxy)-2-fluorophenyl)-N,N-dimethylacetamide,
2-(4-(2-((1R,2R)-2-(1-(5-ethylpyrimidin-2-yl)piperidin-4-yl)cyclopropyl)ethoxy)-2-fluorophenyl)-N,N-dimethylacetamide;
1-(azetidin-1-yl)-2-(2-fluoro-4-(2-((1S,2S)-2-(1-(5-(methoxymethyl)pyrimidin-2-yl)piperidin-4-yl)cyclopropyl)ethoxy)phenyl)ethanone;
2-(4-(2-((1S,2S)-2-(1-(5-ethylpyrimidin-2-yl)piperidin-4-yl)cyclopropyl)ethoxy)-2,6-difluorophenyl)-N,N-dimethylacetamide;
1-(azetidin-1-yl)-2-(4-(2-((1S,2S)-2-(1-(5-ethylpyrimidin-2-yl)piperidin-4-yl)cyclopropyl)ethoxy)-2,6-difluorophenyl)ethanone;
1-(azetidin-1-yl)-2-(2-fluoro-4-(2-((1R,2R)-2-(1-(5-(methoxymethyl)pyrimidin-2-yl)piperidin-4-yl)cyclopropyl)ethoxy)phenyl)ethanone;
2-(2-fluoro-4-(2-((1S,2S)-2-(1-(5-(methoxymethyl)pyrimidin-2-yl)piperidin-4-yl)cyclopropyl)ethoxy)phenyl)-N,N-dimethylacetamide;
2-(2-fluoro-4-(2-((1R,2R)-2-(1-(5-(methoxymethyl)pyrimidin-2-yl)piperidin-4-yl)cyclopropyl)ethoxy)phenyl)-N,N-dimethylacetamide;
1-(azetidin-1-yl)-2-(4-(2-((1R,2R)-2-(1-(5-ethylpyrimidin-2-yl)piperidin-4-yl)cyclopropyl)ethoxy)-2,6-difluorophenyl)ethanone;
2-(4-(2-((1R,2R)-2-(1-(5-ethylpyrimidin-2-yl)piperidin-4-yl)cyclopropyl)ethoxy)-2,6-difluorophenyl)-N,N-dimethylacetamide;
1-(azetidin-1-yl)-2-(2,6-difluoro-4-(2-((1R,2R)-2-(1-(5-(methoxymethyl)pyrimidin-2-yl)piperidin-4-yl)cyclopropyl)ethoxy)phenyl)ethanone;
2-(2,6-difluoro-4-(2-((1R,2R)-2-(1-(5-(methoxymethyl)pyrimidin-2-yl)piperidin-4-yl)cyclopropyl)ethoxy)phenyl)-N,N-dimethylacetamide;
5-chloro-2-(4-((1R,2R)-2-(2-(2-fluoro-4-(1H-tetrazol-1-yl)phenoxy)ethyl)cyclopropyl)piperidin-1-yl)pyrimidine;
5-chloro-2-(4-((1S,2S)-2-(2-(3-fluoro-4-(1H-tetrazol-1-yl)phenoxy)ethyl)cyclopropyl)piperidin-1-yl)pyrimidine;
5-ethyl-2-(4-((1R,2R)-2-(2-(2-fluoro-4-(1H-tetrazol-1-yl)phenoxy)ethyl)cyclopropyl)piperidin-1-yl)pyrimidine;
1-(4-(2-((1R,2R)-2-(1-(5-chloropyrimidin-2-yl)piperidin-4-yl)cyclopropyl)ethoxy)-2-fluorophenyl)imidazolidin-2-one;
1-(azetidin-1-yl)-2-(4-(2-((1S,2S)-2-(1-(5-ethylpyrimidin-2-yl)piperidin-4-yl)cyclopropyl)ethoxy)-3-fluorophenyl)ethanone;
1-(azetidin-1-yl)-2-(3-fluoro-4-(2-((1R,2R)-2-(1-(5-(methoxymethyl)pyrimidin-2-yl)piperidin-4-yl)cyclopropyl)ethoxy)phenyl)ethanone;
2-(4-(2-((1S,2S)-2-(1-(5-chloropyrimidin-2-yl)piperidin-4-yl)cyclopropyl)ethoxy)-2-fluorophenyl)-5-methyl-1,3,4-oxadiazole;
5-(4-((1S,2S)-2-(2-(3-fluoro-4-(methylsulfonyl)phenoxy)ethyl)cyclopropyl)piperidin-1-yl)-3-isopropyl-1,2,4-oxadiazole;
5-(4-((1S,2S)-2-(2-(2-fluoro-4-(methylsulfonyl)phenoxy)ethyl)cyclopropyl)piperidin-1-yl)-3-isopropyl-1,2,4-oxadiazole;
5-(4-((1S,2S)-2-(2-(2-fluoro-4-(1H-tetrazol-1-yl)phenoxy)ethyl)cyclopropyl)piperidin-1-yl)-3-isopropyl-1,2,4-oxadiazole;
5-(4-((1R,2R)-2-(2-(2-fluoro-4-(1H-tetrazol-1-yl)phenoxy)ethyl)cyclopropyl)piperidin-1-yl)-3-isopropyl-1,2,4-oxadiazole;
1-(azetidin-1-yl)-2-(2-fluoro-4-(2-((1S,2S)-2-(1-(3-isopropyl-1,2,4-oxadiazol-5-yl)piperidin-4-yl)cyclopropyl)ethoxy)phenyl)ethanone;
1-(azetidin-1-yl)-2-(3-fluoro-4-(2-((1S,2S)-2-(1-(3-isopropyl-1,2,4-oxadiazol-5-yl)piperidin-4-yl)cyclopropyl)ethoxy)phenyl)ethanone;
5-(4-((1S,2S)-2-(2-(4-(ethylsulfonyl)-2-fluorophenoxy)ethyl)cyclopropyl)piperidin-1-yl)-3-isopropyl-1,2,4-oxadiazole;
1-(azetidin-1-yl)-2-(2-fluoro-4-(2-((1R,2R)-2-(1-(3-isopropyl-1,2,4-oxadiazol-5-yl)piperidin-4-yl)cyclopropyl)ethoxy)phenyl)ethanone;
1-(azetidin-1-yl)-2-(2-fluoro-4-(2-((1R,2R)-2-(1-(3-(methoxymethyl)-1,2,4-oxadiazol-5-yl)piperidin-4-yl)cyclopropyl)ethoxy)phenyl)ethanone;
1-(azetidin-1-yl)-2-(3-fluoro-4-(2-(1R,2R)-2-(1-(3-isopropyl-1,2,4-oxadiazol-5-yl)piperidin-4-yl)cyclopropyl)ethoxy)phenyl)ethanone;
2-(2-fluoro-4-(2-((1R,2R)-2-(1-(3-isopropyl-1,2,4-oxadiazol-5-yl)piperidin-4-yl)cyclopropyl)ethoxy)phenyl)-N,N-dimethylacetamide;
2-(2-fluoro-4-(2-((1S,2S)-2-(1-(3-isopropyl-1,2,4-oxadiazol-5-yl)piperidin-4-yl)cyclopropyl)ethoxy)phenyl)-N,N-dimethylacetamide;
2-(2,6-difluoro-4-(2-((1S,2S)-2-(1-(3-isopropyl-1,2,4-oxadiazol-5-yl)piperidin-4-yl)cyclopropyl)ethoxy)phenyl)-N,N-dimethylacetamide;
2-(2,6-difluoro-4-(2-((1S,2S)-2-(1-(3-(methoxymethyl)-1,2,4-oxadiazol-5-yl)piperidin-4-yl)cyclopropyl)ethoxy)phenyl)-N,N-dimethylacetamide;
1-(azetidin-1-yl)-2-(2,6-difluoro-4-(2-((1R,2R)-2-(1-(3-isopropyl-1,2,4-oxadiazol-5-yl)piperidin-4-yl)cyclopropyl)ethoxy)phenyl)ethanone;
1-(azetidin-1-yl)-2-(2,6-difluoro-4-(2-((1R,2R)-2-(1-(3-(methoxymethyl)-1,2,4-oxadiazol-5-yl)piperidin-4-yl)cyclopropyl)ethoxy)phenyl)ethanone;
2-(2,6-difluoro-4-(2-((1R,2R)-2-(1-(3-isopropyl-1,2,4-oxadiazol-5-yl)piperidin-4-yl)cyclopropyl)ethoxy)phenyl)-N,N-dimethylacetamide;
1-(azetidin-1-yl)-2-(2,6-difluoro-4-(2-((1S,2S)-2-(1-(3-isopropyl-1,2,4-oxadiazol-5-yl)piperidin-4-yl)cyclopropyl)ethoxy)phenyl)ethanone;
1-(azetidin-1-yl)-2-(2,6-difluoro-4-(2-((1S,2S)-2-(1-(3-(methoxymethyl)-1,2,4-oxadiazol-5-yl)piperidin-4-yl)cyclopropyl)ethoxy)phenyl)ethanone;
2-(2,6-difluoro-4-(2-((1R,2R)-2-(1-(3-(methoxymethyl)-1,2,4-oxadiazol-5-yl)piperidin-4-yl)cyclopropyl)ethoxy)phenyl)-N,N-dimethylacetamide;
5-(4-((1R,2R)-2-(2-(2-fluoro-4-(methylsulfonyl)phenoxy)ethyl)cyclopropyl)piperidin-1-yl)-3-isopropyl-1,2,4-oxadiazole;
5-(4-((1R,2R)-2-(2-(3-fluoro-4-(1H-tetrazol-1-yl)phenoxy)ethyl)cyclopropyl)piperidin-1-yl)-3-isopropyl-1,2,4-oxadiazole;
5-(4-((1R,2R)-2-(2-(3-fluoro-4-(methylsulfonyl)phenoxy)ethyl)cyclopropyl)piperidin-1-yl)-3-isopropyl-1,2,4-oxadiazole;
1-(3-fluoro-4-(2-((1S,2S)-2-(1-(3-isopropyl-1,2,4-oxadiazol-5-yl)piperidin-4-yl)cyclopropyl)ethoxy)phenyl)imidazolidin-2-one;
5-(4-((1S,2S)-2-(2-(2-fluoro-4-(1H-tetrazol-1-yl)phenoxy)ethyl)cyclopropyl)piperidin-1-yl)-3-(methoxymethyl)-1,2,4-oxadiazole;
5-(4-((1R,2R)-2-(2-(2-fluoro-4-(1H-tetrazol-1-yl)phenoxy)ethyl)cyclopropyl)piperidin-1-yl)-3-(methoxymethyl)-1,2,4-oxadiazole;
1-(azetidin-1-yl)-2-(2-fluoro-4-(2-((1S,2S)-2-(1-(3-(methoxymethyl)-1,2,4-oxadiazol-5-yl)piperidin-4-yl)cyclopropyl)ethoxy)phenyl)ethanone;
5-(4-((1R,2R)-2-(2-(4-(ethylsulfonyl)-2-fluorophenoxy)ethyl)cyclopropyl)piperidin-1-yl)-3-isopropyl-1,2,4-oxadiazole;
2-(2-fluoro-4-(2-((1R,2R)-2-(1-(3-(methoxymethyl)-1,2,4-oxadiazol-5-yl)piperidin-4-yl)cyclopropyl)ethoxy)phenyl)-N,N-dimethylacetamide;
1-methylcyclopropyl 4-((1S,2S)-2-(2-(3-fluoro-4-(methylsulfonyl)phenoxy)ethyl)cyclopropyl)piperidine-1-carboxylate;
1-methylcyclopropyl 4-((1R,2R)-2-(2-(4-(2-(azetidin-1-yl)-2-oxoethyl)-3-fluorophenoxy)ethyl)cyclopropyl)piperidine-1-carboxylate;
1-methylcyclopropyl 4-((1S,2S)-2-(2-(2-fluoro-4-(1H-tetrazol-1-yl)phenoxy)ethyl)cyclopropyl)piperidine-1-carboxylate;
1-methylcyclopropyl 4-((1S,2S)-2-(2-(4-(2-(azetidin-1-yl)-2-oxoethyl)-3-fluorophenoxy)ethyl)cyclopropyl)piperidine-1-carboxylate;
1-methylcyclopropyl 4-((1R,2R)-2-(2-(4-(2-(azetidin-1-yl)-2-oxoethyl)-2-fluorophenoxy)ethyl)cyclopropyl)piperidine-1-carboxylate;
1-methylcyclopropyl 4-((1S,2S)-2-(2-(4-(2-(dimethylamino)-2-oxoethyl)-3-fluorophenoxy)ethyl)cyclopropyl)piperidine-1-carboxylate;
1-methylcyclopropyl 4-((1R,2R)-2-(2-(4-(2-(dimethylamino)-2-oxoethyl)-3-fluorophenoxy)ethyl)cyclopropyl)piperidine-1-carboxylate;
1-methylcyclopropyl 4-((1S,2S)-2-(2-(4-(2-(azetidin-1-yl)-2-oxoethyl)-3,5-difluorophenoxy)ethyl)cyclopropyl)piperidine-1-carboxylate;
1-methylcyclopropyl 4-((1S,2S)-2-(2-(4-(2-(dimethylamino)-2-oxoethyl)-3,5-difluorophenoxy)ethyl)cyclopropyl)piperidine-1-carboxylate;
1-methylcyclopropyl 4-((1R,2R)-2-(2-(4-(2-(azetidin-1-yl)-2-oxoethyl)-3,5-difluorophenoxy)ethyl)cyclopropyl)piperidine-1-carboxylate;
1-methylcyclopropyl 4-((1R,2R)-2-(2-(4-(2-(dimethylamino)-2-oxoethyl)-3,5-difluorophenoxy)ethyl)cyclopropyl)piperidine-1-carboxylate;
1-methylcyclopropyl 4-((1S,2S)-2-(2-(3-fluoro-4-(1H-tetrazol-1-yl)phenoxy)ethyl)cyclopropyl)piperidine-1-carboxylate;
1-methylcyclopropyl 4-((1R,2R)-2-(2-(3-fluoro-4-(1H-tetrazol-1-yl)phenoxy)ethyl)cyclopropyl)piperidine-1-carboxylate;
1-methylcyclopropyl 4-((1R,2R)-2-(2-(2-fluoro-4-(1H-tetrazol-1-yl)phenoxy)ethyl)cyclopropyl)piperidine-1-carboxylate;
1-methylcyclopropyl 4-((1S,2S)-2-(2-(4-(2-(azetidin-1-yl)-2-oxoethyl)-2-fluorophenoxy)ethyl)cyclopropyl)piperidine-1-carboxylate;
5-chloro-2-(4-((1R,2R)-2-(2-(5-(methylsulfonyl)pyridin-2-yloxy)ethyl)cyclopropyl)piperidin-1-yl)pyrimidine;
3-isopropyl-5-(4-((1R,2R)-2-(2-(5-(methylsulfonyl)pyridin-2-yloxy)ethyl)cyclopropyl)piperidin-1-yl)-1,2,4-oxadiazole;
5-(4-((1S,2S)-2-(2-((5-(ethylsulfonyl)pyridin-2-yl)oxy)ethyl)cyclopropyl)piperidin-1-yl)-3-isopropyl-1,2,4-oxadiazole;
3-cyclopropyl-5-(4-((1S,2S)-2-(2-((5-(methylsulfonyl)pyridin-2-yl)oxy)ethyl)cyclopropyl)piperidin-1-yl)-1,2,4-oxadiazole;
5-(4-((1S,2S)-2-(2-((5-(methylsulfonyl)pyridin-2-yl)oxy)ethyl)cyclopropyl)piperidin-1-yl)-3-propyl-1,2,4-oxadiazole;
3-cyclobutyl-5-(4-((1S,2S)-2-(2-((5-(methylsulfonyl)pyridin-2-yl)oxy)ethyl)cyclopropyl)piperidin-1-yl)-1,2,4-oxadiazole;
1-(azetidin-1-yl)-2-(6-(2-((1R,2R)-2-(1-(5-ethylpyrimidin-2-yl)piperidin-4-yl)cyclopropyl)ethoxy)pyridin-3-yl)ethanone;
1-(azetidin-1-yl)-2-(6-(2-((1S,2S)-2-(1-(5-ethylpyrimidin-2-yl)piperidin-4-yl)cyclopropyl)ethoxy)pyridin-3-yl)ethanone;
2-(2-(2-((1R,2R)-2-(1-(5-ethylpyrimidin-2-yl)piperidin-4-yl)cyclopropyl)ethoxy)-4,6-difluorophenyl)-N,N-dimethylacetamide;
1-(azetidin-1-yl)-2-(2-(2-((1S,2S)-2-(1-(5-ethylpyrimidin-2-yl)piperidin-4-yl)cyclopropyl)ethoxy)-4,6-difluorophenyl)ethanone;
1-(azetidin-1-yl)-2-(2-(2-((1R,2R)-2-(1-(5-ethylpyrimidin-2-yl)piperidin-4-yl)cyclopropyl)ethoxy)-4,6-difluorophenyl)ethanone;
1-methylcyclopropyl 4-((1S,2S)-2-(2-(5-(2-(azetidin-1-yl)-2-oxoethyl)pyridin-2-yloxy)ethyl)cyclopropyl)piperidine-1-carboxylate;
1-(azetidin-1-yl)-2-(6-(2-((1S,2S)-2-(1-(3-isopropyl-1,2,4-oxadiazol-5-yl)piperidin-4-yl)cyclopropyl)ethoxy)pyridin-3-yl)ethanone;
2-(2,4-difluoro-6-(2-((1R,2R)-2-(1-(3-isopropyl-1,2,4-oxadiazol-5-yl)piperidin-4-yl)cyclopropyl)ethoxy)phenyl)-N,N-dimethylacetamide;
N-(2-((1R,2S)-2-(1-(5-chloropyrimidin-2-yl)piperidin-4-yl)cyclopropyl)ethyl)-5-(1H-tetrazol-1-yl)pyrimidin-2-amine;
N-(2-((1R,2S)-2-(1-(5-(methoxymethyl)pyrimidin-2-yl)piperidin-4-yl)cyclopropyl)ethyl)-5-(1H-tetrazol-1-yl)pyrimidin-2-amine;
N-(2-((1R,2S)-2-(1-(5-ethylpyrimidin-2-yl)piperidin-4-yl)cyclopropyl)ethyl)-5-(1H-tetrazol-1-yl)pyrimidin-2-amine;
N-(2-((1S,2R)-2-(1-(3-isopropyl-1,2,4-oxadiazol-5-yl)piperidin-4-yl)cyclopropyl)ethyl)-5-(1H-tetrazol-1-yl)pyrimidin-2-amine;
(1S,2R)-2-(1-(5-chloropyrimidin-2-yl)piperidin-4-yl)-1-(2-(4-(methylsulfonyl)phenoxy)ethyl)cyclopropanecarbonitrile; or
N-(2-((1R,2S)-2-(1-(5-chloropyrimidin-2-yl)piperidin-4-yl)cyclopropyl)ethyl)-4-(methylsulfonyl)aniline;
or a pharmaceutically acceptable salt thereof.

15. The compound of claim 1, which is:
5-methyl-2-(4-((1R,2S)-2-(((4-(methylsulfonyl)benzyl)oxy)methyl)cyclopropyl)piperidin-1-yl)pyrimidine;
5-ethyl-2-(4-((1R,2S)-2-(((2-fluoro-4-(methylsulfonyl)benzyl)oxy)methyl)cyclopropyl)piperidin-1-yl)pyrimidine;
5-ethyl-2-(4-((1R,2S)-2-(((4-(methylsulfonyl)benzyl)oxy)methyl)cyclopropyl)piperidin-1-yl)pyrimidine;
1-methylcyclopropyl 4-((1R,2S)-2-(((3-fluoro-4-(methylsulfonyl)benzyl)oxy)methyl)cyclopropyl)piperidine-1-carboxylate;
1-methylcyclopropyl 4-((1R,2S)-2-(((4-(methylsulfonyl)benzyl)oxy)methyl)cyclopropyl)piperidine-1-carboxylate;
3-isopropyl-5-(4-((1R,2S)-2-((4-(methylsulfonyl)benzyloxy)methyl)cyclopropyl)piperidin-1-yl)-1,2,4-oxadiazole;
5-(4-((1R,2S)-2-((3-fluoro-4-(methylsulfonyl)benzyl)oxy)methyl)cyclopropyl)piperidin-1-yl)-3-isopropyl-1,2,4-oxadiazole;
1-(azetidin-1-yl)-2-(2,6-difluoro-4-(2-((1S,2S)-2-(1-(3-isopropyl-1,2,4-oxadiazol-5-yl)piperidin-4-yl)cyclopropyl)ethoxy)phenyl)ethanone;
1-methylcyclopropyl 4-((1R,2R)-2-(2-(4-(2-(azetidin-1-yl)-2-oxoethyl)-3-fluorophenoxy)ethyl)cyclopropyl)piperidine-1-carboxylate;
1-methylcyclopropyl 4-((1R,2R)-2-(2-(4-(2-(azetidin-1-yl)-2-oxoethyl)-3,5-difluorophenoxy)ethyl)cyclopropyl)piperidine-1-carboxylate;
or a pharmaceutically acceptable salt thereof.

16. A pharmaceutical composition comprising a compound of claim 1, or a pharmaceutically acceptable salt thereof, and a pharmaceutically acceptable carrier.

17-18. (canceled)

19. A method for the treatment of diabetes comprising administering to an individual a pharmaceutical composition comprising the compound of claim 1 or a pharmaceutically acceptable salt thereof.

20. (canceled)
